SEQUENCE LISTING (1 sequence)

SEQ ID NO: 1 - DNA, 16 bp, Adeno-associated virus-2
gcgcgctcgctcgctc

(19) United States
(12) Patent Application Publication
(10) Pub. No.: US 2022/0288231 A1
(43) Pub. Date: Sep. 15, 2022

(54) METHODS AND COMPOSITIONS FOR REDUCING GENE OR NUCLEIC ACID THERAPY-RELATED IMMUNE RESPONSES

(71) Applicant: Generation Bio Co., Cambridge, MA (US)

(72) Inventors: Sebastian Aguirre, Cambridge, MA (US); Ching-Hung Chang, Arlington, MA (US); Debra Klatte, Cambridge, MA (US)

(21) Appl. No.: 17/617,336

(22) PCT Filed: Aug. 11, 2020
(86) PCT No.: PCT/US2020/045701

§ 371 Date: Dec. 8, 2021

Related U.S. Application Data
(60) Provisional application No. 62/885,682, filed on Aug. 12, 2019.
(60) Provisional application No. 62/904,935, filed on Sep. 24, 2019.

(51) Int. Cl. A61K 48/00; A61K 31/675; A61K 31/519; A61K 31/501; C12N 15/85

(52) U.S. Cl. CPC A61K 48/005; A61K 31/675; A61K 31/519; A61K 31/501; A61K 48/0008; C12N 15/85

(57) ABSTRACT

This disclosure provides methods and pharmaceutical compositions for attenuating immune response in a subject suffering from a genetic disorder and receiving gene or nucleic acid therapy. The pharmaceutical compositions and formulations may include immunosuppressants, such as protein kinase inhibitors, including tyrosine kinase inhibitors (TKIs), in conjunction with various types of therapeutic nucleic acids (TNAs) and carriers (e.g., lipid nanoparticles).

### RELATED APPLICATIONS

This application claims priority to U.S. Provisional Application No. 62/885,682, filed on Aug. 12, 2019 and U.S. Provisional Application No. 62/904,935, filed on Sep. 24, 2019, the contents of each of which are hereby incorporated by reference in their entireties.

### SEQUENCE LISTING

The instant application contains a Sequence Listing which has been submitted electronically in ASCII format. Said ASCII copy, created on Aug. 10, 2020, is named 131698-07420_SL.txt and is 502 bytes in size.

### TECHNICAL FIELD

The present invention relates to the field of gene and nucleic acid therapy, including the use of a set of antagonists of cytokine signaling and production pathways to reduce proinflammatory immune responses against therapeutic nucleic acid vectors.

### BACKGROUND

Gene therapy aims to improve clinical outcomes for patients suffering from either genetic disorders or acquired diseases caused by an aberrant gene expression profile. Various types of gene therapy that deliver therapeutic nucleic acids into a patient's cells as a drug to treat disease have been developed to date. Generally, gene therapy involves treatment or prevention of medical conditions resulting from defective genes or abnormal regulation or expression, e.g., under- or over-expression, that can result in a disorder, disease, or malignancy. For example, a disease or disorder caused by a defective gene might be treated by delivery of a corrective genetic material to a subject to supplement the defective gene and bolster the wildtype copy of the gene by providing a wild type copy of the gene. In some cases, treatment is achieved by delivery of therapeutic nucleic acid molecules that modulate expression of the defective gene at the transcriptional of translational level, either providing an antisense nucleic acid that binds the target DNA or mRNA that brings down expression levels of the defective gene, or by transferring wildtype mRNA to increase correct copies of the gene.

In particular, human monogenic disorders have been treated by the delivery and expression of a normal gene to the target cells. Delivery and expression of a corrective gene in the patient's target cells can be carried out via numerous methods, including the use of engineered viral gene delivery vectors, and potentially plasmids, minigenes, oligonucleotides, minicircles, or variety of closed-ended DNAs. Among the many virus-derived vectors available (e.g., recombinant retrovirus, recombinant lentivirus, recombinant adenovirus, and the like), recombinant adeno-associated virus (rAAV) is gaining acceptance as a versatile, as well as relatively reliable, vector in gene therapy. However, viral vectors, such as adeno-associated vectors, can be highly immunogenic and elicit humoral and cell-mediated immunity that can compromise efficacy, particularly with respect to re-administration.

Molecular sequences and structural features encoded in the AAV viral genome/vector have evolved to promote episomal stability, viral gene expression and interact with the host's immune system. AAV vectors contain hairpin DNA structures conserved throughout the AAV family, which play critical roles in essential functions of AAV, the ability to tap into the host's genome and replicate themselves, while escaping the surveillance system of the host.

However, some of these gene therapy modalities suffer greatly from the immune related adverse events, which are closely related to host's own defensive mechanism against the therapeutic nucleic acid. For example, the immune system has two general mechanisms for combating infectious diseases that have been implicated in causing adverse events in the recipients of therapy. The first is known as the “innate” immune response that is typically triggered within minutes of infection and serves to limit the pathogen's spread in vivo. The host recognizes conserved determinants expressed by a diverse range of infectious microorganisms, but absent from the host, and these determinants stimulate elements of the host's innate immune system to produce immunomodulatory cytokines and polyreactive IgM antibodies. The second and subsequent mechanism is known as an “adaptive” or antigen specific immune response, which typically generated against determinants expressed uniquely by the pathogen. The innate and adaptive immune responses are mainly activated and modulated by a set of type I interferons (IFNs) through a set of signaling pathways that are activated by specific type of nucleic acids.

IFNs, namely IFNα and IFNβ, are polypeptides produced and secreted by infected cells after the sensing of microbial products by pattern-recognition receptors (PRRs) and by cytokines and have three major functions: (1) they induce cell-intrinsic antimicrobial states in infected and neighboring cells that limit the spread of infectious agents, particularly viral pathogens; (2) they modulate innate immune responses in a balanced manner that promotes antigen presentation and natural killer cell functions while restraining pro-inflammatory pathways and cytokine production; and (3) they activate the adaptive immune system, thus promoting the development of high-affinity antigen-specific T and B cell responses and immunological memory. Thus, type I IFNs are protective in acute viral infections, but can have either protective or deleterious roles in bacterial infections and autoimmune diseases. Most cell types produce IFNβ, whereas hematopoietic cells, particularly plasmacytoid dendritic cells, are the predominant producers of IFNα. Canonical type I IFN signaling activates the Janus kinase (JAK)-signal transducer and activator of transcription (STAT) pathway, leading to transcription of IFN-stimulated genes (ISGs). Host, pathogen and environmental factors regulate the responses of cells to this signaling pathway and, thus, calibrate host defenses while limiting tissue damage and preventing autoimmunity.

Although conceptually elegant, the prospect of using nucleic-acid molecules for gene therapy for treating human diseases remains uncertain. The main cause of this uncertainty is the apparent adverse events relating to host's innate immune response to nucleic acid therapeutics and, thus, the way in which these materials modulate expression of their intended targets in the context of the immune response. The current state of the art surrounding the creation, function, behavior and optimization of nucleic acid molecules that may be adopted for clinical applications has a particular focus on: (1) antisense oligonucleotides and duplex RNAs that directly regulate translation and gene expression; (2) transcriptional gene silencing RNAs that result in long-term epigenetic modifications; (3) antisense oligonucleotides that interact with and alter gene splicing patterns; (4) creation of synthetic or viral vectors that mimic physiological functionalities of naturally occurring AAV or lentiviral genome; and (5) the in vivo delivery of therapeutic oligonucleotides. However, despite the advances made in the development of nucleic acid therapeutics that are evident in recent clinical achievements, the field of gene therapy is still severely limited by unwanted adverse events in recipients triggered by the therapeutic nucleic acids, themselves.

Accordingly, there is a strong need in the field for a new technology that effectively reduces, ameliorates, mitigates, prevents or maintains the immune response systems that are triggered by nucleic acid therapeutics.

### SUMMARY

This disclosure provides methods and pharmaceutical compositions for attenuating immune response in a subject suffering from a genetic disorder and receiving gene or nucleic acid therapy (“nucleic acid therapeutics” or“therapeutic nucleic acid” (TNA)). The pharmaceutical compositions and formulations may include immunosuppressants, such as protein kinase inhibitors (PKIs), including tyrosine kinase inhibitors (TKIs), in conjunction with various types of therapeutic nucleic acids (TNA) and carriers (e.g., lipid nanoparticle). In some embodiments, a pharmaceutical formulation can be prepared by combining a TNA with a PKI according to the instruction prior to administration.

The present invention also contemplates a kit for combining the TNA and the PKI to make a pharmaceutical formulation to be administered to a subject.

The methods generally include use of protein kinase inhibitors (e.g., PKIs) for preventing, reducing, attenuating or even eliminating immune responses associated with administration of a therapeutic nucleic acids.

In one aspect, disclosed herein is a pharmaceutical composition comprising a therapeutic nucleic acid and a tyrosine kinase inhibitor (TKI).

In one embodiment, the therapeutic nucleic acid is an RNA molecule, or a derivative thereof. In one embodiment, the RNA molecule is an antisense oligonucleotide. In one embodiment, the antisense oligonucleotide is an antisense RNA. In one embodiment, the RNA is RNA interference (RNAi).

In one embodiment, the therapeutic nucleic acid is an mRNA molecule.

In one embodiment, the therapeutic nucleic acid is a DNA molecule, or a derivative thereof.

In one embodiment, the therapeutic nucleic acid is a DNA antisense oligonucleotide. In one embodiment, the DNA antisense oligonucleotide is morpholino based nucleic acid. In one embodiment, the morpholino based nucleic acid is a phosphorodiamidate morpholino oligomer (PMO).

In one embodiment, the therapeutic nucleic acid is a closed-ended DNA (ceDNA). In one embodiment, the ceDNA comprises an expression cassette comprising a promoter sequence and a transgene. In one embodiment, the ceDNA comprises expression cassette comprising a polyadenylation sequence. In one embodiment, the ceDNA comprises at least one inverted terminal repeat (ITR) flanking either 5′ or 3′ end of the expression cassette. In one embodiment, the expression cassette is flanked by two ITRs, wherein the two ITRs comprise one 5′ ITR and one 3′ ITR. In one embodiment, the expression cassette is connected to an ITR at 3′ end (3′ ITR). In one embodiment, the expression cassette is connected to an ITR at 5′ end (5′ ITR). In one embodiment, the ceDNA further comprises a spacer sequence between a 5′ ITR and the expression cassette.

In one embodiment, the ceDNA further comprises a spacer sequence between a 3′ ITR and the expression cassette. In one embodiment, the spacer sequence is at least 5 base pair long in length. In one embodiment, the spacer sequence is 5 to 200 base pairs long in length. In one embodiment, the spacer sequence is 5 to 500 base pairs long in length.

In one embodiment, the ITR is an ITR derived from an AAV serotype. In one embodiment, the AAV is selected from the group consisting of AAV1, AAV2, AAV3, AAV4, AAV5, AAV6, AAV7, AAV8, AAV9, AAV10, AAV11 and AAV12. In one embodiment, the ITR is derived from an ITR of goose virus. In one embodiment, the ITR is derived from a B19 virus ITR. In one embodiment, the ITR is a wild-type ITR from a parvovirus. In one embodiment, the ITR is a mutant ITR. In one embodiment, the ceDNA comprises two mutant ITRs in both 5′ and 3′ ends of the expression cassette.

In one embodiment, the ceDNA has a nick or a gap.

In one embodiment, the ceDNA is synthetically produced in a cell-free environment.

In one embodiment, the ceDNA is produced in a cell. In one embodiment, the ceDNA is produced in insect cells. In one embodiment, the insect cell is Sf9. In one embodiment, the ceDNA is produced in a mammalian cell. In one embodiment, the mammalian cell is human cell line.

In one embodiment, the therapeutic nucleic acid is a closed-ended DNA comprising at least one protelomerase target sequence in its 5′ and 3′ ends of the expression cassette.

In one embodiment, the therapeutic nucleic acid is a dumbbell shaped linear duplex closed-ended DNA comprising two hairpin structures of ITRs in 5′ and 3′ ends of an expression cassette. In one embodiment, the therapeutic nucleic acid is a DNA-based minicircle or a MIDGE. In one embodiment, the therapeutic nucleic acid is a linear covalently closed-ended DNA vector. In one embodiment, the linear covalently closed-ended DNA vector is a ministring DNA. In one embodiment, the therapeutic nucleic acid is a doggybone (dbDNA™) DNA. In one embodiment, the therapeutic nucleic acid is a minigene. In one embodiment, the therapeutic nucleic acid is a plasmid. In one embodiment, the tyrosine kinase inhibitor is a pharmaceutically acceptable salt of the tyrosine kinase inhibitor.

In one embodiment, the composition further comprises an excipient or carrier.

In one embodiment, the pharmaceutical composition comprises a lipid nanoparticle (LNP). In one embodiment, the LNP comprises a cationic lipid. In one embodiment, the LNP comprises polyethylene glyclol (PEG). In one embodiment, the LNP comprises a cholesterol. In one embodiment, the TKI is selected from the group consisting of acalabrutinib, alectinib, baricitinib, afatinib, brigatinib, crizotinib, dacomitinib, dasatinib, lorlatinib, osimertinib, fostamatinib, saracatinib, AG-1478, cobimetinib, ceritinib, lapatinib, gefitinib, erlotinib, ruxolitinib, cerdulatinib, tofacitinib, BMS-986165, vandetinib, and bosutinib. In one embodiment, the TKI is selected from the group consisting of baricitinib, afatinib, brigatinib, dacomitinib, dasatinib, osimertinib, fostamatinib, saracatinib, cobimetinib, ceritinib, ruxolitinib, cerdulatinib, BMS-986165, and tofacitinib. In one embodiment, the TKI is selected from the group consisting of sunitinib, imatinib, sorafenib, dasatinib, entoplestinib, fostamatinib, TAK-659, ruxolitinib, baricitinib, BMS-986165, and tofacitinib.

In one embodiment, the TKI is an inhibitor of STAT1. In one embodiment, the TKI is an inhibitor of STAT2. In one embodiment, the TKI is an inhibitor of spleen tyrosine kinase (Syk). In one embodiment, the Syk inhibitor is fostamatinib. In one embodiment, the Syk inhibitor is cerdulatinib.

In one embodiment, the TKI is an inhibitor of epidermal growth factor receptor (EGFR, aka ErbB-1 or HER-1). In one embodiment, the EGFR inhibitor is afatinib. In one embodiment, the EGFR inhibitor is dacomitinib.

In one embodiment, the TKI is an inhibitor of anaplastic lymphoma kinase (ALK). In one embodiment, the ALK inhibitor is brigatinib. In one embodiment, the ALK inhibitor is alectinib. In one embodiment, the ALK inhibitor is ceritinib. In one embodiment, the ALK inhibitor is lorlatinib. In one embodiment, the ALK inhibitor is crizotinib.

In one embodiment, the TKI is an antagonist of IFN production pathway. In one embodiment, the TKI is an antagonist of IFN signaling pathway. In one embodiment, the TKI is an inhibitor of Janus kinase 1 (Jak1). In one embodiment, the Jak1 inhibitor is ruxolitinib. In one embodiment, the Jak1 inhibitor is baricitinib. In one embodiment, the TKI is an inhibitor of Janus kinase 2 (Jak2). In one embodiment, the Jak2 inhibitor is ruxolitinib. In one embodiment, the Jak2 inhibitor is baricitinib. In one embodiment, the TKI is a Jak1/2 inhibitor selected from the group consisting of ruxolitinib, baricitinib, and tofacitinib.

In one embodiment, the TKI is an inhibitor of tyrosine kinase 2 (Tyk2). In one embodiment, the Tyk2 inhibitor is ruxolitinib. In one embodiment, the Tyk2 inhibitor is tofacitinib. In one embodiment, the Tyk2 inhibitor is BMS-986165.

In one embodiment, the TKI is Abl/Src inhibitor. In one embodiment, the Abl/Src inhibitor is bosutinib.

In another aspect, disclosed herein is a method of treating a genetic disorder in a subject, the method comprising administering to the subject an effective amount of a pharmaceutical composition disclosed herein. In one embodiment, the subject is a human. In one embodiment, the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson's disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), omithine transcarbamylase (OTC) deficiency, Usher syndrome, alpha-1 antitrypsin deficiency, progressive familial intrahepatic cholestasis (PFIC), and Cathepsin A deficiency.

In one embodiment, the genetic disorder is Leber congenital amaurosis (LCA). In one embodiment, the LCA is LCA10. In one embodiment, the genetic disorder is Niemann-Pick disease. In one embodiment, the genetic disorder is Stargardt macular dystrophy. In one embodiment, the genetic disorder is glucose-6-phasphatase (G6Pase) deficiency (glycogen storage disease type I) or Pompe disease (glycogen storage disease type II). In one embodiment, the genetic disorder is hemophilia A (Factor VIII deficiency). In one embodiment, the genetic disorder is hemophilia B (Factor IX deficiency). In one embodiment, the genetic disorder is hunter syndrome (Mucopolysaccharidosis II). In one embodiment, the genetic disorder is cystic fibrosis (CFTR). In one embodiment, the genetic disorder is dystrophic epidermolysis bullosa (DEB). In one embodiment, the genetic disorder is phenylketonuria (PKU). In one embodiment, the genetic disorder is hyaluronidase deficiency.

In another aspect, disclosed herein is a method of treating a genetic disorder in a subject, the method comprising administering to the subject an effective amount of a protein kinase inhibitor (PKI) and an effective amount of a therapeutic nucleic acid (TNA).

In one embodiment, the TNA is an RNA molecule, or a derivative thereof. In one embodiment, the RNA molecule is an antisense oligonucleotide. In one embodiment, the antisense oligonucleotide is an antisense RNA.

In one embodiment, the TNA is an RNA interference molecule. In one embodiment, the TNA is an mRNA molecule. In one embodiment, the TNA is a DNA molecule, or a derivative thereof. In one embodiment, the TNA is peptide nucleic acid (PNA), locked nucleic acid (LNA), or morpholino based antisense oligomer. In one embodiment, the TNA is a DNA antisense oligonucleotide. In one embodiment, the DNA antisense oligonucleotide is morpholino based nucleic acid. In one embodiment, the morpholino based nucleic acid is a phosphorodiamidate morpholino oligomer (PMO).

In one embodiment, the DNA is a closed-ended DNA (ceDNA). In one embodiment, the ceDNA comprises an expression cassette comprising a promoter sequence operatively linked to a transgene. In one embodiment, the ceDNA comprises expression cassette comprising a polyadenylation sequence. In one embodiment, the ceDNA comprises at least one inverted terminal repeat (ITR) flanking either 5′ or 3′ end of the expression cassette. In one embodiment, one ITR is connected to 5′ end (5′ ITR) and another ITR is connected to 3′ ends of the expression cassette. In one embodiment, the ceDNA further comprises a spacer sequence between the 5′ ITR and the expression cassette. In one embodiment, the ceDNA further comprises a spacer sequence between 3′ ITR and the expression cassette. In one embodiment, the spacer sequence is at least 5 base pairs long in length. In one embodiment, the spacer sequence is 5 to 200 base pairs long in length. In one embodiment, the spacer sequence is 5 to 500 base pairs long in length. In one embodiment, the ceDNA has a gap in one strand.

In one embodiment, the ITR is an ITR derived from an AAV serotype. In one embodiment, the AAV serotype is selected from the group consisting of AAV1, AAV2, AAV3, AAV4, AAV5, AAV6, AAV7, AAV8, AAV9, AAV10, AAV11, and AAV12. In one embodiment, the ITR is derived from an ITR of goose virus. In one embodiment, the ITR is derived from a B19 virus ITR. In one embodiment, the ITR is a wild-type ITR derived from a parvovirus. In one embodiment, the ITR is a mutant ITR. In one embodiment, the ceDNA comprises two mutant ITRs flanking both 5′ and 3′ ends of the expression cassette.

In one embodiment, the ceDNA is synthetically produced in a cell-free environment. In one embodiment, the ceDNA is produced in a cell. In one embodiment, the is an insect cell. In one embodiment, the insect cell is Sf9.

In one embodiment, the TNA is a DNA-based minicircle or a MIDGE. In one embodiment, the TNA is closed-ended DNA having at least one protelomerase target sequence. In one embodiment, the TNA is a dumbbell shaped DNA. In one embodiment, the TNA is a doggybone (dbDNA™) DNA. In one embodiment, the TNA is a minigene. In one embodiment, the TNA is a linear covalently closed DNA ministring. In one embodiment, the TNA is a plasmid or bacmid.

In one embodiment, the protein kinase inhibitor is a tyrosine kinase inhibitor (TKI), or a pharmaceutically acceptable salt thereof.

In one embodiment, the TNA is formulated in a pharmaceutically acceptable excipient or carrier. In one embodiment, the carrier comprises a lipid nanoparticle (LNP). In one embodiment, the LNP comprises a cationic lipid. In one embodiment, the LNP comprises PEG. In one embodiment, the LNP comprises a cholesterol. In one embodiment, the LNP comprises the protein kinase inhibitor.

In one embodiment, the protein kinase inhibitor is a tyrosine kinase inhibitor (TKI). In one embodiment, the TKI is selected from the group consisting of acalabrutinib, alectinib, baricitinib, afatinib, brigatinib, crizotinib, dacomitinib, dasatinib, lorlatinib, osimertinib, fostamatinib, saracatinib, AG-1478, cobimetinib, ceritinib, lapatinib, gefitinib, erlotinib, TAK-659, ruxolitinib, cerdulatinib, tofacitinib, vandetinib, and bosutinib. In one embodiment, the TKI is selected from the group consisting of baricitinib, afatinib, brigatinib, dacomitinib, dasatinib, osimertinib, fostamatinib, saracatinib, cobimetinib, ceritinib, ruxolitinib, cerdulatinib, and tofacitinib. In one embodiment, the TKI is selected from the group consisting of sunitinib, imatinib, sorafenib, dasatinib, entoplestinib, fostamatinib, TAK-659, ruxolitinib, baricitinib, and tofacitinib.

In one embodiment, the TKI is an STAT1 inhibitor. In one embodiment, the TKI is an inhibitor of spleen tyrosine kinase (Syk). In one embodiment, the Syk inhibitor is fostamatinib. In one embodiment, the Syk inhibitor is cerdulatinib.

In one embodiment, the TKI is an inhibitor of epidermal growth factor receptor (EGFR, aka ErbB-1 or HER-1). In one embodiment, the EGFR inhibitor is afatinib. In one embodiment, the EGFR inhibitor is dacomitinib.

In one embodiment, the TKI is an inhibitor of anaplastic lymphoma kinase (ALK). In one embodiment, the ALK inhibitor is brigatinib. In one embodiment, the ALK inhibitor is alectinib. In one embodiment, the ALK inhibitor is ceritinib. In one embodiment, the ALK inhibitor is lorlatinib. In one embodiment, the ALK inhibitor is crizotinib.

In one embodiment, the TKI is an antagonist of IFN production pathway. In one embodiment, the TKI is an antagonist of IFN signaling pathway.

In one embodiment, the TKI is an inhibitor of tyrosine kinase 2 (Tyk2). In one embodiment, the TKI is an inhibitor of Janus kinase 1 (Jak1). In one embodiment, the TKI is an inhibitor of Janus kinase 2 (Jak2). In one embodiment, the Tyk2 inhibitor is ruxolitinib. In one embodiment, the Jak1 inhibitor is ruxolitinib. In one embodiment, the Jak2 inhibitor is ruxolitinib. In one embodiment, the Jak1 inhibitor is baricitinib. In one embodiment, the Jak2 inhibitor is baricitinib. In one embodiment, the Tyk2 inhibitor is tofacitinib.

In one embodiment, the TKI is an inhibitor of STAT1 or STAT2. In one embodiment, the TKI is ABL/Src inhibitor.

In one embodiment, the subject is a human from the genetic disorder. In one embodiment, the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, phenylketonuria (PKU), Wilson's disease, congenital hepatic porphyria, inherited disorders of hepatic metabolism, Lesch Nyhan syndrome, sickle cell anemia, thalassemia's, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis (LCA), Stargardt macular dystrophy (ABCA4 deficiency), ornithine transcarbamylase (OTC) deficiency, Usher syndrome, alpha-1 antitrypsin deficiency, progressive familial intrahepatic cholestasis (PFIC), and Cathepsin A deficiency.

In one embodiment, the genetic disorder is Parkinson's disease. In one embodiment, the genetic disorder is Alzheimer's disease. In one embodiment, the genetic disorder is thalassemia. In one embodiment, the genetic disorder is Leber congenital amaurosis (LCA). In one embodiment, the LCA is LCA10. In one embodiment, the genetic disorder is Niemann-Pick disease. In one embodiment, the genetic disorder is Stargardt macular dystrophy. In one embodiment, the genetic disorder is Pompe disease (glycogen storage disease type II). In one embodiment, the genetic disorder is hemophilia A (Factor VIII deficiency). In one embodiment, the genetic disorder is hemophilia B (Factor IX deficiency). In one embodiment, the genetic disorder is hunter syndrome (Mucopolysaccharidosis II). In one embodiment, the genetic disorder is cystic fibrosis (CFTR). In one embodiment, the genetic disorder is dystrophic epidermolysis bullosa (DEB). In one embodiment, the genetic disorder is phenylketonuria (PKU). In one embodiment, the genetic disorder is hyaluronidase deficiency.

In one embodiment, the TKI is administered prior to the administration of the TNA. In one embodiment, the TKI is administered at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least about 2 days, at least about 3 days, at least about 4 days, at least about 5 days, at least about 6 days, at least about 1 week, at least about 2 weeks, at least about 3 weeks, or at least about 4 weeks prior to the administration of the TNA. In one embodiment, the TKI is administered simultaneously as the administration of the TNA.

In one embodiment, the TKI and the TNA are in a liquid solution. In one embodiment, the TKI and TNA are any of the pharmaceutical compositions according to claims 1 to 82.

In one embodiment, the TKI is administered after the administration of the TNA. In one embodiment, the TKI is administered 30 minutes after the administration of the TNA. In one embodiment, the TKI is administered about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or 24 hours after the administration of the therapeutic nucleic acid. In one embodiment, the TKI is administered about 1 day, about 2 days, about 3 days, about 4 days, about 6 days, or about 7 days after the administration of the TNA. In one embodiment, the TKI is administered about 5 hours after the administration of the TNA. In one embodiment, the TKI is administered about 12 hours after the administration of the TNA. In one embodiment, the TKI is administered about 24 hours after the administration of TNA.

In one embodiment, the TKI is administered multiple times, before, concurrently with, and/or after the administration of the TNA. In one embodiment, the TKI is administered multiple times, before and/or after the administration of the TNA, at least within about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or 24 hours.

In one embodiment, the TKI is administered multiple times, before, at the same time, and/or after the administration of the TNA. In one embodiment, the protein kinase inhibitor and TNA are administered by oral, topical, intradermal, intrathecal, intravenous, subcutaneous, intramuscular, intratumoral, intra-articular, intraspinal, spinal, nasal, epidural, rectal, vaginal, transdermal, or transmucosal route.

In one embodiment, the protein kinase inhibitor is administered at a dosage of about 0.5 mg/kg to about 700 mg/kg. In one embodiment, the protein kinase inhibitor is administered at a dosage of about 5 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 150 mg/kg, about 200 mg/kg, about 250 mg/kg, about 300 mg/kg, about 350 mg/kg, about 400 mg/kg, about 450 mg/kg, or about 500 mg/kg. In one embodiment, the protein kinase inhibitor is administered at a dosage of about 200 mg/kg. In one embodiment, the protein kinase inhibitor is administered at a dosage of about 300 mg/kg. In one embodiment, the protein kinase inhibitor is administered at a dosage of about 400 mg/kg. In one embodiment, the protein kinase inhibitor is administered at a dosage of about 500 mg/kg. In one embodiment, the protein kinase inhibitor is administered at a dosage of about 600 mg/kg. In one embodiment, the protein kinase inhibitor is a tyrosine kinase inhibitor.

### BRIEF DESCRIPTION OF THE DRAWINGS

FIG. 1 depicts a schematic representation of an in vitro evaluation strategy for the effect of various immunomodulators (e.g., protein kinase inhibitors) on ceDNA-induced immune responses.

FIG. 2 depicts THP1-ISG cell viability assay results as measured by ATP activity and LDH release at 24 hours after the treatment with selected protein kinases.

FIG. 3 depicts IFN reporter assay results in THP1-ISG cells at 48 hours after ceDNA-LNP treatment as measured by luciferase activity.

FIG. 4 depicts IFN reporter assay results in THP1-ISG cells at 24 after cGAMPs-LNP treatment as measured by luciferase activity.

FIG. 5 depicts THP1-ISG cell viability assay results as measured by ATP activity at 24 hours after the treatment with selected tyrosine kinase inhibitors.

FIG. 6 depicts IFN reporter assay results in THP1-ISG cells at 24 hours after the ceDNA-LNP treatment.

FIG. 7 depicts IFN reporter assay results in a murine cell line (RAW-ISG) using a potent TKI, e.g., TYK2-IN-4, ruxolitinib, and fostamatinib at 0.1 μM, 1.0 μM, and 10 μM concentrations.

FIG. 8 depicts charts demonstrating body weight changes and expression level of a ceDNA reporter construct (Construct A; 5′ ITR-spacer-hAAT (CpG minimal)-luciferase (CpG minimal)-HBB 3′UTR-SV40 polyA-spacer-3′ ITR) in mice treated with a high or low dose of protein kinase inhibitors and 0.2 mg/kg ceDNA.

FIGS. 9A and 9B depict serum levels of proinflammatory cytokines in mice treated with selected protein kinases (antagonists) at a high (H) concentration or low (L) concentration, the cytokines including interferon-α (IFN-α), interferon-γ (IFN-γ), tumor necrosis factor-α (TNF-α), interleukin-6 (IL-6), interleukin-18 (IL-18), and IP-10; polyC: negative control; Construct A: positive control agonist; vehicle: lipid particle (LNPs).

FIG. 10 depicts serum levels of chemokines including MCP-1, MIP-1α (CCL3), MIP-1β (CCL4), and Rantes (CCL5) in mice treated with selected protein kinases at a high (H) concentration or low (L) concentration and ceDNA (Construct A).

FIGS. 11A and 11B depict serum levels of proinflammatory cytokines in mice treated with fostamatinib and ruxolitinib at a high (H) (500 mg/kg and 300 mg/kg, respectively) or low (L) concentration (100 mg/kg and 100 mg/kg, respectively), the cytokines including interferon-α (IFN-α), interferon-γ (IFN-γ), tumor necrosis factor-α (TNF-α), interleukin-6 (IL-6), interleukin-18 (IL-18), and IP-10; polyC: negative control; Construct A: positive control agonist; vehicle: lipid particle (LNPs).

FIG. 12 depicts serum levels of IFN-α and IFN-γ in mice treated with fostamatinib and ruxolitinib at a high (H) (500 mg/kg and 300 mg/kg, respectively) or low (L) concentration (100 mg/kg and 60 mg/kg, respectively) and their body weight changes up to 3 days post injection of ceDNA.

FIGS. 13A and 13B depict dose escalation study results using ceDNA concentrations from 0.2 mg/kg, 0.5, mg/kg, 1 mg/kg, and 2 mg/kg along with ruxolitinib concentration at 300 mg/kg or fostamatinib concentration at 500 mg/kg. Serum levels of IFN-α and IL6, as well as expression levels of luciferase at day 7 are depicted.

FIG. 14 depicts in vivo study results from two different dosing regimen in which 300 mg/kg of ruxolitinib was dosed at either −48 h, −24 h, −1.5 h and 24 h or −0.5 h and 5 h timeline regimen at two different concentrations of ceDNA (0.2 mg/kg or 1.0 mg/kg).

FIG. 15 depicts expression levels of factor IX in mice injected with an LNP formulated FIX ceDNA construct at day 0 and treated with 300 mg/kg of ruxolitinib at −48 h, −24 h, −1.5 h and 24 h timepoints.

FIG. 16 depicts the effect of ruxolitinib on expression of ceDNA in a re-dose regimen model in which ceDNA was re-dosed 35 days after the initial treatment. Ruxolitinib was dosed at −48 h, −24 h, −1.5 h and 24 h at day 0 of ceDNA-luciferase (0.2 mg/kg) treatment (initial dose) and −48 h, −24 h, −1.5 h and 24 h at day 35 of ceDNA (2.0 mg/kg) re-dose. Vehicle: LNP; PolyC: negative control.

FIG. 17 depicts plasma levels of factor IX in mice injected with an LNP formulated FIX ceDNA construct (2.0 mg/kg) at days 0 and 36 and orally dosed with 300 mg/kg of ruxolitinib at day −2, day −1, day 0, day 1 and day 36.

### DETAILED DESCRIPTION

Nucleic acid transfer vectors and therapeutic agents are promising therapeutics for a variety of applications, such as gene expression and modulation thereof. Viral transfer vectors may comprise transgenes that encode proteins or nucleic acids. Examples of such include AAV vectors, microRNA (miRNA), small interfering RNA (siRNA), as well as antisense oligonucleotides that bind mutation sites in messenger RNA (such as small nuclear RNA (snRNA)). Unfortunately, the promise of these therapeutics has not yet been realized, in large part due to cellular and humoral immune responses directed against the viral transfer vector. These immune responses include antibody, B cell and T cell responses, and are often specific to viral antigens of the viral transfer vector, such as viral capsid or coat proteins or peptides thereof.

Currently, many potential patients harbor some level of pre-existing immunity against the viruses on which viral transfer vectors are based. In fact, antibodies against viral nucleic acids (both DNA and RNA) or protein are highly prevalent in the human population. In addition, even if the level of pre-existing immunity is low, for example, due to the low immunogenicity of the viral transfer vector, such low levels may still prevent successful transduction (e.g., Jeune, et al., Human Gene Therapy Methods, 24:59-67 (2013)). Thus, even low levels of pre-existing immunity may hinder the use of a specific viral transfer vector in a patient, and may require a clinician to choose a viral transfer vector based on a virus of a different serotype that may not be as efficacious, or even opt out for a different type of therapy altogether if another viral transfer vector therapy is not available.

Additionally, viral vectors, such as adeno-associated vectors, can be highly immunogenic and elicit humoral and cell-mediated immunity that can compromise efficacy, particularly with respect to re-administration. In fact, cellular and humoral immune responses against a viral transfer vector can develop after a single administration of the viral transfer vector. After viral transfer vector administration, neutralizing antibody titers can increase and remain high for several years, and can reduce the effectiveness of re-administration of the viral transfer vector. Indeed, repeated administration of a viral transfer vector generally results in enhanced, undesired immune responses. In addition, viral transfer vector-specific CD8+ T cells may arise and eliminate transduced cells expressing a desired transgene product on re-exposure to a viral antigen. For example, it has been shown that AAV nucleic acids or capsid antigens can trigger immune-mediated destruction of hepatocytes transduced with an AAV viral transfer vector. For many therapeutic applications, it is thought that multiple rounds of administration of viral transfer vectors are needed for long-term benefits. The ability to do so, however, would be severely limited if re-administration is required in patients.

The problems associated with the use of viral transfer vectors for therapy is further compounded because viral transfer vector antigens can persist for a long period of time. In order for therapy to be successful, it is important to evade or attenuate immune responses against viral transfer vectors. Prior to this invention, however, there was no effective means for which attenuation of a long-term immune response could be achieved.

The inventors have unexpectedly discovered that the problems and limitations noted above can be overcome by practicing the inventions disclosed herein. Methods and compositions are provided to offer solutions to the aforementioned obstacles to effective use of variety of nucleic acid therapeutics, including viral or non-viral (synthetic) transfer vectors, and other nucleic acid therapeutics for treatment. It has been unexpectedly discovered that cellular and in vivo immune responses to DNA transfer vector can be attenuated with the methods and related compositions provided herein. Hence, the methods and compositions can potentially increase the efficacy of treatment with viral transfer vectors and other therapeutic nucleic acid molecules and provide for long-term therapeutic benefits, even if the administration of the viral transfer vector or other nucleic acid therapeutics is repeated.

### I. Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6th Edition, published by Lippincott Williams & Wilkins, Philadelphia, Pa., USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al. Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms, “administration,” “administering” and variants thereof refers to introducing a composition or agent (e.g., a therapeutic nucleic acid or an immunosuppressant as described herein) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone (dbDNA™) DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone (dbDNA™) DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

“Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

An “inhibitory polynucleotide” as used herein refers to a DNA or RNA molecule that reduces or prevents expression (transcription or translation) of a second (target) polynucleotide. Inhibitory polynucleotides include antisense polynucleotides, ribozymes, and external guide sequences. The term “inhibitory polynucleotide” further includes DNA and RNA molecules, e.g., RNAi that encode the actual inhibitory species, such as DNA molecules that encode ribozymes.

As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “interfering RNA” or “RNAi” or “interfering RNA sequence” includes single-stranded RNA (e.g., mature miRNA, ssRNAi oligonucleotides, ssDNAi oligonucleotides), double-stranded RNA (i.e., duplex RNA such as siRNA, Dicer-substrate dsRNA, shRNA, aiRNA, or pre-miRNA), a DNA-RNA hybrid (see, e.g., PCT Publication No. WO 2004/078941), or a DNA-DNA hybrid (see, e.g., PCT Publication No. WO 2004/104199) that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. Interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof. Preferably, the interfering RNA molecules are chemically synthesized. The disclosures of each of the above patent documents are herein incorporated by reference in their entirety for all purposes. The term “RNAi” can include both gene silencing RNAi molecules, and also RNAi effector molecules which activate the expression of a gene. In some embodiments RNAi agents which serve to inhibit or gene silence are useful in the methods, kits and compositions disclosed herein, e.g., to inhibit the immune response.

Interfering RNA includes “small-interfering RNA” or “siRNA,” e.g., interfering RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 18-22, 19-20, or 19-21 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule. As used herein, the term “siRNA” includes RNA-RNA duplexes as well as DNA-RNA hybrids (see, e.g., PCT Publication No. WO 2004/078941).

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to an uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “gene delivery” means a process by which foreign DNA is transferred to host cells for applications of gene therapy.

As used herein, the term “immunosuppressant” refers to a group of small molecules, monoclonal antibodies or polypeptide antagonists that inhibits protein kinases, such as tyrosine kinases.

As used herein, the term “tyrosine kinase inhibitor” or “TKI” refers to a molecule that inhibits tyrosine kinase activity. A tyrosine kinase inhibitor may be, for example, a small molecule inhibitor, a biologic (such as a monoclonal antibody), or a large polypeptide molecule that inhibits the activity of, for example, IFN signaling and production pathways; or any other form of antagonist that can decrease expression or activity of a tyrosine kinase.

As used herein, the phrase “anti-therapeutic nucleic acid immune response”, “anti-transfer vector immune response”, “immune response against a therapeutic nucleic acid”, “immune response against a transfer vector”, or the like refers to any undesired immune response against a therapeutic nucleic acid, viral or non-viral in its origin. In some embodiments, the undesired immune response is an antigen-specific immune response against the viral transfer vector itself. In some embodiments, the immune response is specific to the transfer vector which can be double stranded DNA, single stranded RNA, or double stranded RNA. In other embodiments, the immune response is specific to a sequence of the transfer vector. In other embodiments, the immune response is specific to the CpG content of the transfer vector.

As used herein, an “effective amount” or “therapeutically effective amount” of an active agent or therapeutic agent, such as an immunosuppressant and/or therapeutic nucleic acid, is an amount sufficient to produce the desired effect, e.g., a normalization or reduction of immune responses and expression or inhibition of expression of a target sequence in comparison to the expression level detected in the absence of a therapeutic nucleic acid and/or immunosuppressant. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “increase,” “enhance,” “raise” (and like terms) generally refers to the act of increasing, either directly or indirectly, a concentration, level, function, activity, or behavior relative to the natural, expected, or average, or relative to a control condition.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition. By “decrease,” “decreasing,” “reduce,” or “reducing” of an immune response by an immunosuppressant is intended to mean a detectable decrease of an immune response to a given immunosuppressant. The amount of decrease of an immune response by the immunosuppressant may be determined relative to the level of an immune response in the presence of an immunosuppressant. A detectable decrease can be about 5%, 10%, 15%, 20%, 25%, 30%, 35%, 40%, 45%, 50%, 55%, 60%, 65%, 70%, 75%, 80%, 85%, 90%, 95%, 100%, or more lower than the immune response detected in the presence of the immunosuppressant. A decrease in the immune response in the presence of an immunosuppressant is typically measured by a decrease in cytokine production (e.g., IFNα, IFNγ, TNFα, IL-1β, IL-2, IL-6, IL-8, IL-10, IL-12, or IL-18) by a responder cell in vitro or a decrease in cytokine production in the sera of a mammalian subject after administration of the interfering RNA.

As used herein, the term “responder cell” refers to a cell, preferably a mammalian cell, that produces a detectable immune response when contacted with an immunostimulatory therapeutic nucleic acid. Exemplary responder cells include, e.g., dendritic cells, macrophages, peripheral blood mononuclear cells (PBMCs), splenocytes, and the like. Exemplary responder cell can be human THP1 monocytes and murine RAW macrophage cells. Detectable immune responses can be readily measured in vitro by using various reporter constructs including interferon regulatory factor (IRF)-inducible reporter constructs using, e.g., THP1-Interferon stimulated gene (ISG) or RAW-ISG cells. In vivo immune responses can be measured by determining production levels of cytokines or growth factors such as TNF-α, IFN-α, IFN-β, IFN-γ, IL-1α, IL-10, IL-2, IL-3, IL-4, IL-5, IL-6, IL-8, IL-10, IL-12, IL-18, IP-10, TGF, VEGF, VEGFR or combinations thereof. Further, immune responses can be also measured by detecting levels of chemokine such as MCP-1, MIP-1α (CCL3), MIP-1β (CCL4), and Rantes (CCL5).

As used herein, the term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

As used herein, the term “lipid particle” includes a lipid formulation that can be used to deliver a therapeutic agent such as nucleic acid therapeutics and/or an immunosuppressant to a target site of interest (e.g., cell, tissue, organ, and the like). In preferred embodiments, the lipid particle of the invention is a nucleic acid containing lipid particle, which is typically formed from a cationic lipid, a non-cationic lipid, and optionally a conjugated lipid that prevents aggregation of the particle.

In other preferred embodiments, a therapeutic agent such as a therapeutic nucleic acid may be encapsulated in the lipid portion of the particle, thereby protecting it from enzymatic degradation. In other preferred embodiments, an immunosuppressant can be optionally included in the nucleic acid containing lipid particles.

As used herein, the term “lipid encapsulated” can refer to a lipid particle that provides an active agent or therapeutic agent, such as a nucleic acid (e.g., a ceDNA), with full encapsulation, partial encapsulation, or both. In a preferred embodiment, the nucleic acid is fully encapsulated in the lipid particle (e.g., to form a nucleic acid containing lipid particle).

As used herein, the term “lipid conjugate” refers to a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipids described above can be mixed with other lipids including triglycerides and sterols.

As used herein, the term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

As used herein, the term “non-cationic lipid” refers to any amphipathic lipid as well as any other neutral lipid or anionic lipid.

As used herein, the term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

As used herein, the term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacyloxy-3-aminopropane, and 1,2-dialkyl-3-aminopropane.

As used herein, the term “aqueous solution” refers to a composition comprising in whole, or in part, water.

As used herein, the term “organic lipid solution” refers to a composition comprising in whole, or in part, an organic solvent having a lipid.

As used herein, the term “systemic delivery” refers to delivery of lipid particles that leads to a broad biodistribution of an active agent such as an interfering RNA (e.g., siRNA) within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

As used herein, the term “local delivery” refers to delivery of an active agent such as an interfering RNA (e.g., siRNA) directly to a target site within an organism. For example, an agent can be locally delivered by direct injection into a disease site such as a tumor or other target site such as a site of inflammation or a target organ such as the liver, heart, pancreas, kidney, and the like.

As used herein, the term “ceDNA” refers to capsid-free closed-ended linear double stranded (ds) duplex DNA for non-viral gene transfer, synthetic or otherwise. Detailed description of ceDNA is described in International application of PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International applications PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire content of which is incorporated herein by reference.

As used herein, the term “closed-ended DNA vector” refers to a capsid-free DNA vector with at least one covalently closed end and where at least part of the vector has an intramolecular duplex structure.

As used herein, the terms “ceDNA vector” and “ceDNA” are used interchangeably and refer to a closed-ended DNA vector comprising at least one terminal palindrome. In some embodiments, the ceDNA comprises two covalently-closed ends.

As used herein, the term “gap” is meant to refer to a discontinued portion of synthetic DNA vector of the present invention, creating a stretch of single stranded DNA portion in otherwise double stranded ceDNA. The gap can be 1 base-pair to 100 base-pair long in length in one strand of a duplex DNA. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 bp long in length. Exemplified gaps in the present disclosure can be 1 bp to 10 bp long, 1 to 20 bp long, 1 to 30 bp long in length.

As used herein, the term “nick” refers to a discontinuity in a double stranded DNA molecule where there is no phosphodiester bond between adjacent nucleotides of one strand typically through damage or enzyme action. It is understood that one or more nicks allow for the release of torsion in the strand during DNA replication and that nicks are also thought to play a role in facilitating binding of transcriptional machinery.

As used herein, the term “neDNA”, “nicked ceDNA” refers to a closed-ended DNA having a nick or a gap of 1-100 base pairs a stem region or spacer region upstream of an open reading frame (e.g., a promoter and transgene to be expressed).

As used herein, the term “terminal repeat” or “TR” includes any viral or non-viral terminal repeat or synthetic sequence that comprises at least one minimal required origin of replication and a region comprising a palindromic hairpin structure. A Rep-binding sequence (“RBS” or also referred to as Rep-binding element (RBE)) and a terminal resolution site (“TRS”) together constitute a “minimal required origin of replication” for an AAV and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs plays a critical role in mediating replication, viral particle and DNA packaging, DNA integration and genome and provirus rescue. TRs that are not inverse complement (palindromic) across their full length can still perform the traditional functions of ITRs, and thus, the term ITR is used to refer to a TR in an viral or non-viral AAV vector that is capable of mediating replication of in the host cell. It will be understood by one of ordinary skill in the art that in a complex AAV vector configurations more than two ITRs or asymmetric ITR pairs may be present.

The “ITR” can be artificially synthesized using a set of oligonucleotides comprising one or more desirable functional sequences (e.g., palindromic sequence, RBS). The ITR sequence can be an AAV ITR, an artificial non-AAV ITR, or an ITR physically derived from a viral AAV ITR (e.g., ITR fragments removed from a viral genome). For example, the ITR can be derived from the family Parvovirfdae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. Typically, ITR sequences can be derived not only from AAV, but also from Parvovirus, lentivirus, goose virus, B19, in the configurations of wildtype, “doggy bone” and “dumbbell shape”, symmetrical or even asymmetrical ITR orientation. Although the ITRs are typically present in both 5′ and 3′ ends of an AAV vector, ITR can be present in only one of end of the linear vector. For example, the ITR can be present on the 5′ end only. Some other cases, the ITR can be present on the 3′ end only in synthetic AAV vector. For convenience herein, an ITR located 5′ to (“upstream of”) an expression cassette in a synthetic AAV vector is referred to as a “5′ ITR” or a “left ITR”, and an ITR located 3′ to (“downstream of”) an expression cassette in a vector or synthetic AAV is referred to as a “3′ ITR” or a “right ITR”.

As used herein, a “wild-type ITR” or “WT-ITR” refers to the sequence of a naturally occurring ITR sequence in an AAV genome or other dependovirus that remains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompasses for use herein include WT-ITR sequences as result of naturally occurring changes (e.g., a replication error).

As used herein, the term “substantially symmetrical WT-ITRs” or a “substantially symmetrical WT-ITR pair” refers to a pair of WT-ITRs within a synthetic AAV vector that are both wild type ITRs that have an inverse complement sequence across their entire length. For example, an ITR can be considered to be a wild-type sequence, even if it has one or more nucleotides that deviate from the canonical naturally occurring canonical sequence, so long as the changes do not affect the physical and functional properties and overall three-dimensional structure of the sequence (secondary and tertiary structures). In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably and refer to an ITR with a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single synthetic AAV genome that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single stranded AAV genome that are wild-type or mutated (e.g., modified relative to wild-type) dependoviral ITR sequences and are inverse complements across their full length. In one non-limiting example, both ITRs are wild type ITRs sequences from AAV2. In another example, neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a synthetic AAV vector is referred to as a “5′ ITR” or a “left ITR”, and an ITR located 3′ to (downstream of) an expression cassette in a synthetic AAV vector is referred to as a “3′ ITR” or a “right ITR”.

As used herein, the terms “substantially symmetrical modified-ITRs” or a “substantially symmetrical mod-ITR pair” refers to a pair of modified-ITRs within a synthetic AAV that are both that have an inverse complement sequence across their entire length. For example, the a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

As used herein, the term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. In one embodiment, the term flanking refers to terminal repeats at each end of the linear single strand synthetic AAV vector.

As used herein, the term “spacer region” refers to an intervening sequence that separates functional elements in a vector or genome. In some embodiments, AAV spacer regions keep two functional elements at a desired distance for optimal functionality. In some embodiments, the spacer regions provide or add to the genetic stability of the vector or genome. In some embodiments, spacer regions facilitate ready genetic manipulation of the genome by providing a convenient location for cloning sites and a gap of design number of base pair. For example, in certain aspects, an oligonucleotide “polylinker” or “poly cloning site” containing several restriction endonuclease sites, or a non-open reading frame sequence designed to have no known protein (e.g., transcription factor) binding sites can be positioned in the vector or genome to separate the cis-acting factors, e.g., inserting a 6mer, 12mer, 18mer, 24mer, 48mer, 86mer, 176mer, etc., for example, between the terminal resolution site and the upstream transcriptional regulatory element as in an AAV vector or genome.

As used herein, the terms “Rep binding site” (“RBS”) and “Rep binding element” (“RBE”) are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are well known in the art, and include, for example, 5′-GCGCGCTCGCTCGCTC-3′(SEQ ID NO: 1), an RBS sequence identified in AAV2. Any known RBS sequence may be used in the embodiments of the invention, including other known AAV RBS sequences and other naturally known or synthetic RBS sequences. Without being bound by theory it is thought that the nuclease domain of a Rep protein binds to the duplex nucleotide sequence GCTC, and thus the two known AAV Rep proteins bind directly to and stably assemble on the duplex oligonucleotide, 5′-(GCGCXGCTCXGCTCXGCTC)-3′ (SEQ ID NO: 1). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “trs” are used interchangeably herein and refer to a region at which Rep forms a tyrosine-phosphodiester bond with the 5′ thymidine generating a 3′-OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′ (SEQ ID NO: ), the hexanucleotide sequence identified in AAV2. Any known TRS sequence may be used in the embodiments of the invention, including other known AAV TRS sequences and other naturally known or synthetic TRS sequences such as AGTT, GGTTGG, AGTTGG, AGTTGA, and other motifs such as RRTRR.

As used herein, the terms “sense” and “antisense” refer to the orientation of the structural element on the polynucleotide. The sense and antisense versions of an element are the reverse complement of each other.

As used herein, the term “synthetic AAV vector” and “synthetic production of AAV vector” refers to an AAV vector and synthetic production methods thereof in an entirely cell-free environment.

As used herein, the term “reporter” refers to a protein that can be used to provide a detectable read-out. A reporter generally produces a measurable signal such as fluorescence, color, or luminescence. Reporter protein coding sequences encode proteins whose presence in the cell or organism is readily observed. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest. Promoters are regions of nucleic acid that initiate transcription of a particular gene. Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to, homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

As used herein, the term “promoter” refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the synthetic AAV vectors disclosed herein. A promoter sequence may be bounded at its 3′ terminus by the transcription initiation site and extends upstream (5′ direction) to include the minimum number of bases or elements necessary to initiate transcription at levels detectable above background.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

As used herein, the terms “expression cassette” and “expression unit” are used interchangeably and refer to a heterologous DNA sequence that is operably linked to a promoter or other DNA regulatory sequence sufficient to direct transcription of a transgene of a DNA vector, e.g., synthetic AAV vector. Suitable promoters include, for example, tissue specific promoters. Promoters can also be of AAV origin.

As used herein, the term “operably linked” refers to a juxtaposition wherein the components so described are in a relationship permitting them to function in their intended manner. For instance, a promoter is operably linked to a coding sequence if the promoter affects its transcription or expression. A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

As used herein, the terms “DNA regulatory sequences,” “control elements,” and “regulatory elements” are used interchangeably to refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence. In some embodiments, a coding nucleic acid segment is positioned under the control of a “recombinant promoter” or “heterologous promoter,” both of which refer to a promoter that is not normally associated with the encoded nucleic acid sequence that it is operably linked to in its natural environment. Similarly, a “recombinant or heterologous enhancer” refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference in its entirety). Furthermore, it is contemplated that control sequences that direct transcription and/or expression of sequences within non-nuclear organelles such as mitochondria, chloroplasts, and the like, can be employed as well.

As used herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

As used herein, the term “subject” refers to a human or animal, to whom treatment, including prophylactic treatment, with the therapeutic nucleic acid according to the present invention, is provided. Usually the animal is a vertebrate such as, but not limited to a primate, rodent, domestic animal or game animal. Primates include but are not limited to, chimpanzees, cynomolgus monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the term “host cell” includes any cell type that is susceptible to transformation, transfection, transduction, and the like with nucleic acid therapeutics of the present disclosure. As non-limiting examples, a host cell can be an isolated primary cell, pluripotent stem cells, CD34⁺ cells, induced pluripotent stem cells, or any of a number of immortalized cell lines (e.g., HepG2 cells). Alternatively, a host cell can be an in situ or in vivo cell in a tissue, organ or organism. Furthermore, a host cell can be a target cell of, for example, a mammalian subject (e.g., human patient in need of gene therapy).

As used herein, the term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

As used herein, the term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

As used herein, the term “homology” or “homologous” as used herein is defined as the percentage of nucleotide residues in the homology arm that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm of a repair template, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

As used herein, the terms “heterologous nucleotide sequence” and “transgene” are used interchangeably and refer to a nucleic acid of interest (other than a nucleic acid encoding a capsid polypeptide) that is incorporated into and may be delivered and expressed by a vector, such as ceDNA vector, as disclosed herein. A heterologous nucleic acid sequence may be linked to a naturally occurring nucleic acid sequence (or a variant thereof) (e.g., by genetic engineering) to generate a chimeric nucleotide sequence encoding a chimeric polypeptide. A heterologous nucleic acid sequence may be linked to a variant polypeptide (e.g., by genetic engineering) to generate a nucleotide sequence encoding a fusion variant polypeptide.

As used herein, a “vector” or “expression vector” is a replicon, such as plasmid, bacmid, phage, virus, virion, or cosmid, to which another DNA segment, i.e. an “insert” “transgene” or “expression cassette”, may be attached so as to bring about the expression or replication of the attached segment (“expression cassette”) in a cell. A vector can be a nucleic acid construct designed for delivery to a host cell or for transfer between different host cells. As used herein, a vector can be viral or non-viral in origin in the final form. However, for the purpose of the present disclosure, a “vector” generally refers to synthetic AAV vector or a nicked ceDNA vector. Accordingly, the term “vector” encompasses any genetic element that is capable of replication when associated with the proper control elements and that can transfer gene sequences to cells. In some embodiments, a vector can be a recombinant vector or an expression vector.

As used herein, the phrase “recombinant vector” means a vector that includes a heterologous nucleic acid sequence, or “transgene” that is capable of expression in vivo. It is to be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the term “expression vector” refers to a vector that directs expression of an RNA or polypeptide from sequences linked to transcriptional regulatory sequences on the vector. The sequences expressed will often, but not necessarily, be heterologous to the host cell. An expression vector may comprise additional elements, for example, the expression vector may have two replication systems, thus allowing it to be maintained in two organisms, for example in human cells for expression and in a prokaryotic host for cloning and amplification. the expression vector may be a recombinant vector.

As used herein, the term “expression” refers to the cellular processes involved in producing RNA and proteins and as appropriate, secreting proteins, including where applicable, but not limited to, for example, transcription, transcript processing, translation and protein folding, modification and processing. As used herein, the phrase “expression products” include RNA transcribed from a gene (e.g., transgene), and polypeptides obtained by translation of mRNA transcribed from a gene.

As used herein, the term “gene” means the nucleic acid sequence which is transcribed (DNA) to RNA in vitro or in vivo when operably linked to appropriate regulatory sequences.

As used herein, the phrase “genetic disease” or “genetic disorder” refers to a disease, partially or completely, directly or indirectly, caused by one or more abnormalities in the genome, including and especially a condition that is present from birth. The abnormality may be a mutation, an insertion or a deletion in a gene. The abnormality may affect the coding sequence of the gene or its regulatory sequence. The genetic disease may be, but is not limited to, phenylketonuria (PKU), sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, and galactosialidosis. Also included in genetic disorders are amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis (LCA, e.g., LCA10 [CEP290]), Stargardt macular dystrophy (ABCA4), omithine transcarbamylase (OTC) deficiency, Usher syndrome, alpha-1 antitrypsin deficiency, progressive familial intrahepatic cholestasis (PFIC), and Cathepsin A deficiency.

As used herein the term “comprising” or “comprises” is used in reference to compositions, methods, processes, and respective component(s) thereof, that are essential to the processes, methods or compositions, yet open to the inclusion of unspecified elements, whether essential or not. The use of “comprising” indicates inclusion rather than limitation.

As used herein, the term “consisting of” refers to compositions, methods, processes, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below.

The abbreviation, “e.g.” is derived from the Latin exempli gratia and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean ±1%. The present invention is further explained in detail by the following examples, but the scope of the invention should not be limited thereto.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this invention is not limited in any manner to the particular methodology, protocols, and reagents, etc., described herein and as such can vary. The terminology used herein is for the purpose of describing particular embodiments only and is not intended to limit the scope of the present invention, which is defined solely by the claims.

### II. Therapeutic Nucleic Acids (TNAs)

Nucleic acids are large, highly charged, rapidly degraded and cleared from the body, and offer generally poor pharmacological properties because they are recognized as a foreign matter to the body and become a target of an innate immune response. Hence, certain therapeutic nucleic acid (TNA) (e.g., antisense oligonucleotide or viral vectors) can often trigger immune responses in vivo. The present disclosure provides pharmaceutical compositions and methods that may ameliorate, reduce or eliminate such immune responses and enhance efficacy of therapeutic nucleic acids by increasing expression levels through maximizing the durability of the therapeutic nucleic acid in a reduced immune-responsive state in a subject recipient. This may also minimize any potential adverse events that may lead to an organ damage or other toxicity in the course of gene therapy. Many of the compositions and methods provided herein relate to the administration of a specific immunosuppressant in conjunction with a therapeutic nucleic acid, thereby reducing the innate immune response triggered by the presence of the TNA, which may cause or aggravate adverse events through detrimental inflammatory reactions in the subject.

The immunogenic/immunostimulatory nucleic acids can include both deoxyribonucleic acids and ribonucleic acids. For deoxyribonucleic acids (DNA), a particular sequence or motif has been shown to induce immune stimulation in mammals. These sequence or motifs include, but are not limited to, CpG motifs, pyrimidine-rich sequences, and palindrome sequences. CpG motifs in deoxyribonucleic acid are often recognized by the endosomal toll-like receptor 9 (TLR-9) which, in turn, triggers both the innate immune stimulatory pathway and the acquired immune stimulatory pathway. Certain immunostimulatory ribonucleic acid (RNA) sequences bind to toll-like receptor 6 and 7 (TLR-6 and TLR-7) and are believed to activate proinflammatory response through the innate immune responses. Furthermore, double-stranded RNA can be often immunostimulatory because of its binding to TLR-3. Therefore, foreign nucleic acid molecules, either pathogen derived or therapeutic in their origin, can be highly immunogenic in vivo.

The characterization and development of nucleic acid molecules for potential therapeutic use in conjunction with antagonists of IFN signaling and synthesis pathways are provided herein. In some embodiments, chemical modification of oligonucleotides for the purpose of altered and improved in vivo properties (delivery, stability, life-time, folding, target specificity), as well as their biological function and mechanism that directly correlate with therapeutic application, are described where appropriate.

Illustrative therapeutic nucleic acids of the present disclosure that can be immunostimulatory and require use of immunosuppressants disclosed herein can include, but are not limited to, minigenes, plasmids, minicircles, small interfering RNA (siRNA), microRNA (miRNA), antisense oligonucleotides (ASO), ribozymes, closed ended double stranded DNA (e.g., ceDNA, CELiD, linear covalently closed DNA (“ministring”), doggybone (dbDNA™), protelomere closed ended DNA, or dumbbell linear DNA), dicer-substrate dsRNA, small hairpin RNA (shRNA), asymmetrical interfering RNA (aiRNA), mricroRNS (miRNA), mRNA, tRNA, rRNA, and DNA viral vectors, viral RNA vector, and any combination thereof.

siRNA or miRNA that can downregulate the intracellular levels of specific proteins through a process called RNA interference (RNAi) are also contemplated by the present invention to be nucleic acid therapeutics. After siRNA or miRNA is introduced into the cytoplasm of a host cell, these double-stranded RNA constructs can bind to a protein called RISC. The sense strand of the siRNA or miRNA is removed by the RISC complex. The RISC complex, when combined with the complementary mRNA, cleaves the mRNA and release the cut strands. RNAi is by inducing specific destruction of mRNA that results in downregulation of a corresponding protein.

Antisense oligonucleotides (ASO) and ribozymes that inhibit mRNA translation into protein can be nucleic acid therapeutics. For antisense constructs, these single stranded deoxy nucleic acids have a complementary sequence to the sequence of the target protein mRNA, and Watson—capable of binding to the mRNA by Crick base pairing. This binding prevents translation of a target mRNA, and/or triggers RNaseH degradation of the mRNA transcript. As a result, the antisense oligonucleotide has increased specificity of action (i.e., down-regulation of a specific disease-related protein).

In any of the methods provided herein, the therapeutic nucleic acid can be a therapeutic RNA. Said therapeutic RNA can be an inhibitor of mRNA translation, agent of RNA interference (RNAi), catalytically active RNA molecule (ribozyme), transfer RNA (tRNA) or an RNA that binds an mRNA transcript (ASO), protein or other molecular ligand (aptamer). In any of the methods provided herein, the agent of RNAi can be a double-stranded RNA, single-stranded RNA, micro RNA, short interfering RNA, short hairpin RNA, or a triplex-forming oligonucleotide.

According to some embodiments, the therapeutic nucleic acid is a closed ended double stranded DNA, e.g., a ceDNA. According to some embodiments, the expression and/or production of a therapeutic protein in a cell is from a non-viral DNA vector, e.g., a ceDNA vector. A distinct advantage of ceDNA vectors for expression of a therapeutic protein over traditional AAV vectors, and even lentiviral vectors, is that there is no size constraint for the heterologous nucleic acid sequences encoding a desired protein. Thus, even a large therapeutic protein can be expressed from a single ceDNA vector. Thus, ceDNA vectors can be used to express a therapeutic protein in a subject in need thereof.

In general, a ceDNA vector for expression of a therapeutic protein as disclosed herein, comprises in the 5′ to 3′ direction: a first adeno-associated virus (AAV) inverted terminal repeat (ITR), a nucleotide sequence of interest (for example an expression cassette as described herein) and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

### III. Closed Ended DNA (ceDNA) Vectors

Described herein are novel non-viral, capsid-free DNA vectors with covalently-closed ends (ceDNA) administered in a composition in conjunction with one or more tyrosine kinase inhibitors (TKIs). The non-viral capsid free DNA vectors are produced in permissive host cells from an expression construct (e.g., a plasmid, a Bacmid, a baculovirus, or an integrated cell-line) containing a heterologous nucleic acid, e.g. a transgene positioned between two inverted terminal repeat (ITR) sequences. In some embodiments, at least one of the ITRs is modified by deletion, insertion, and/or substitution as compared to a wild-type ITR sequence (e.g. AAV ITR); and at least one of the ITRs comprises a functional terminal resolution site (TRS) and a Rep binding site. In one embodiment, at least one of the ITRs has at least one polynucleotide deletion, insertion, or substitution with respect to a corresponding AAV ITR to induce replication of the DNA vector in a host cell in the presence of Rep protein. As discussed above, it is envisioned that any ITR can be used. For exemplary purposes, the ITRs in the ceDNA constructs contemplated herein are a modified ITR and a WT ITR and are an example of an asymmetric ITR pair. However, encompassed herein are ceDNA vectors that contain a heterologous nucleic acid sequence (e.g., a transgene) positioned between two inverted terminal repeat (ITR) sequences, where the ITR sequences can be an asymmetrical ITR pair or a symmetrical- or substantially symmetrical ITR pair, as these terms are defined herein. A ceDNA vector comprising a NLS as disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

In some embodiments, the methods and compositions described herein relate to the use of one or more TKIs with any ceDNA vector, including but not limited to, a ceDNA vector comprising asymmetric ITRS as disclosed in International Patent Application PCT/US18/49996, filed on Sep. 7, 2018 (see, e.g., Examples 1-4); a ceDNA vector for gene editing as disclosed on the International Patent Application PCT/US18/64242 filed on Dec. 6, 2018 (see, e.g., Examples 1-7), or a ceDNA vector for production of antibodies or fusion proteins, as disclosed in the International Patent Application PCT/US19/18016, filed on Feb. 14, 2019, (e.g., see Examples 1-4), or a ceDNA vector for controlled transgene expression, as disclosed in International Patent Application PCT/US19/18927 filed on Feb. 22, 2019, each of which are incorporated herein in their entirety by reference. In some embodiments, it is also envisioned that the methods and compositions described herein can be used with a synthetically produced ceDNA vector, e.g., a ceDNA vector produced in a cell free or insect-free system of ceDNA production, as disclosed in International Application PCT/US19/14122, filed on Jan. 18, 2019, incorporated by reference in its entirety herein.

The ceDNA vector is preferably duplex, or self-complementary, over at least a portion of the molecule, e.g. the transgene. The ceDNA vector has covalently closed ends, and thus is preferably resistant to exonuclease digestion (e.g. Exo I or Exo III) for over an hour at 37° C. The presence of Rep protein in the host cells (e.g. insect cells or mammalian cells) promotes replication of the ceDNA vector polynucleotide template that has the modified ITR inducing production of non-viral capsid free DNA vector with covalently closed ends. The covalently closed ended molecule continues to accumulate in permissive cells through replication and is preferably sufficiently stable over time in the presence of Rep protein under standard replication conditions, e.g., to accumulate at yields of at least 1 pg/cell, preferably at least 2 pg/cell, preferably at least 3 pg/cell, more preferably at least 4 pg/cell, even more preferably at least 5 pg/cell.

In some embodiments, DNA vectors are produced by providing cells (e.g. insect cells or mammalian cells e.g. 293 cells etc.) harboring a polynucleotide vector template (e.g., expression construct) that comprises two different ITRs (e.g. AAV ITRs) and a nucleotide sequence of interest (a heterologous nucleic acid, expression cassette) positioned between the ITRs, wherein at least one of the ITRs is a modified ITR comprising an insertion, substitution, or deletion relative to the other ITR. The polynucleotide vector template described herein contains at least one functional ITR that comprises a Rep-binding site (RBS; e.g. 5′-GCGCGCTCGCTCGCTC-3′ (SEQ ID NO: 1) for AAV2) and a functional terminal resolution site (TRS; e.g. 5′-AGTT). The cells do not express viral capsid proteins and the polynucleotide vector template is devoid of viral capsid coding sequences.

In the presence of Rep, the vector polynucleotide template having at least one modified ITR replicates to produce ceDNA. The ceDNA production undergoes two steps: first, excision (“rescue”) of template from the vector backbone (e.g. plasmid, bacmid, genome etc.) via Rep proteins, and second, Rep mediated replication of the excised vector genome. Rep proteins and Rep binding sites of the various AAV serotypes are well known to those of skill in the art One of skill in the art understands to choose a Rep protein from a serotype that binds to and replicates the functional ITR.

The cells harboring the vector polynucleotide either already contain Rep (e.g. a cell line with inducible rep), or are transduced with a vector that contains Rep and are then grown under conditions permitting replication and release of ceDNA vector. The ceDNA vector DNA is then harvested and isolated from the cells. The presence of the capsid-free, non-viral DNA ceDNA vector can be confirmed by digesting the vector DNA isolated from the cells with a restriction enzyme having a single recognition site on the DNA vector and analyzing the digested DNA material on a non-denaturing gel to confirm the presence of characteristic bands of linear and continuous DNA as compared to linear and non-continuous DNA.

The vector polynucleotide expression template (e.g. TTX-plasmid, Bacmid etc.), and/or ii) a polynucleotide that encodes Rep can be introduced into cells using any means well known to those of skill in the art, including but not limited to transfection (e.g. calcium phosphate, nanoparticle, or liposome), or introduction by viral vectors, e.g. HSV or baculovirus. In one embodiment, the TTX-plasmid comprises a restriction cloning site operably positioned between the ITRs where the heterologous nucleic acid (e.g. expression cassette comprising a reporter gene or a therapeutic nucleic acid) can be inserted.

In some embodiments, the host cells used to make the ceDNA vectors described herein are insect cells. In another preferred embodiment, baculovirus is used to deliver both the polynucleotide that encodes Rep protein and the non-viral DNA vector polynucleotide expression construct template for ceDNA. Examples of such processes for obtaining and isolating ceDNA vectors are described in Example 1.

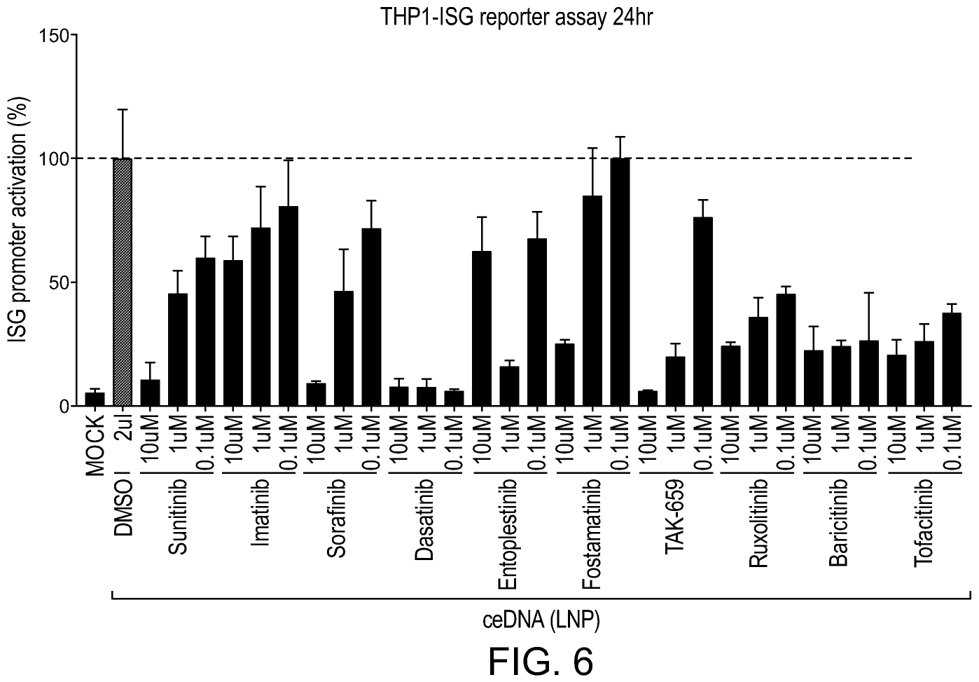

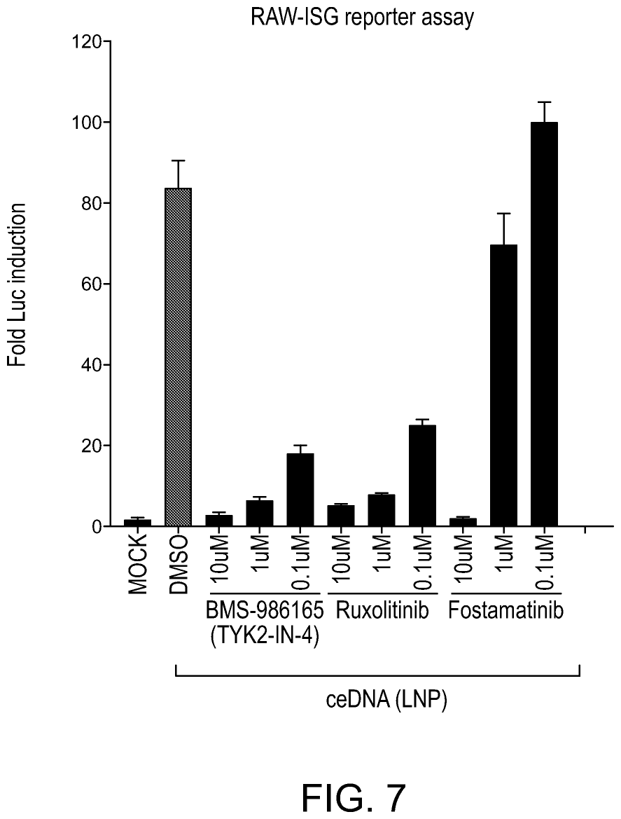

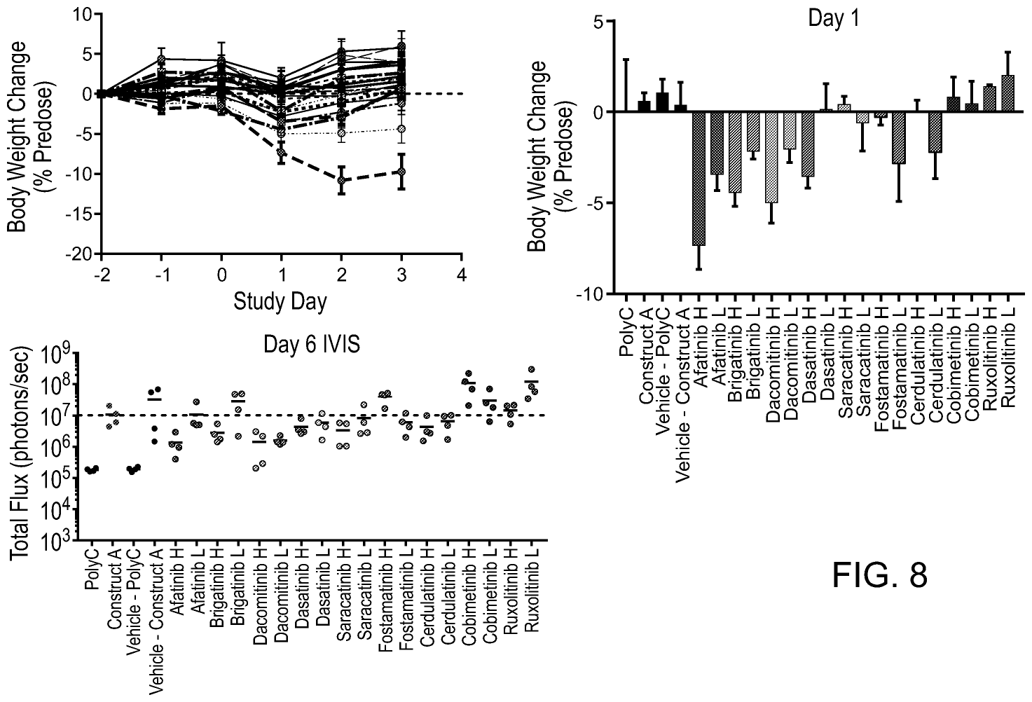

According to some embodiments, synthetic ceDNA is produced via excision from a double-stranded DNA molecule. Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. In some embodiments, the double stranded DNA construct is a ceDNA plasmid, e.g., see, e.g., FIG. 6 in International patent application PCT/US2018/064242, filed Dec. 6, 2018).

In some embodiments, a construct to make a ceDNA vector (e.g., a synthetic AAV vector) comprises additional components to regulate expression of the transgene, for example, regulatory switches, to regulate the expression of the transgene, or a kill switch, which can kill a cell comprising the vector.

A molecular regulatory switch is one which generates a measurable change in state in response to a signal. Such regulatory switches can be usefully combined with the ceDNA vectors described herein to control the output of expression of the transgene. In some embodiments, the ceDNA vector comprises a regulatory switch that serves to fine tune expression of the transgene. For example, it can serve as a biocontainment function of the ceDNA vector. In some embodiments, the switch is an “ON/OFF” switch that is designed to start or stop (i.e., shut down) expression of the gene of interest in the ceDNA vector in a controllable and regulatable fashion. In some embodiments, the switch can include a “kill switch” that can instruct the cell comprising the synthetic ceDNA vector to undergo cell programmed death once the switch is activated. Exemplary regulatory switches encompassed for use in a ceDNA vector can be used to regulate the expression of a transgene, and are more fully discussed in International application PCT/US18/49996, which is incorporated herein in its entirety by reference and described herein.

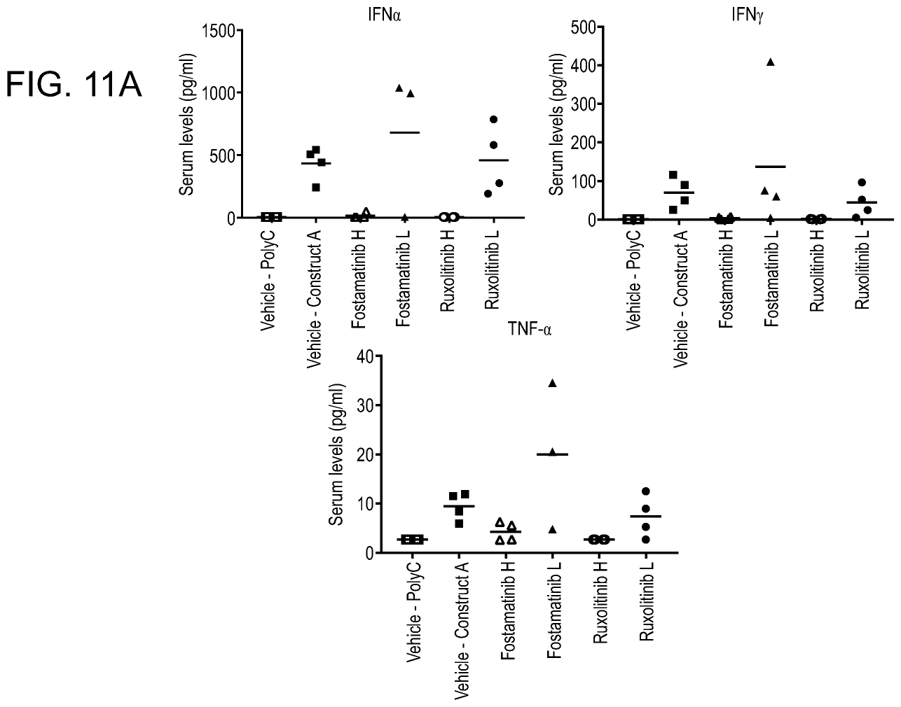

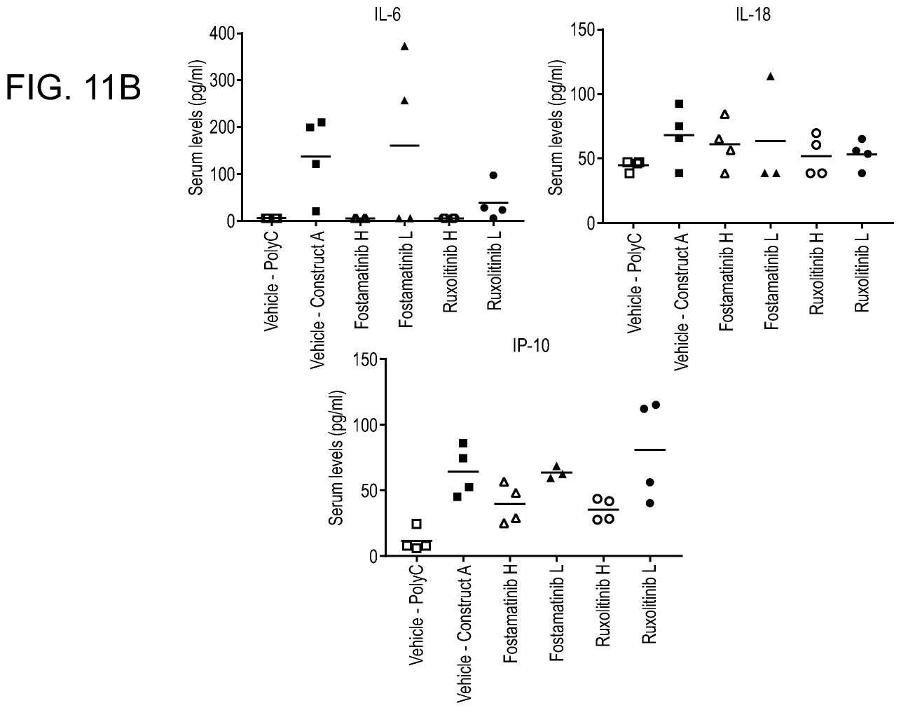

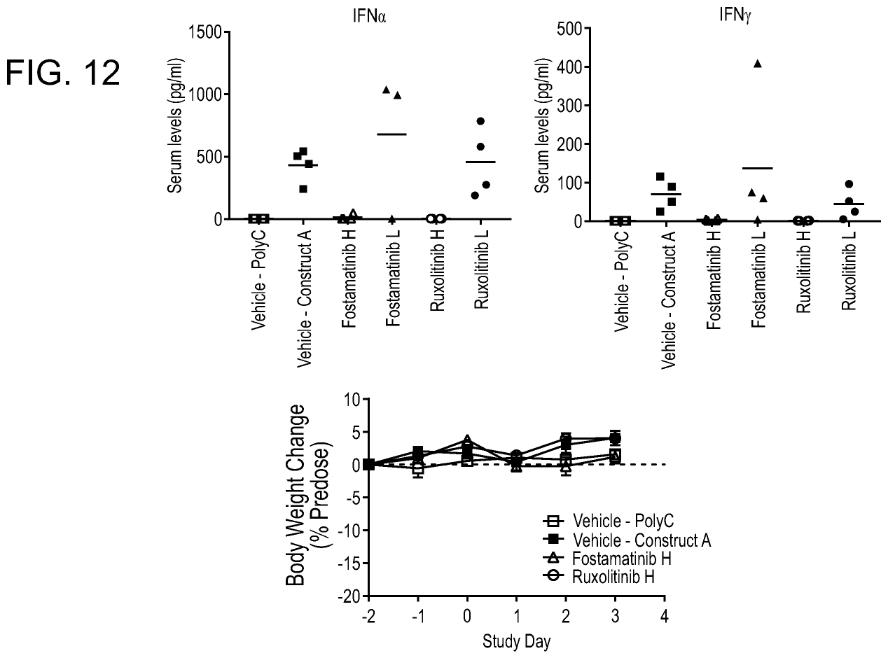

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122, incorporated by reference in its entirety herein, shows an exemplary method of ligating a 5′ ITR oligonucleotide and a 3′ ITR oligonucleotide to a double stranded polynucleotide comprising an expression cassette.

An exemplary method of producing a ceDNA vector using a synthetic method is provided in Example 4 of PCT/US19/14122, incorporated by reference in its entirety herein, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

In yet another aspect, the invention provides for host cell lines that have stably integrated the DNA vector polynucleotide expression template (ceDNA template) described herein, into their own genome for use in production of the non-viral DNA vector. Methods for producing such cell lines are described in Lee, L. et al. (2013) Plos One 8(8): e69879, which is herein incorporated by reference in its entirety. Preferably, the Rep protein (e.g. as described in Example 1) is added to host cells at an MOI of 3. In one embodiment, the host cell line is an invertebrate cell line, preferably insect Sf9 cells. When the host cell line is a mammalian cell line, preferably 293 cells the cell lines can have polynucleotide vector template stably integrated, and a second vector, such as herpes virus can be used to introduce Rep protein into cells, allowing for the excision and amplification of ceDNA in the presence of Rep.

Any promoter can be operably linked to the heterologous nucleic acid (e.g. reporter nucleic acid or therapeutic transgene) of the vector polynucleotide. The expression cassette can contain a synthetic regulatory element, such as CAG promoter. The CAG promoter comprises (i) the cytomegalovirus (CMV) early enhancer element, (ii) the promoter, the first exon and the first intron of the chicken beta actin gene, and (ii) the splice acceptor of the rabbit beta globin gene. Alternatively, expression cassette can contain an Alpha-1-antitrypsin (AAT) promoter, a liver specific (LP1) promoter, or Human elongation factor-1 alpha (EF1-α) promoter. In some embodiments, the expression cassette includes one or more constitutive promoters, for example, the retroviral Rous sarcoma virus (RSV) LTR promoter (optionally with the RSV enhancer), cytomegalovirus (CMV) immediate early promoter (optionally with the CMV enhancer). Alternatively, an inducible or repressible promoter, a native promoter for a transgene, a tissue-specific promoter, or various promoters known in the art can be used. Suitable transgenes for gene therapy are well known to those of skill in the art.

The capsid-free ceDNA vectors can also be produced from vector polynucleotide expression constructs that further comprise cis-regulatory elements, or combination of cis regulatory elements, a non-limiting example include a woodchuck hepatitis virus posttranscriptional regulatory element (WPRE) and BGH polyA, or e.g. beta-globin polyA. Other posttranscriptional processing elements include, e.g. the thymidine kinase gene of herpes simplex virus, or hepatitis B virus (HBV). The expression cassettes can include any poly-adenylation sequence known in the art or a variation thereof, such as a naturally occurring isolated from bovine BGHpA or a virus SV40 pA, or synthetic. Some expression cassettes can also include SV40 late polyA signal upstream enhancer (USE) sequence. The, USE can be used in combination with SV40 pA or heterologous poly-A signal.

The time for harvesting and collecting DNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce DNA-vectors) but before the majority of cells start to die because of the viral toxicity. The DNA-vectors can be isolated using plasmid purification kits such as Qiagen Endo-Free Plasmid kits. Other methods developed for plasmid isolation can be also adapted for DNA-vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

In one embodiment, the capsid free non-viral DNA vector comprises or is obtained from a plasmid comprising a polynucleotide template comprising in this order: a first adeno-associated virus (AAV) inverted terminal repeat (ITR), a nucleotide sequence of interest (for example an expression cassette of an exogenous DNA) and a modified AAV ITR, wherein said template nucleic acid molecule is devoid of AAV capsid protein coding. In a further embodiment, the nucleic acid template of the invention is devoid of viral capsid protein coding sequences (i.e. it is devoid of AAV capsid genes but also of capsid genes of other viruses). In addition, in a particular embodiment, the template nucleic acid molecule is also devoid of AAV Rep protein coding sequences. Accordingly, in a preferred embodiment, the nucleic acid molecule of the invention is devoid of both functional AAV cap and AAV rep genes.

In one embodiment, ceDNA can include an ITR structure that is mutated with respect to the wild type AAV2 ITR disclosed herein, but still retains an operable RBE, TRS and RBE′ portion.

Encompassed herein are methods and compositions comprising the ceDNA vector for therapeutic protein production, which may further include a delivery system, such as but not limited to, liposome delivery systems.

Various techniques and methods are known in the art for delivering nucleic acids to cells. For example, a closed-ended DNA vector, including a ceDNA vector, as described herein can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering a closed-ended DNA vector, including a ceDNA vector, as described herein to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326, the contents of each of which are incorporated by reference in their entireties herein.

Nucleic acids and closed-ended DNA vector, including a ceDNA vector, as described herein can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TURBOFECT™ Transfection Reagent (Thermo Fisher Scientific®), Pro-Ject Reagent (Thermo Fisher Scientific®), TRANSPASST™ P Protein Transfection Reagent (New England Biolabs®), CHARIOT™ Protein Delivery Reagent (Active Motif), PROTEOJUICE™ Protein Transfection Reagent (EMD Millipore®), 293fectin, LIPOFECTAMINET™ 2000, LIPOFECTAMINET™ 3000 (Thermo Fisher Scientific®), LIPOFECTAMINE™ (Termo Fisher Scientific®), LIPOFECTIN™ (Thermo Fisher Scientific®), DMRIE-C, CELLFECTIN™ (Thermo Fisher Scientific®), OLIGOFECTAMINET™ (Termo Fisher Scientific®), LIPOFECTACE™, FUGENE™ (Roche®, Basel, Switzerland), FUGENET™ HD (Roche®), TRANSFECTAM™ (Transfectam, Promega®, Madison, Wis.), TFX-10™ (Promega®), TFX-20™ (Promega®), TFX-50™ (Promega), TRANSFECTIN™ (BioRad®, Hercules, Calif.), SILENTFECT™ (Bio-Rad®), EFFECTENET™ (Qiagen®, Valencia, Calif.), DC-chol (Avanti Polar Lipids), GENEPORTER™ (Gene Therapy Systems®, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dhannacon), ESCORT™ III (Sigma, St. Louis, Mo.), and ESCORT™ IV (Sigma® Chemical Co.). Nucleic acids, such as ceDNA, can also be delivered to a cell via microfluidics methods known to those of skill in the art.

A closed-ended DNA vector, including a ceDNA vector, as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a closed-ended DNA vector, including a ceDNA vector, as described herein can be delivered into hematopoietic stem cells, for example, by the methods as described, for example, in U.S. Pat. No. 5,928,638, incorporated by reference in its entirety herein.

A closed-ended DNA vector, including a ceDNA vector, as described herein can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids. Exemplary liposomes and liposome formulations are disclosed in International Application PCT/US2018/050042, filed on Sep. 7, 2018 and in International application PCT/US2018/064242, filed on Dec. 6, 2018, e.g., see the section entitled “Pharmaceutical Formulations”, the contents of each of which are incorporated by reference in their entireties herein.

Various delivery methods known in the art or modifications thereof can be used to deliver a closed-ended DNA vector, including a ceDNA vector, as described herein in vitro or in vivo. For example, in some embodiments, ceDNA vectors are delivered by making transient penetration in cell membrane by mechanical, electrical, ultrasonic, hydrodynamic, or laser-based energy so that DNA entrance into the targeted cells is facilitated. For example, a ceDNA vector can be delivered by transiently disrupting cell membrane by squeezing the cell through a size-restricted channel or by other means known in the art. In some cases, a ceDNA vector alone is directly injected as naked DNA into skin, thymus, cardiac muscle, skeletal muscle, or liver cells. In some cases, a ceDNA vector is delivered by gene gun. Gold or tungsten spherical particles (1-3 μm diameter) coated with capsid-free AAV vectors can be accelerated to high speed by pressurized gas to penetrate into target tissue cells.

Compositions comprising a closed-ended DNA vector, including a ceDNA vector, as described herein and a pharmaceutically acceptable carrier are specifically contemplated herein. In some embodiments, the ceDNA vector is formulated with a lipid delivery system, for example, liposomes as described herein. In some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods or ultrasound.

In some cases, a closed-ended DNA vector, including a ceDNA vector as described herein is delivered by hydrodynamic injection, which is a simple and highly efficient method for direct intracellular delivery of any water-soluble compounds and particles into internal organs and skeletal muscle in an entire limb.

In some cases, a closed-ended DNA vector, including a ceDNA vector, as described herein is delivered by ultrasound by making nanoscopic pores in membrane to facilitate intracellular delivery of DNA particles into cells of internal organs or tumors, so the size and concentration of the closed-ended DNA vector have a great role in efficiency of the system. In some cases, closed-ended DNA vectors, including a ceDNA vector, as described herein are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

### A. Exosomes:

In some embodiments, a closed-ended DNA vector, including a ceDNA vector, as described herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and 1 μm, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present invention.

### B. Microparticle/Nanoparticles:

In some embodiments, a closed-ended DNA vector, including a ceDNA vector and/or an immunosuppressant, as described herein is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristoylglycerol, PEG-DMG), for example as disclosed by Tam et al. (2013). Advances in Lipid Nanoparticles for siRNA delivery. Pharmaceuticals 5(3): 498-507.

In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some other embodiments, the lipid particles comprising a therapeutic nucleic acid and/or an immunosuppressant typically have a mean diameter of from about 20 nm to about 100 nm, 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm to ensure effective delivery. Nucleic acid containing lipid particles and their method of preparation are disclosed in, e.g., PCT/US18/50042, U.S. Patent Publication Nos. 20040142025 and 20070042031, the disclosures of which are herein incorporated by reference in their entirety for all purposes. In some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

According to some embodiments, a liquid pharmaceutical composition comprising a nucleic acid (e.g., a therapeutic nucleic acid, a nucleic acid used for research purposes) and/or inhibitor of the immune response (e.g., innate immune response) of the present invention may be formulated in lipid particles. In some embodiments, the lipid particle comprising a nucleic acid can be formed from a cationic lipid. In some other embodiments, the lipid particle comprising a nucleic acid can be formed from non-cationic lipid. In a preferred embodiment, the lipid particle of the invention is a nucleic acid containing lipid particle, which is formed from a cationic lipid comprising a nucleic acid selected from the group consisting of mRNA, antisense RNA and oligonucleotide, ribozymes, aptamer, interfering RNAs (RNAi), Dicer-substrate dsRNA, small hairpin RNA (shRNA), asymmetrical interfering RNA (aiRNA), microRNA (miRNA), minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone (dbDNA™) DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

Various lipid nanoparticles known in the art can be used to deliver a closed-ended DNA vector, including a ceDNA vector as described herein. For example, various delivery methods using lipid nanoparticles are described in U.S. Pat. Nos. 9,404,127, 9,006,417 and 9,518,272.

In some embodiments, a closed-ended DNA vector, including a ceDNA vector and/or inhibitor of the immune response (e.g., innate immune response), as described herein is delivered by a gold nanoparticle. Generally, a nucleic acid can be covalently bound to a gold nanoparticle or non-covalently bound to a gold nanoparticle (e.g., bound by a charge-charge interaction), for example as described by Ding et al. (2014). Gold Nanoparticles for Nucleic Acid Delivery. Mol. Ther. 22(6); 1075-1083. In some embodiments, gold nanoparticle-nucleic acid conjugates are produced using methods described, for example, in U.S. Pat. No. 6,812,334, the contents of which is incorporated by reference in its entirety herein.

### C. Conjugates

In some embodiments, a closed-ended DNA vector, including a ceDNA vector and/or inhibitor of the immune response (e.g., innate immune response), as described herein as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809.

In some embodiments, a closed-ended DNA vector, including a ceDNA vector and/or inhibitor of the immune response (e.g., innate immune response), as described herein as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309, incorporated by reference in its entirety herein. In some embodiments, a ceDNA vector as disclosed herein is conjugated to a poly(amide) polymer, for example as described by U.S. Pat. No. 8,987,377, incorporated by reference in its entirety herein. In some embodiments, a nucleic acid described by the disclosure is conjugated to a folic acid molecule as described in U.S. Pat. No. 8,507,455, incorporated by reference in its entirety herein.

In some embodiments, a closed-ended DNA vector, including a ceDNA vector and/or inhibitor of the immune response (e.g., innate immune response), as described herein as disclosed herein is conjugated to a carbohydrate, for example as described in U.S. Pat. No. 8,450,467, the contents of which is incorporated by reference in its entirety herein.

In some embodiments, the lipid nanoparticles may be conjugated with other moieties to prevent aggregation. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282, the contents of which is incorporated by reference in its entirety herein. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

### D. Nanocapsule

Alternatively, nanocapsule formulations of a closed-ended DNA vector, including a ceDNA vector and/or inhibitor of the immune response (e.g., innate immune response), as described herein as disclosed herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### E. Liposomes

A closed-ended DNA vector, including a ceDNA vector and/or inhibitor of the immune response (e.g., innate immune response), as described herein can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587, the contents of each of which are incorporated by reference in its entirety herein).

### F. Exemplary Liposome and Lipid Nanoparticle (LNP) Compositions

A closed-ended DNA vector, including a ceDNA vector and/or inhibitor of the immune response (e.g., innate immune response), as described herein can be added to liposomes for delivery to a cell, e.g., a cell in need of expression of the transgene. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

Lipid nanoparticles (LNPs) comprising ceDNA are disclosed in International Application PCT/US2018/050042, filed on Sep. 7, 2018, and International Application PCT/US2018/064242, filed on Dec. 6, 2018, which are each incorporated herein by reference in their entirety and envisioned for use in the methods and compositions as disclosed herein.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g. sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g. polyphosphate or sucrose octasulfate.

In some aspects, the disclosure provides for a lipid nanoparticle comprising a DNA vector, including a ceDNA vector as described herein and/or inhibitor of the immune response (e.g., innate immune response) and an ionizable lipid. For example, a lipid nanoparticle formulation that is made and loaded with ceDNA obtained by the process as disclosed in International Application PCT/US2018/050042, filed on Sep. 7, 2018, which is incorporated by reference in its entirety herein. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid particles are prepared at a total lipid to ceDNA (mass or weight) ratio of from about 10:1 to 30:1. In some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. According to some embodiments of any of the aspects or embodiments herein, the composition has a total lipid to ceDNA ratio of about 15:1. According to some embodiments of any of the aspects or embodiments herein, the composition has a total lipid to ceDNA ratio of about 30:1. According to some embodiments of any of the aspects or embodiments herein, the composition has a total lipid to ceDNA ratio of about 40:1. According to some embodiments of any of the aspects or embodiments herein, the composition has a total lipid to ceDNA ratio of about 50:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL

The ionizable lipid is typically employed to condense the nucleic acid cargo, e.g., ceDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

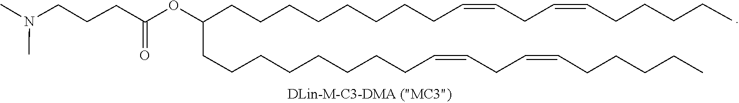 [SMILES: CCCCC/C=C\C/C=C\CCCCCCCCC(CCCCCCCC/C=C\C/C=C\CCC(C)CC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is the lipid ATX-002 as described in WO2015/074085, content of which is incorporated herein by reference in its entirety.

In some embodiments, the ionizable lipid is (13Z,16Z)—N,N-dimethyl-3-nonyldocosa-13,16-dien-1-amine (Compound 32), as described in WO2012/040184, content of which is incorporated herein by reference in its entirety.

In some embodiments, the ionizable lipid is Compound 6 or Compound 22 as described in WO2015/199952, content of which is incorporated herein by reference in its entirety.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. In some embodiments, ionizable lipid comprises from about 50 mol % to about 90 mol % of the total lipid present in the lipid nanoparticle.

In some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids envisioned for use in the methods and compositions comprising a DNA vector, including a ceDNA vector as described herein are described in International Application PCT/US2018/050042, filed on Sep. 7, 2018, and PCT/US2018/064242, filed on Dec. 6, 2018 which is incorporated herein in its entirety.

Exemplary non-cationic lipids are described in International application Publication WO2017/099823 and US patent publication US2018/0028664, the contents of both of which are incorporated herein by reference in their entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

In some embodiments, the lipid nanoparticles do not comprise any phospholipids. In some aspects, the lipid nanoparticle can further comprise a component, such as a sterol, to provide membrane integrity.

One exemplary sterol that can be used in the lipid nanoparticle is cholesterol and derivatives thereof. Exemplary cholesterol derivatives are described in International application WO2009/127060 and US patent publication US2010/0130588, contents of both of which are incorporated herein by reference in their entirety.

The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle.

In some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a PEGylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In some embodiments, a PEG-lipid is a compound disclosed in US2018/0028664, the content of which is incorporated herein by reference in its entirety.

In some embodiments, a PEG-lipid is disclosed in US20150376115 or in US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some examples, the PEG-lipid can be selected from the group consisting of PEG-DMG, 1,2-dimyristoyl-sn-glycero-3-phosphoethanolamine-N-[methoxy(polyethylene glycol)-2000].

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International patent application publications WO1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US patent application publications US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and US patents U.S. Pat. Nos. 5,885,613, 6,287,591, 6,320,017, and 6,586,559, the contents of all of which are incorporated herein by reference in their entirety.

In some embodiments, the one or more additional compound can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the ceDNA within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate). In another example, if the LNP containing the ceDNA is useful for treating an infection, the additional compound can be an antimicrobial agent (e.g., an antibiotic or antiviral compound). In yet another example, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a ceDNA encoding a different protein or a different compound, such as a therapeutic may be used in the compositions and methods of the invention.

In some embodiments, the additional compound is an immune modulating agent. For example, the additional compound is an immunosuppressant. In some embodiments, the additional compound is immune stimulatory agent.

Also provided herein is a pharmaceutical composition comprising the lipid nanoparticle-encapsulated ceDNA vector and a pharmaceutically acceptable carrier or excipient. In some embodiments, the pharmaceutical composition further comprise a protein kinase inhibitor (e.g., a TKI) as disclosed herein. In alternative embodiments, a pharmaceutical composition comprising a lipid nanoparticle encapsulated ceDNA vector and a pharmaceutical acceptable carrier or excipient is co-administered to the subject with a pharmaceutical composition comprising a protein kinase inhibitor.

In some aspects, the disclosure provides for a lipid nanoparticle formulation further comprising one or more pharmaceutical excipients. In some embodiments, the lipid nanoparticle formulation further comprises sucrose, tris, trehalose and/or glycine.

A closed-ended DNA vector, including a ceDNA vector, as described herein can be complexed with the lipid portion of the particle or encapsulated in the lipid position of the lipid nanoparticle. In some embodiments, a DNA vector, including a ceDNA vector as described herein can be fully encapsulated in the lipid position of the lipid nanoparticle, thereby protecting it from degradation by a nuclease, e.g., in an aqueous solution. In some embodiments, a DNA vector, including a ceDNA vector as described herein in the lipid nanoparticle is not substantially degraded after exposure of the lipid nanoparticle to a nuclease at 37° C. for at least about 20, 30, 45, or 60 minutes. In some embodiments, the ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human. In some aspects, the lipid nanoparticle formulation is a lyophilized powder.

In some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. For example, the morphology of the lipid nanoparticles (lamellar vs. non-lamellar) can readily be assessed and characterized using, e.g., Cryo-TEM analysis as described in US2010/0130588, the content of which is incorporated herein by reference in its entirety.

In some further embodiments, the lipid nanoparticles having a non-lamellar morphology are electron dense. In some aspects, the disclosure provides for a lipid nanoparticle that is either unilamellar or multilamellar in structure. In some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size.

The pKa of formulated cationic lipids can be correlated with the effectiveness of the LNPs for delivery of nucleic acids (see Jayaraman et al., Angewandte Chemie, International Edition (2012), 51(34), 8529-8533; Semple et al., Nature Biotechnology 28, 172-176 (2010), both of which are incorporated by reference in their entirety). The preferred range of pKa is ˜5 to ˜7. The pKa of the cationic lipid can be determined in lipid nanoparticles using an assay based on fluorescence of 2-(p-toluidino)-6-napthalene sulfonic acid (TNS).

The present invention contemplates the use of anionic (conventional) liposomes, pH sensitive liposomes, immunoliposomes, fusogenic liposomes and cationic lipid/antisense aggregates in conjunction with gene/nucleic acid therapy. Likewise, nucleic acid therapeutics have been administered systemically in the cationic liposomes, and these nucleic-acid containing lipid particles can result in improved downregulation of target proteins in mammals, including non-human primates. Preferably, these compositions can be encapsulated nucleic acids with high efficiency with high drug to lipid ratio, resulting in the encapsulated nucleic acid that is protected from degradation and clearance in serum, while providing a minimal immune responsive state in the subject receiving the treatment, which can be even suitable for a systemic delivery of nucleic acid therapeutics. Furthermore, these approaches involving nucleic acid containing lipid particles as treatment of a patient subject provide safe and efficacious therapy without significant toxicity and/or risk to the patient.

Generally, lipid particles can be formed by any method known in the art. For example, the lipid particles can be prepared by the methods described, for example, in US2013/0037977, US2010/0015218, US2013/0156845, US2013/0164400, US2012/0225129, and US2010/0130588, content of each of which is incorporated herein by reference in its entirety. In some embodiments, lipid particles can be prepared using a continuous mixing method, a direct dilution process, or an in-line dilution process.

### IV. Immunosuppressants

Immunosuppressants described herein include protein kinase inhibitors (PKIs), such as tyrosine kinase inhibitors (TKIs), which include but are not limited to small molecule compounds, biologics (such as monoclonal antibodies), and large polypeptide molecules that inhibit the activity of, for example, IFN signaling and production pathways, or any other form of antagonists that can decrease expression of a target protein in the immune response pathway. It is to be understood that the present invention contemplates use of any modality of therapeutics that can act as an antagonist of, e.g., the IFN signaling and production pathways that modulate immune responses.

The immunosuppressants are protein kinase inhibitors that antagonize the activity of protein kinases, and can be used in conjunction with any nucleic acid therapeutic that triggers an immune response (innate and/or adaptive) in a host cell or a subject suffering from a genetic disorder. Tyrosine kinases regulate a variety of cellular functions including cell growth (e.g., IFN signaling and production and epidermal growth factor (“EGFR” such as ERBB1, ERBB2/HER2, ERBB3/HER3, ERBB4/HER4)). These are the main signal transducers and activators which act downstream of multiple cytokines, growth factors, and hormones, thereby regulating immune responses. For example, upon binding of a specific ligand to its cognate receptor, conformational changes lead to receptor oligomerization and activation of the receptor-associated JAKs. JAKs auto- and trans-phosphorylate one another and phosphorylate receptor chains, providing the docking sites for STAT molecules. STATs then undergo JAK-mediated phosphorylation, dimerize, and translocate to the nucleus, where they regulate the transcription of target genes involving immune responses (e.g., interferon-α, interferon-β, interferon-γ, TNFα, IL2, IL-6, IL-18, etc.).

In some embodiments, the immunosuppressant is an antagonist of Jak1, Jak2, Jak3, Stat, Tyk2, c-MET, EGFR, c-KIT, BTK, ALK, ABL, SRC, ROS1, Syk, MEK, ATM, NRF2, Flt3, fms/CSFIR, FDGFRs, RON, IGF1R, EPHA2, EPHA3, VEGF or VEGFR. In some embodiments, the immunosuppressant is an antagonist of tyrosine kinase. In one embodiment, the immunosuppressant is an antagonist of Jak1. In another embodiment, the immunosuppressant is an antagonist of Jak2. In one embodiment, the immunosuppressant is an antagonist of Jak3. In yet another embodiment, the immunosuppressant is an antagonist of Tyk2. In yet another embodiment, the immunosuppressant is an antagonist of EGFR. In one embodiment, the immunosuppressant is an antagonist of ALK. In yet another embodiment, the immunosuppressant is an antagonist of Syk.

In one embodiment, the immunosuppressant is a small molecule antagonist. In another embodiment, the immunosuppressant is an antibody that binds to a protein kinase target. In another embodiment, the immunosuppressant is an antibody that binds to a tyrosine kinase. In another embodiment, the immunosuppressant is a monoclonal antibody against a protein kinase. In another embodiment, the immunosuppressant is a monoclonal antibody against tyrosine kinase. In another embodiment, the immunosuppressant is a monoclonal antibody against a target selected from the group consisting of Jak1, Jak2, Jak3, Stat, Tyk2, c-MET, EGFR, c-KIT, BTK, ALK, ABL, SRC, ROS1, Syk, MEK, ATM, NRF2, Flt3, fms/CSF1R, FDGFRs, RON, IGF1R, EPHA2, EPHA3, VEGF and VEGFR. In yet another embodiment, the immunosuppressant is a polypeptide that has binding affinity to a protein kinase. In yet another embodiment, the immunosuppressant is a nucleic acid, such as RNAi or an anti-sense oligonucleotide, that attenuates expression of Jak1, Jak2, Jak3, Stat, Tyk2, c-MET, EGFR, c-KIT, BTK, ALK, ABL, SRC, ROS1, Syk, MEK, ATM, NRF2, Flt3, fms/CSF1R, FDGFRs, RON, IGF1R, EPHA2, EPHA3, VEGF or VEGFR.

In some embodiments, inhibition of a protein kinase, e.g., a tyrosine kinase, can be achieved by using small molecules that bind to the ATP pocket of a given protein kinase, blocking it from catalyzing the phosphorylation of target proteins. Hence, in some embodiments, the immunosuppressant can be a small molecule antagonist of protein kinase. Non-limiting examples of immunosuppressant protein kinase antagonist include imatinib mesylate (Gleevec®), Nilotinib (Tasigna®), sorafenib (Nexavar®), sunitinib (Sutent®), dasatinib (Sprycel®), acalabrutinib, alectinib, axitinib, baricitinib, afatinib, bosutinib, brigatinib, cabozantinib, cerdulatinib, ceritinib, cobimetinib, crizotinib, dacomitinib, dasatinib, erlotinib, imatinib, fostamatinib, gefitinib, AG-1478, lapatinib, lorlatinib, TAK-659, ruxolitinib, osimertinib, pazopanib, pegaptanib, ponatinib, regorafenib, saracatinib, tofacitinib, BMS-986165, vandetinib, vemurafenib, or a pharmaceutically acceptable salt thereof.

In some embodiments, the immunosuppressant can be a small molecule antagonist of tyrosine kinase and is selected from the group consisting of baricitinib, afatinib, brigatinib, cerdulatinib, ceritinib, cobimetinib, dacomitinib, dasatinib, osimertinib, fostamatinib, saracatinib, TAK-659, ruxolitinib, BMS-986165, tofacitinib, and a pharmaceutically acceptable salt thereof.

In some embodiments, said TKI is selected from the group consisting of sunitinib, imatinib, sorafenib, dasatinib, entoplestinib, fostamatinib, TAK-659, ruxolitinib, baricitinib, BMS-986165, tofacitinib, and a pharmaceutically acceptable salt thereof.

In some embodiments, the TKI is selected from the group consisting of fostamatinib, ruxolitinib, BMS-986165, and a pharmaceutically acceptable salt thereof.

In one embodiment, the TKI is fostamatinib. In another embodiment, the TKI is ruxolitinib or ruxolitinib phosphate. In yet another embodiment, the TKI is BMS-986165.

In some embodiments, the TKI may selectively inhibit one or multiple kinases; or target multiple kinases in the same pathway. For example, ruxolitinib and baricitinib can inhibit Jak1 and Jak2. Lorlatinib can inhibit ROS1 and ALK. Dasatinib can inhibit Alb, Src and c-Kit. Brigatinib, genfinitinib, erlotinib, AG-1478 and lapatinib can inhibit EGFR. Crizitinib can inhibit both ALK and c-Met. Fostamatinib and cerdulitinib can selectively inhibit Syk. Saracatinib can inhibit Src and Abl. In some embodiments, the TKI is an inhibitor of Jak1. In some embodiments, the TKI is an inhibitor of Jak2. In some embodiments, the TKI is an inhibitor of Jak1 and Jak2. In some embodiments, the TKI is an inhibitor of EGFR. In some embodiments, the TKI is an inhibitor of ALK. In some embodiments, the TKI is an inhibitor of Syk.

Protein kinase activity in immune response pathways may also be inhibited by biologic drugs, such as a monoclonal antibody against a protein kinase. These therapeutics may exert efficacy by preventing receptor protein kinases from activating and are capable of binding cell surface antigens with high specificity. Several monoclonal antibodies target receptor protein kinases that play a role in inhibiting protein kinases involving in DNA sensing immune response signaling pathways. Trastuzumab and bevacizumab are nonlimiting examples of such monoclonal antibodies.

In some embodiments, the biologic agent that functions to suppress unwanted immune response to a TNA is a monoclonal antibody selected from the group consisting of ado-trastuzumab emtansine, cetuximab, CetuGEX™, cixutumumab, dalotuzumab, duligotumab, ertumaxomab, futuximab, ganitumab, icrucumab, margetuximab, namatumab, necitumumab, nimotuzumab (h-R3), olaratumab, onartuzumab, panitumumab, pertuzumab, ranibizumab, ramucirumab, seribantumab, tanibirumab, teprotumumab, trasGEX™, trastuzumab, and zatuximab. In one embodiment, the monoclonal antibody is trastuzumab. In another embodiment, the monoclonal antibody is cetuximab.

In some embodiments, the protein kinase inhibitor is a peptide. The peptide can be polypeptide having a specific affinity binding to target proteins such as Jak1, Jak2, Jak3, STAT, Tyk2, c-MET, EGFR, c-KIT, BTK, ALK, ABL, SRC, ROS1, Syk, MEK, ATM, NRF2, Flt3, fms/CSF1R, FDGFRs, RON, IGF1r, EPHA2, EPHA3, VEGF or VEGFR. Non-limiting examples of a polypeptide immunosuppressant include aflibercept (VEGF). Binding targets for the peptide can be in a signaling pathway involved in, e.g., IFN response and production pathways.

In some embodiments, the immunosuppressants of the present invention effectively reduce in vitro and in vivo pro-inflammatory cytokine and chemokine levels when the immunosuppressants are present in combination with the nucleic acid therapeutics. The pro-inflammatory cytokine can be selected from any or a combination of interferon-α (IFN-α), interferon-γ (IFN-γ), tumor necrosis factor-α (TNF-α), interleukin-10 (IL-1p), interleukin-6 (IL-6), interleukin-8 (IL-8), interleukin-10 (IL-10), interleukin-12 (IL-12), interleukin-18 (IL-18), vascular endothelial growth factor (VEGF), leukemia inhibitory factor (LIF), matrixmetalloproteinase 2 (MMP2), monocyte chemoattractant protein-1 (MCP-1), RANTES (CCL5), IP-10 (CXCL10), macrophage inflammatory protein-1α (MIP-1α; CCL3) and/or macrophage inflammatory protein-1β (MIP-1β; CCL4).

### V. Pharmaceutical Compositions and Formulations

The present invention contemplates pharmaceutical compositions comprising a therapeutic nucleic acid and one or more immunosuppressants described herein. In some embodiments, the pharmaceutical composition comprising a therapeutic nucleic acid and one or more immunosuppressants may include a pharmaceutically acceptable excipient or carrier.

In some embodiments, a pharmaceutical composition comprising a therapeutic nucleic acid and immunosuppressant of the present invention may comprise a lipid particle as a carrier. Such a lipid formulation can be used to deliver an immunomodulatory active agent (immunosuppressants, such as TKIs) and/or a nucleic acid therapeutics, to a target site of interest (e.g., cell, tissue, organ, and the like). In preferred embodiments, lipid particles can be a therapeutic nucleic acid containing lipid particle, which is typically formed from a cationic lipid, a non-cationic lipid, and optionally a conjugated lipid that prevents aggregation of the particles.

In some embodiments, the lipid particles are provided with full encapsulation, partial encapsulation of the therapeutic nucleic acid and/or an immunosuppressant. In a preferred embodiment, the nucleic acid therapeutics is fully encapsulated in the lipid particles to form a nucleic acid containing lipid particle that can optionally include any of the immunosuppressants of the present invention if the nucleic acid therapeutics and the immunosuppressant are to be co-formulated and administered to a subject simultaneously. In some embodiments, the immunosuppressants and/or the nucleic acid may be encapsulated within the lipid portion of the particle, thereby protecting it from enzymatic degradation. In some other embodiments, the lipid particles comprising a therapeutic nucleic acid and/or an immunosuppressant typically have a mean diameter of from about 20 nm to about 100 nm, 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm to ensure effective delivery. Nucleic acid containing lipid particles and their method of preparation are disclosed in, e.g., PCT/US18/50042, U.S. Patent Publication Nos. 20040142025 and 20070042031, the disclosures of which are herein incorporated by reference in their entirety for all purposes.

In some embodiments, the lipid particles may be conjugated with other moieties to prevent aggregation. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

In some embodiments, a liquid pharmaceutical composition comprising a therapeutic nucleic acid and/or immunosuppressant of the present invention may be formulated in lipid particles. In some embodiments, the lipid particle comprising a therapeutic nucleic acid can be formed from a cationic lipid. In some other embodiments, the lipid particle comprising a therapeutic nucleic acid can be formed from non-cationic lipid. In a preferred embodiment, the lipid particle of the invention is a nucleic acid containing lipid particle, which is formed from a cationic lipid comprising a therapeutic nucleic acid selected from the group consisting of mRNA, antisense RNA and oligonucleotide, ribozymes, aptamer, interfering RNAs (RNAi), Dicer-substrate dsRNA, small hairpin RNA (shRNA), asymmetrical interfering RNA (aiRNA), microRNA (miRNA), minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone (dbDNA™) DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

In another preferred embodiment, the lipid particle of the invention is a nucleic acid containing lipid particle, which is formed from a non-cationic lipid, and optionally a conjugated lipid that prevents aggregation of the particle. In some other embodiments, the lipid particle of the invention is a nucleic acid containing lipid particle comprising one or more immunosuppressants of the present disclosure, e.g., imatinib mesylate (Gleevec®), Nilotinib (Tasigna®), sorafenib (Nexavar®), sunitinib (Sutent®), dasatinib (Sprycel®), acalabrutinib, alectinib, axitinib, baricitinib, afatinib, bosutinib, brigatinib, cabozantinib, cerdulatinib, ceritinib, cobimetinib, crizotinib, dacomitinib, dasatinib, erlotinib, imatinib, fostamatinib, gefitinib, AG-1478, lapatinib, lorlatinib, TAK-659, ruxolitinib, osimertinib, pazopanib, pegaptanib, ponatinib, regorafenib, saracatinib, tofacitinib, BMS-986165, vandetinib, vemurafenib, or a pharmaceutically acceptable salt thereof.

### Unit Dosage

According to some embodiments, the pharmaceutical compositions can be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### VI. Administration and Dosing

The disclosure provided herein describes methods to prevent, reduce or eliminate unwanted immune response in a subject (e.g., a human subject) by administering to the subject at least one immunosuppressant (or derivative or salt thereof) and a nucleic acid therapeutic, wherein the administrations of the immunosuppressant and the administration of the nucleic acid therapeutic are correlated in time so as to provide a modulation in an immune response when the administration of the two agents are provided in combination. Methods disclosed herein can comprise administering to the subject a combination of an immunosuppressant (e.g., TKI or derivative or salt thereof) and a therapeutic nucleic acid in an effective amount to ameliorate a genetic disorder with a sufficient level of reduction in immune responses which allows for the safe administration of the therapeutic nucleic acid. These two agents can be administered at the same time in a co-formulation, at the same time in different formulations, or they can be administered separately at different times.

The immunosuppressant(s) and TNA(s) may be administered to the subject or patient in any combination. In some embodiments, only one immunosuppressant, e.g., ruxolitinib or a derivative or salt thereof (ruxolitinib phosphate), is administered to a subject or patient. In a particular example, a subject or patient described herein may be administered a therapeutically effective dose of TKI (or derivative or salt thereof). In some cases, two immunosuppressants (or derivative or salt thereof) are administered to a subject. The two immunosuppressants are selected from the group consisting of imatinib mesylate (Gleevec®), Nilotinib (Tasigna®), sorafenib (Nexavar®), sunitinib (Sutent®), dasatinib (Sprycel®), acalabrutinib, alectinib, baricitinib, afatinib, brigatinib, crizotinib, dacomitinib, dasatinib, lorlatinib, osimertinib, fostamatinib, saracatinib, AG-1478, cobimetinib, ceritinib, lapatinib, gefitinib, erlotinib, ruxolitinib, cerdulatinib, tofacitinib, TAK-659, BMS-986165, vandetinib, and bosutinib. When two or more immunosuppressants are used, they may be administered simultaneously or sequentially in certain order. In some cases, the two or more immunosuppressants may be administered sequentially in particular order. For example, the subject may first be administered ruxolitinib and subsequently administered BMS-986165, both prior to the administration of therapeutic nucleic acid. Alternatively, the subject may first be given ruxolitinib, followed by administration of a therapeutic nucleic acid second, followed by administration of BMS-986165 third.

In some embodiments, a subject may be administered one or more immunosuppressants (or derivative or salt thereof) and one or more nucleic acid therapeutics concomitantly. For example, the method may comprise administering to a subject an immunosuppressant and a nucleic acid therapeutic as two separate formulations but concomitantly. In another example, the method may comprise simultaneously administering to a subject an immunosuppressant and a therapeutic nucleic acid in one formulation, thereby the immunosuppressant and the therapeutic nucleic acid can be administered to a subject at the same time.

In some embodiment, a subject may be administered one or more immunosuppressants (or derivative or salt thereof) and one or more therapeutic nucleic acid sequentially. For example, the immunosuppressant may be administered prior to administration of a therapeutic nucleic acid. For example, a subject may be administered a therapeutically effective dose of an immunosuppressant, and subsequently administered a therapeutic nucleic acid (e.g., minicircle, minigene, ministring covalently closed DNA, doggybone (dbDNA™) DNA, dumbbell shaped DNA, linear closed-ended duplex DNA (ceDNA and CELiD), plasmid based circular vector, antisense oligonucleotide (ASO), RNAi, siRNA, mRNA, etc.). In some cases, the subject is administered with one or more immunosuppressants after receiving a TNA. In some cases, the subject is administered with one or more immunosuppressants and a therapeutic nucleic acid at the same time.

In cases of sequential administration, there may be a delay period between administration of the one or more immunosuppressant and TNAs. For example, the immunosuppressant may be administered hours, days, or weeks prior to administration of the TNA (e.g., at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least about 2 days, at least about 3 days, at least about 4 days, at least about 5 days, at least about 6 days, at least about 1 week, at least about 2 weeks, at least about 3 weeks, and at least about 4 weeks prior to the administration of a therapeutic nucleic acid). In some embodiments, an immunosuppressant may be administered about thirty (30) minutes prior to the administration of a TNA. In some embodiments, an immunosuppressant may be administered about one (1) hour prior to the administration of a therapeutic nucleic acid. In some embodiments, an immunosuppressant can be administered about two (2) hours prior to the administration of a therapeutic nucleic acid. In some embodiments, an immunosuppressant can be administered about three (3) hours prior to the administration of a therapeutic nucleic acid. In some embodiments, an immunosuppressant can be administered about four (4) hours prior to the administration of a therapeutic nucleic acid. In some embodiments, an immunosuppressant can be administered about five (5) hours prior to the administration of a therapeutic nucleic acid. In some embodiments, an immunosuppressant can be administered about six (6) hours prior to the administration of a therapeutic nucleic acid. In some embodiments, an immunosuppressant can be administered about seven (7) hours prior to the administration of a therapeutic nucleic acid. In some embodiments, an immunosuppressant can be administered about eight (8) hours prior to the administration of a therapeutic nucleic acid. In some embodiments, an immunosuppressant can be administered about nine (9) hours prior to the administration of a therapeutic nucleic acid. In some embodiments, an immunosuppressant can be administered about ten (10) hours prior to the administration of a therapeutic nucleic acid.

In one embodiment, an immunosuppressant is administered no more than about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or 24 hours before the administration of a therapeutic nucleic acid. In some embodiments, an immunosuppressant can be administered no more than about 1 day, about 2 days, about 3 days, about 4 days, about 6 days, or about 7 days before the administration of a therapeutic nucleic acid.

In some embodiments, an immunosuppressant can be administered about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or 24 hours after the administration of a therapeutic nucleic acid. In some embodiments, an immunosuppressant can be administered about 1 day, about 2 days, about 3 days, about 4 days, about 6 days, or about 7 days after the administration of a therapeutic nucleic acid.

In one embodiment, an immunosuppressant is administered no more than about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or 24 hours after the administration of a therapeutic nucleic acid. In some embodiments, an immunosuppressant can be administered no more than about 1 day, about 2 days, about 3 days, about 4 days, about 6 days, or about 7 days after the administration of a therapeutic nucleic acid.

In some embodiments, one or more immunosuppressants can be administered multiple times before, concurrently with, and/or after the administration of a therapeutic nucleic acid.

In some embodiments, a therapeutic nucleic acid (e.g., a ceDNA vector) can be administered as a single dose or as multiple doses. According to some embodiments, more than one dose can be administered to a subject. Multiple doses can be administered as needed, because the ceDNA vector does not elicit an anti-capsid host immune response due to the absence of a viral capsid. According to some embodiments the number of doses administered can, for example, be between 2-10 or more doses, for example 2, 3, 4, 5, 6, 7, 8, 9, 10 or more.

In some embodiments, a therapeutic nucleic acid can be administered and re-dosed multiple times in conjunction with one or more immunosuppressant disclosed herein. For example, the therapeutic nucleic acid can be administered on day 0 with one or more immunosuppressants that is administered before, after or at the same time with the administration the therapeutic nucleic acid in a first dosing regimen. Following the initial treatment at day 0, a second dosing (re-dose) can be performed in about 1 week, about 2 weeks, about 3 weeks, about 4 weeks, about 5 weeks, about 6 weeks, about 7 weeks, about 8 weeks, or about 3 months, about 4 months, about 5 months, about 6 months, about 7 months, about 8 months, about 9 months, about 10 months, about 11 months, or about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, about 31 years, about 32 years, about 33 years, about 34 years, about 35 years, about 36 years, about 37 years, about 38 years, about 39 years, about 40 years, about 41 years, about 42 years, about 43 years, about 44 years, about 45 years, about 46 years, about 47 years, about 48 years, about 49 years or about 50 years after the initial treatment with the therapeutic nucleic acid, preferably with one or more immunosuppressants disclosed herein.

According to some embodiments, re-dosing of the therapeutic nucleic acid results in an increase in expression of the therapeutic nucleic acid. According to some embodiments, the increase of expression of the therapeutic nucleic acid after re-dosing, compared to the expression of the therapeutic nucleic acid after the first dose is about 0.5-fold to about 10-fold, about 1-fold to about 5-fold, about 1-fold to about 2-fold, or about 0.5-fold, about 1-fold, about 2-fold, about 3-fold, about 4-fold, about 5-fold, about 6-fold, about 7-fold, about 8-fold, about 9-fold or about 10-fold higher after re-dosing of the therapeutic nucleic acid.

According to some embodiments, more than one administration (e.g., two, three, four or more administrations) of a therapeutic nucleic acid (e.g., a ceDNA vector) for expression of a therapeutic protein as disclosed herein may be employed to achieve a desired level of gene expression over a period of various intervals, e.g., daily, weekly, monthly, yearly, etc.

The immunosuppressants of the current disclosure may be administered by any of the accepted modes of administration, for example, by cutaneous, oral, topical, intradermal, intrathecal, intravenous, subcutaneous, intramuscular, intratumoral, intra-articular, intraspinal or spinal, nasal, epidural, rectal, vaginal, or transdermal/transmucosal routes. The most suitable route will depend on the nature and severity of the disorder and condition of the subject. Subcutaneous, oral, intradermal, intravenous and percutaneous administration can be routes for the immunosuppressants of this disclosure. Sublingual administration may be a route of administration for the immunosuppressants of this disclosure. Intravenous administration may be a route of administration for the immunosuppressants of this disclosure. In one particular example, the immunosuppressants provided herein may be administered to a subject orally.

In some embodiments, the immunosuppressant and the nucleic acid therapeutics are each formulated in a solution. In some embodiments, one or more immunosuppressant and a therapeutic nucleic acid can be co-formulation in a liquid solution. Such a pharmaceutical composition in a liquid solution can be pharmaceutically acceptable excipient or carrier, e.g., for oral delivery, injection, infusion, subcutaneous delivery, intramuscular delivery, intraperitoneal delivery, intrathecal delivery, intratumoral delivery, sublingual delivery, or other method described herein. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. In another embodiment, for treatment of an ophthalmological condition or disease, a liquid pharmaceutical composition may be applied to the eye in the form of eye drops. A liquid pharmaceutical composition may be delivered orally as well.

### Dosing

Immunosuppressants

The dosing regimen of the immunosuppressants (e.g., protein kinase immunosuppressants) according to the present disclosure may vary depending upon the indication, route of administration, and severity of the condition, for example, depending on the route of administration, a suitable dose can be calculated according to body weight, body surface area, or organ size. The final dosing regimen is determined by the attending physician in view of good medical practice, considering various factors that modify the action of drugs, e.g., the specific activity of the compound, the identity and severity of the disease state, the responsiveness of the patient, the age, condition, body weight, sex, and diet of the patient, and the severity of any infection. Additional factors that can be taken into account include time and frequency of administration, drug combinations, reaction sensitivities, and tolerance/response to therapy. Further refinement of the doses appropriate for treatment involving any of the formulations mentioned herein is done routinely by the skilled physician or practitioner without undue experimentation. Appropriate doses can be ascertained through use of established assays for determining concentration of the agent in a body fluid or other sample together with dose response data.

According to some embodiments, the immunosuppressant is administered in a therapeutically effective amount. Determination of a therapeutically effective amount is within the capability of those skilled in the art, and in view of the disclosure provided herein.

According to some embodiments the immunosuppressant is administered at a dose of between about 0.5 mg/kg bodyweight to about 700 mg/kg bodyweight, or between about 0.5 mg/kg bodyweight to about 500 mg/kg bodyweight, or between about 0.5 mg/kg bodyweight to about 250 mg/kg bodyweight, or between about 0.5 mg/kg bodyweight to about 150 mg/kg bodyweight, or between about 0.5 mg/kg bodyweight to about 100 mg/kg bodyweight, or between about 0.5 mg/kg bodyweight to about 50 mg/kg bodyweight, or between about 0.5 mg/kg bodyweight to about 10 mg/kg bodyweight, or between about 10 mg/kg bodyweight to about 700 mg/kg bodyweight, or between about 10 mg/kg bodyweight to about 500 mg/kg bodyweight, or between about 10 mg/kg bodyweight to about 250 mg/kg bodyweight, or between about 10 mg/kg bodyweight to about 100 mg/kg bodyweight, or between about 10 mg/kg bodyweight to about 50 mg/kg bodyweight, or between about 50 mg/kg bodyweight to about 500 mg/kg bodyweight, or between about 50 mg/kg bodyweight to about 250 mg/kg bodyweight, or between about 50 mg/kg bodyweight to about 150 mg/kg bodyweight, or between about 50 mg/kg bodyweight to about 100 mg/kg bodyweight, or between about 100 mg/kg bodyweight to about 700 mg/kg bodyweight, or between about 100 mg/kg bodyweight to about 500 mg/kg bodyweight, or between about 100 mg/kg bodyweight to about 250 mg/kg bodyweight, or between about 100 mg/kg bodyweight to about 150 mg/kg bodyweight, or between about 200 mg/kg bodyweight to about 700 mg/kg bodyweight, or between about 200 mg/kg bodyweight to about 500 mg/kg bodyweight, or between about 200 mg/kg bodyweight to about 250 mg/kg bodyweight, or between about 300 mg/kg bodyweight to about 700 mg/kg bodyweight, or between about 300 mg/kg bodyweight to about 600 mg/kg bodyweight, or between about 300 mg/kg bodyweight to about 500 mg/kg bodyweight, or between about 300 mg/kg bodyweight to about 400 mg/kg bodyweight, or between about 400 mg/kg bodyweight to about 700 mg/kg bodyweight, or between about 400 mg/kg bodyweight to about 600 mg/kg bodyweight, or between about 400 mg/kg bodyweight to about 500 mg/kg bodyweight, or between about 400 mg/kg bodyweight to about 450 mg/kg bodyweight, or between about 5300 mg/kg bodyweight to about 700 mg/kg bodyweight, or between about 500 mg/kg bodyweight to about 650 mg/kg bodyweight, or between about 500 mg/kg bodyweight to about 600 mg/kg bodyweight, or between about 600 mg/kg bodyweight to about 700 mg/kg bodyweight, or between about 650 mg/kg bodyweight to about 700 mg/kg bodyweight, or about 0.5 mg/kg, or about 1 mg/kg, or about 5 mg/kg, or about 10 mg/kg, or about 15 mg/kg, or about 20 mg/kg, or about 25 mg/kg, or about 30 mg/kg, or about 35 mg/kg, or about 40 mg/kg, or about 45 mg/kg, or about 50 mg/kg, or about 75 mg/kg, or about 100 mg/kg, or about 125 mg/kg, or about 150 mg/kg, or about 175 mg/kg, or about 200 mg/kg, or about 225 mg/kg, or about 250 mg/kg, or about 275 mg/kg, or about 300 mg/kg, or about 325 mg/kg, or about 350 mg/kg, or about 375 mg/kg, or about 400 mg/kg, or about 425 mg/kg, or about 450 mg/kg, or about 475 mg/kg, or about 500 mg/kg, or about 525 mg/kg, or about 550 mg/kg, or about 575 mg/kg, or about 600 mg/kg, or about 625 mg/kg, or about 650 mg/kg, or about 675 mg/kg, or about 700 mg/kg bodyweight.

Therapeutic Nucleic Acids

According to some embodiments, a therapeutic nucleic acid as described herein can be administered to an organism for transduction of cells in vivo or ex vivo.

Generally, administration of the therapeutic nucleic acid is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Exemplary modes of administration of the include oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain). According to some embodiments, the therapeutic nucleic acid is administered intravenously.

Administration of the therapeutic nucleic acid can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. In one embodiment, administration of the therapeutic nucleic acid can also be to a tumor (e.g., in or near a tumor or a lymph node). The most suitable route in any given case will depend on the nature and severity of the condition being treated, ameliorated, and/or prevented and on the nature of the particular therapeutic nucleic acid that is being used.

The dose of the amount of therapeutic nucleic acid for expression of a therapeutic protein as disclosed herein required to achieve a particular“therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a dose range of therapeutic nucleic acid to treat a patient having a particular disease or disorder based on the aforementioned factors, as well as other factors that are well known in the art.

Depending on the type and severity of the disease, a therapeutic nucleic acid is administered in an amount that the encoded therapeutic protein is expressed at about 0.3 mg/kg to about 100 mg/kg, for example about 0.5 mg/kg to about 100 mg/kg, about 1 mg/kg to about 100 mg/kg, about 5 mg/kg to about 100 mg/kg, about 10 mg/kg to about 100 mg/kg, about 20 mg/kg to about 100 mg/kg, about 30 mg/kg to about 100 mg/kg, about 40 mg/kg to about 100 mg/kg, about 50 mg/kg to about 100 mg/kg, about 60 mg/kg to about 100 mg/kg, about 70 mg/kg to about 100 mg/kg, about 80 mg/kg to about 100 mg/kg, about 90 mg/kg to about 100 mg/kg, by one or more separate administrations, or by continuous infusion. According to some embodiments, the therapeutic nucleic acid is administered in an amount sufficient to result in the expression of the encoded therapeutic protein for a total dose in the range of 50 mg to 2500 mg. An exemplary dose of a therapeutic nucleic acid is an amount sufficient to result in the total expression of the encoded therapeutic protein at about 50 mg, about 100 mg, 200 mg, 300 mg, 400 mg, about 500 mg, about 600 mg, about 700 mg, about 720 mg, about 1000 mg, about 1050 mg, about 1100 mg, about 1200 mg, about 1300 mg, about 1400 mg, about 1500 mg, about 1600 mg, about 1700 mg, about 1800 mg, about 1900 mg, about 2000 mg, about 2050 mg, about 2100 mg, about 2200 mg, about 2300 mg, about 2400 mg, or about 2500 mg (or any combination thereof). As the expression of the therapeutic protein. According to some embodiments, a therapeutic nucleic acid is administered an amount sufficient to result in the expression of the encoded therapeutic protein at a dose of 15 mg/kg, 30 mg/kg, 40 mg/kg, 45 mg/kg, 50 mg/kg, 60 mg/kg, 70 mg/kg, 80 mg/kg, 90 mg/kg, 100 mg/kg or more.

The efficacy of a ceDNA vector as described herein administered with a protein kinase inhibitor as described herein, for suppressing or reducing the innate immune system, can be determined by the skilled clinician. According to some embodiments, a treatment is considered “effective treatment,” as the term is used herein, if any one or all of the signs or symptoms of the innate immune system are reduced and/or are altered in a beneficial manner, or other clinically accepted symptoms or markers of disease are improved, or ameliorated, e.g., by at least 10% after treatment with a pharmaceutical composition as disclosed herein. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease or disorder; or (2) relieving the disease, e.g., causing regression of symptoms; and (3) preventing or reducing the likelihood of the development of the disease, or preventing secondary diseases/disorders associated with the disease, such as liver or kidney failure. An effective amount for the treatment of a disease means that amount which, when administered to a mammal in need thereof, is sufficient to result in effective treatment as that term is defined herein, for that disease.

### EXAMPLES

### Example 1. Identification of Inhibitors of ceDNA-Mediated Immune Response and In Vitro Evaluation of Immunomodulators

Protein kinase inhibitors were selected and tested for their activity as immunosuppressant against nucleic acid therapeutics such as ceDNA. Initially, acalabrutinib, alectinib, baricitinib, afatinib, brigatinib, crizotinib, dacomitinib, dasatinib, lorlatinib, osimertinib, fostamatinib, saracatinib, AG-1478, cobimetinib, ceritinib, lapatinib, gefitinib, erlotinib, ruxolitinib, cerdulatinib, tofacitinib, vandetinib, and bosutinib were selected. Relevant structures and kinase targets for these compounds are shown in Table 1 below.

TABLE 1

| | Compound | | |
|---|---|---|---|
| No. | Name | Target | Structure |
| 1 | Acalabrutinib | Bruton's tyrosine kinase (BTK) | 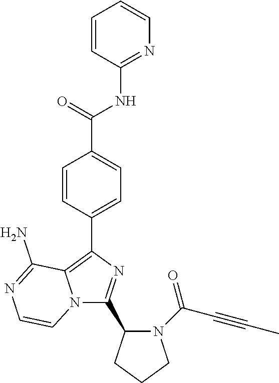 [SMILES: CC#CC(=O)N1CCC[C@H]1c1nc(-c2ccc(C(=O)Nc3ccccn3)cc2)c2c(N)nccn12] |
| 2 | Alectinib/ Alectinib hydrochloride | ALK | 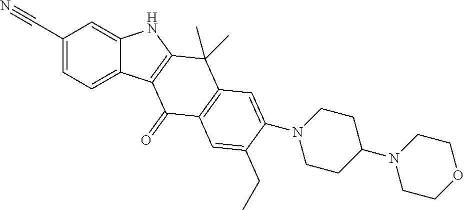 [SMILES: CCc1cc2c(cc1N1CCC(N3CCOCC3)CC1)C(C)(C)c1[nH]c3cc(C#N)ccc3c1C2=O] |
| 3 | Baricitinib | Jak1/2 | 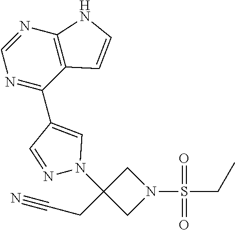 [SMILES: CCS(=O)(=O)N1CC(CC#N)(n2cc(-c3ncnc4[nH]ccc34)cn2)C1] |
| 4 | Afatinib/ Afatinib Dimaleate | EGFR/HER2 | 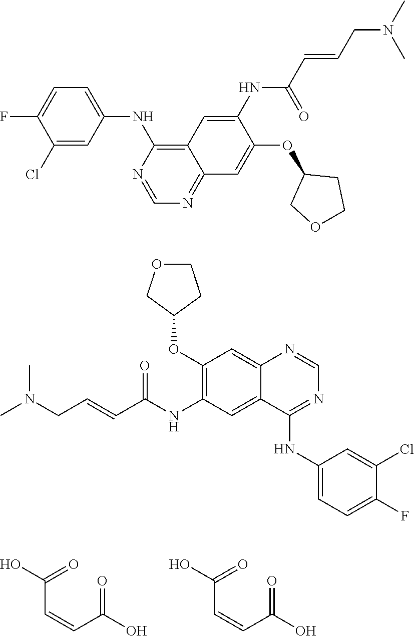 |
| 5 | Brigatinib | EGFR | 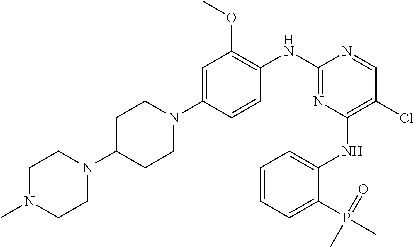 [SMILES: COc1cc(N2CCC(N3CCN(C)CC3)CC2)ccc1Nc1ncc(Cl)c(Nc2ccccc2P(C)(C)=O)n1] |
| 6 | Crizotinib | ALK/c-Met | 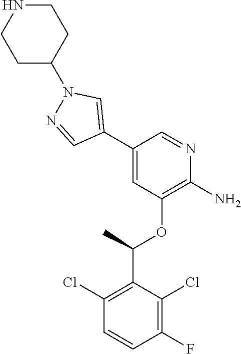 [SMILES: C[C@@H](Oc1cc(-c2cnn(C3CCNCC3)c2)cnc1N)c1c(Cl)ccc(F)c1Cl] |
| 7 | Dacomitinib | EGFR family/pan ErbB | 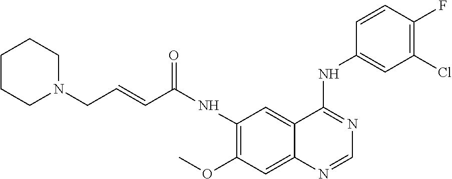 [SMILES: COc1cc2ncnc(Nc3ccc(F)c(Cl)c3)c2cc1NC(=O)/C=C/CN1CCCCC1] |
| 8 | Dasatinib | ABL/Src/c-Kit | 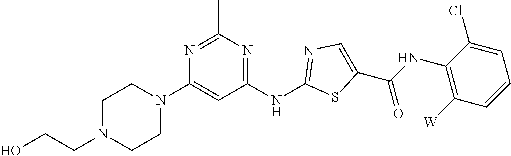 |
| 9 | Lorlatinib | ROS1/ALK | 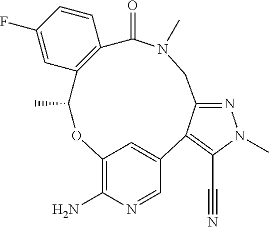 [SMILES: C[C@H]1Oc2cc(cnc2N)-c2c(nn(C)c2C#N)CN(C)C(=O)c2ccc(F)cc21] |
| 10 | Osimertinib/ Osimertinib mesylate | EGFR | 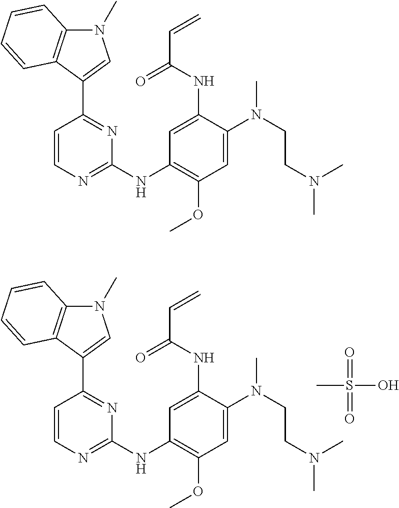 |
| 11 | Fostamatinib/ Fostamatinib disodium | Syk | 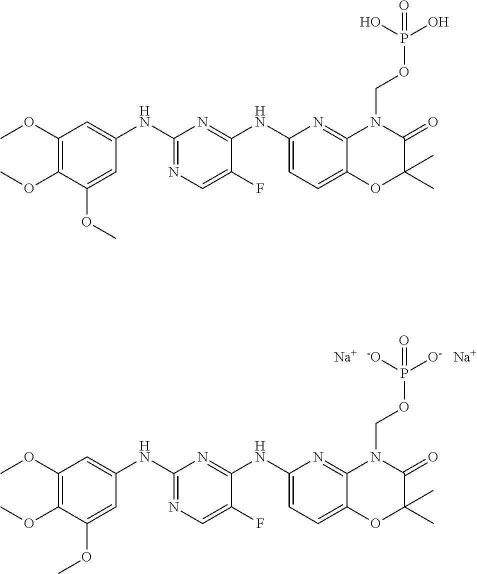 |
| 12 | Saracatinib | Src/Abl | 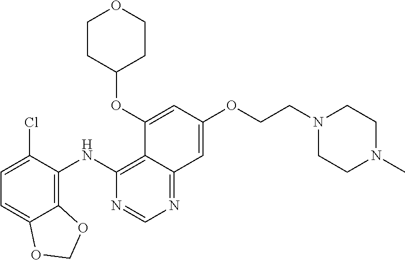 [SMILES: CN1CCN(CCOc2cc(OC3CCOCC3)c3c(Nc4c(Cl)ccc5c4OCO5)ncnc3c2)CC1] |
| 13 | AG-1478 (Tyrphostin AG- 1478) | EGFR | 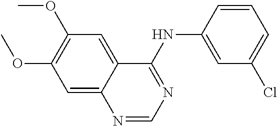 [SMILES: COc1cc2ncnc(Nc3cccc(Cl)c3)c2cc1OC] |
| 14 | Cobimetinib | MEK | 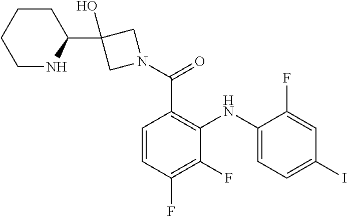 [SMILES: O=C(c1ccc(F)c(F)c1Nc1ccc(I)cc1F)N1CC(O)([C@@H]2CCCCN2)C1] |
| 15 | Ceritinib | ALK | 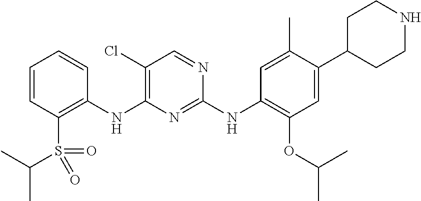 [SMILES: Cc1cc(Nc2ncc(Cl)c(Nc3ccccc3S(=O)(=O)C(C)C)n2)c(OC(C)C)cc1C1CCNCC1] |
| 16 | Lapatinib | EGFR | 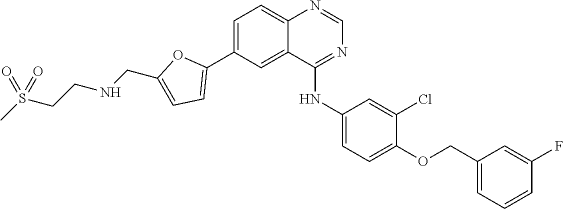 [SMILES: CS(=O)(=O)CCNCc1ccc(-c2ccc3ncnc(Nc4ccc(OCc5cccc(F)c5)c(Cl)c4)c3c2)o1] |
| 17 | Gefitinib/ Gefitinib HCl | EGFR | 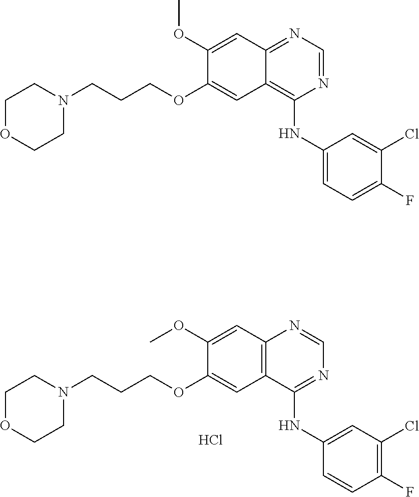 [SMILES: COc1cc2ncnc(Nc3ccc(F)c(Cl)c3)c2cc1OCCCN1CCOCC1 ; COc1cc2ncnc(Nc3ccc(F)c(Cl)c3)c2cc1OCCCN1CCOCC1 ; Cl] |
| 18 | Erlotinib/ Erlotinib HCl | EGFR | 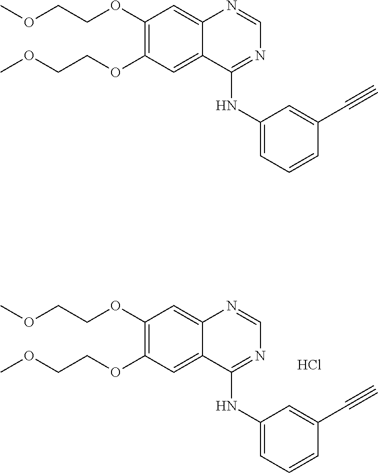 |
| 19 | Ruxolitinib/ Ruxolitinib phosphate/ S-ruxolitinib | Jak1/2 | 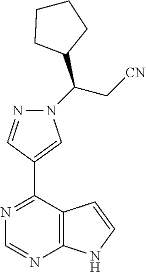 [SMILES: N#CC[C@H](C1CCCC1)n1cc(-c2ncnc3[nH]ccc23)cn1] |
| | | | 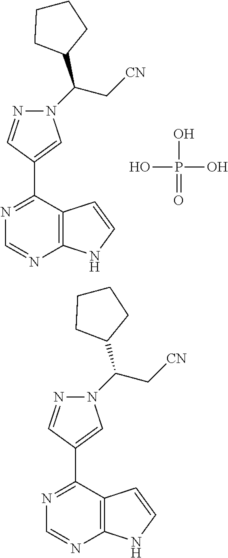 |
| 20 | Cerdulitinib | Syk | 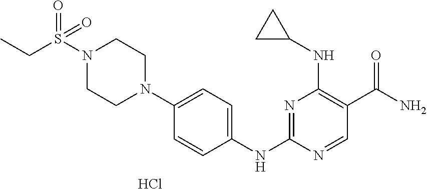 [SMILES: CCS(=O)(=O)N1CCN(c2ccc(Nc3ncc(C(N)=O)c(NC4CC4)n3)cc2)CC1 ; Cl] |
| 21 | Tofacitinib | Jak3 | 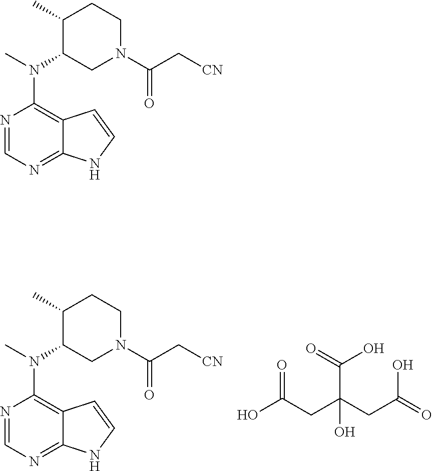 [SMILES: C[C@@H]1CCN(C(=O)CC#N)C[C@@H]1N(C)c1ncnc2[nH]ccc12 ; C[C@@H]1CCN(C(=O)CC#N)C[C@@H]1N(C)c1ncnc2[nH]ccc12 ; O=C(O)CC(O)(CC(=O)O)C(=O)O] |
| 22 | Vandetinib | EGFR | 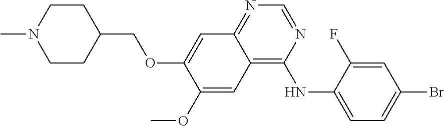 [SMILES: COc1cc2c(Nc3ccc(Br)cc3F)ncnc2cc1OCC1CCN(C)CC1] |
| 23 | Bosutinib | Abl/Src | 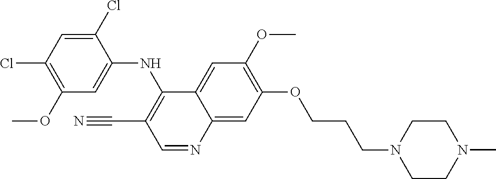 [SMILES: COc1cc(Nc2c(C#N)cnc3cc(OCCCN4CCN(C)CC4)c(OC)cc23)c(Cl)cc1Cl] |
| 24 | BMS986165 Tyk2-IN-4 | Tyk2 | 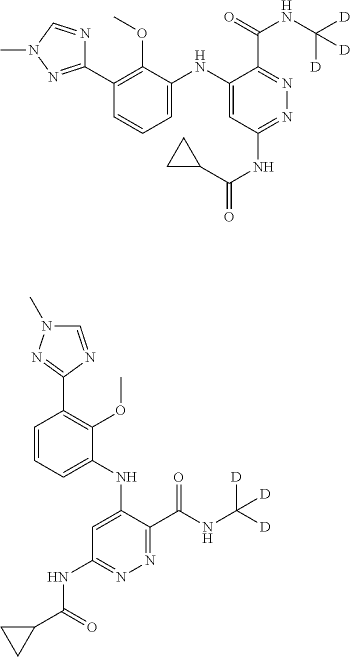 [SMILES: [2H]C([2H])([2H])NC(=O)c1nnc(NC(=O)C2CC2)cc1Nc1cccc(-c2ncn(C)n2)c1OC ; [2H]C([2H])([2H])NC(=O)c1nnc(NC(=O)C2CC2)cc1Nc1cccc(-c2ncn(C)n2)c1OC] |
| 25 | Sunitinib | PDGFRs/ VEGFRs | 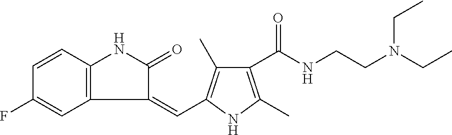 [SMILES: CCN(CC)CCNC(=O)c1c(C)[nH]c(/C=C2\C(=O)Nc3ccc(F)cc32)c1C] |
| 26 | Imatinib/ Imatinib mesylate | Abl/c-kit/ PDGF-R | 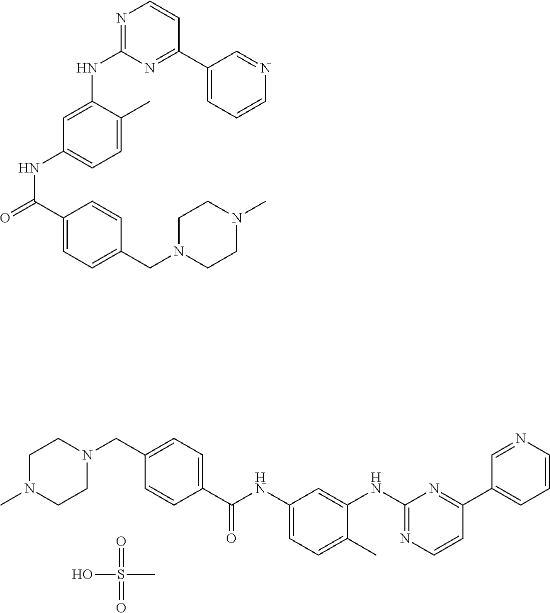 |
| 27 | Sorafenib | VEGFR/ PDGFR/ RAF kinase | 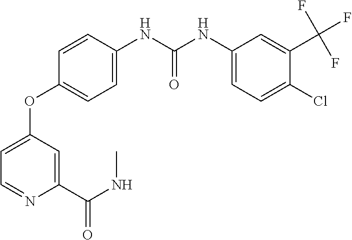 [SMILES: CNC(=O)c1cc(Oc2ccc(NC(=O)Nc3ccc(Cl)c(C(F)(F)F)c3)cc2)ccn1] |
| 28 | Entosplestinib | Syk | 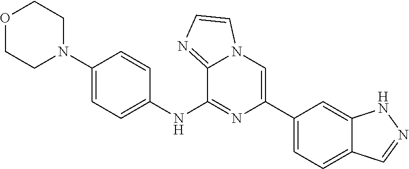 [SMILES: c1cn2cc(-c3ccc4cn[nH]c4c3)nc(Nc3ccc(N4CCOCC4)cc3)c2n1] |
| 29 | TAK-659 | Syk | 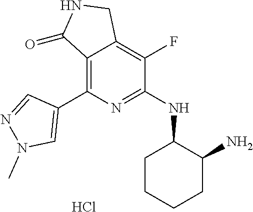 [SMILES: Cn1cc(-c2nc(N[C@@H]3CCCC[C@@H]3N)c(F)c3c2C(=O)NC3)cn1 ; Cl] |

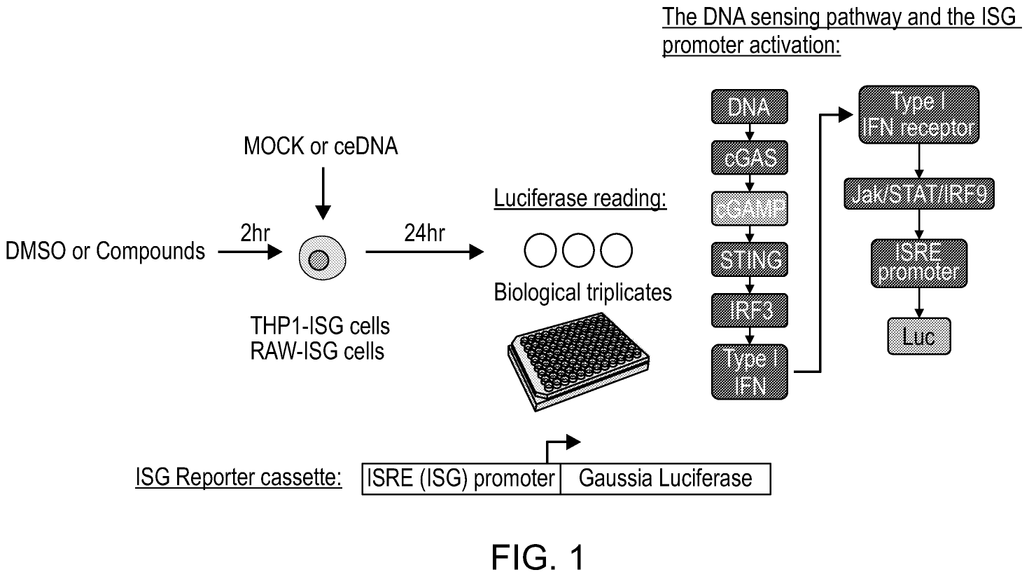

THP1-ISG (interferon-stimulated genes) cells were used to monitor the interferon (IFN) signaling pathway in a physiologically relevant cell line. THP1 cells are derived from the human monocyte cell line by stable integration of an IFN regulatory factor (IRF)-inducible SEAP reporter construct. THP1-ISG cells express a “secreted form of embryonic alkaline phosphatase” (SEAP) reporter gene under the control of an ISG minimal promoter (FIG. 1).

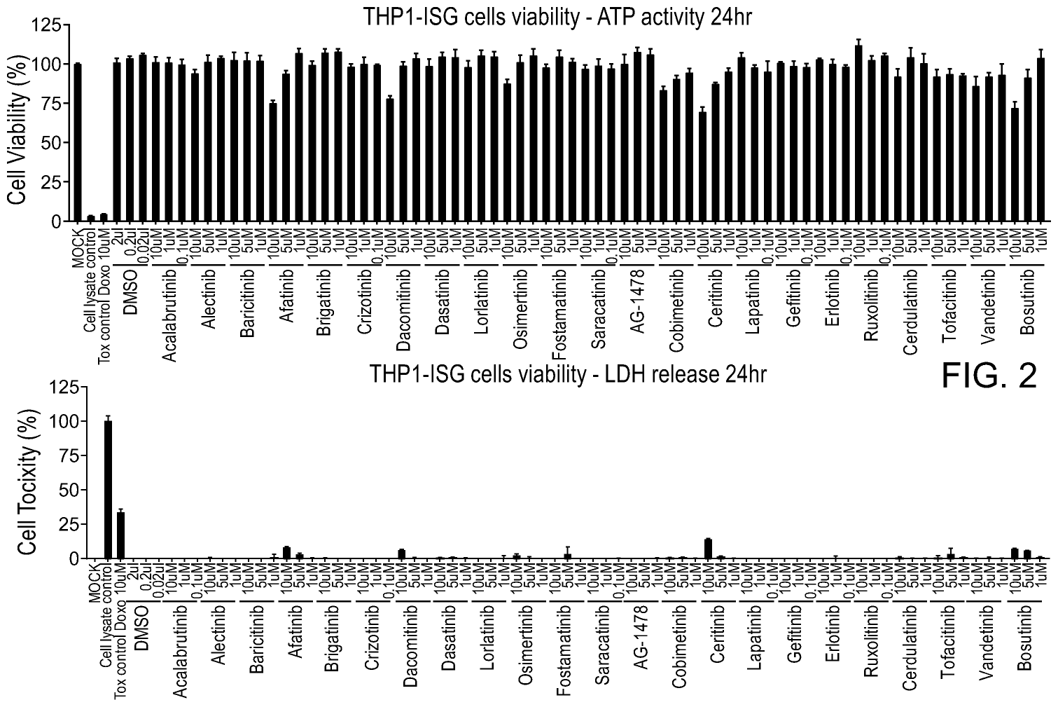

Toxicity of these compounds was evaluated at 5 μM and 10 μM by two complementary methods: ATP activity and LDH release at 24 hours (see FIG. 2). As shown in FIG. 2, most of these compounds showed low or no toxicity at the evaluated at 5 μM and 10 μM concentrations.

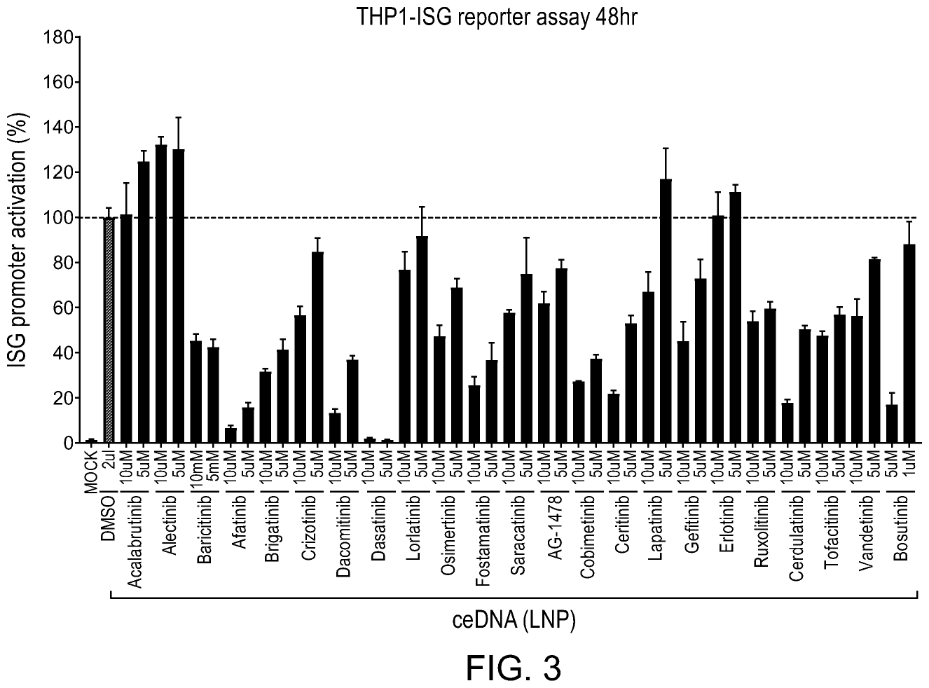

Using these concentrations in which no apparent toxicity was observed (5 μM and 10 μM), the compounds were tested assay for their effect on IFN reporter induction in THP1-ISG cells either in using ceDNA formulated in an LNP as an agonist and mock and DMSO negative controls. By 48 hours post induction, 10 out of 23 tested compounds effectively inhibited the IFN-1 response to ceDNA in THP1 cells either at 5 μM or 10 μM by attenuating IFN-1 reporter expression (ISG-luciferase) by more than 50% (>50% reduction) (FIG. 3).

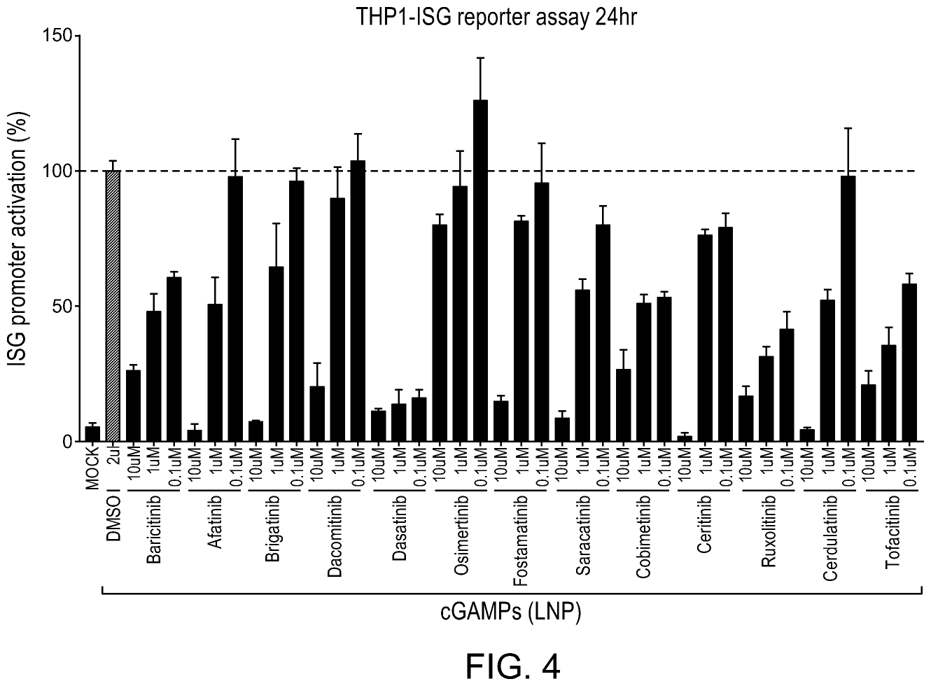

Using known STING agonists, cGAMPs, that activates cGAS/STING immunomodulating pathway were also used to demonstrate that the effect of the selected immunomodulators on the IFN responses to ceDNA was consistent with these known activators of cGAS/STING pathway. As shown in FIG. 4, most of these compounds also successfully attenuated IFN-1 responses triggered by the presence of cGAMPs that had been similarly formulated in LNPs in a dose dependent manner. Particularly, five out of fifteen compounds showed 50% or more reductions in STING activation at their 10 μM, 1 μM or 0.1 μM concentrations.

Based on the data above, it was suggested that the effect of ceDNA on induction of ISG could be elicited through the cGAS/STING pathway that recognizes cGAMPs. Furthermore, it was found that most of the compounds demonstrating inhibitory effects on ceDNA associated ISGs response were known tyrosine kinase inhibitors (TKIs).

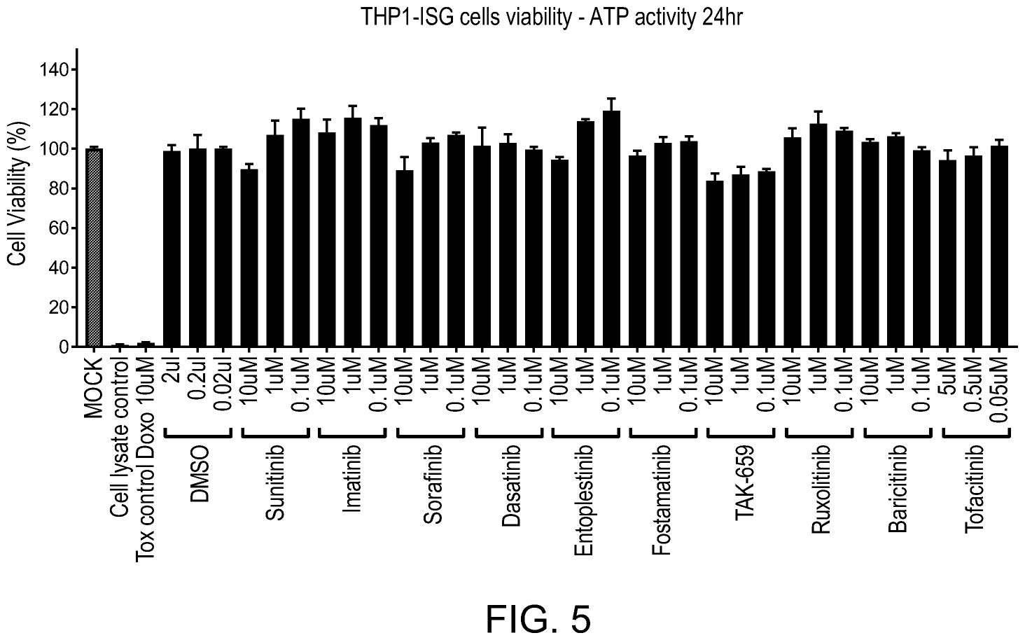

A model of TKIs being the effective antagonists of ceDNA-associated immune responses was validated using five additional known TKIs including sunitinib, imatinib, sorafinib, entoplestinib, and TAK-659. As shown in FIG. 5 and FIG. 6, these TKIs were mostly non-toxic in vitro at 10 μM, 1.0 μM and 0.1 μM concentrations. These TKIs, except imatinib, also successfully demonstrated the inhibitory effect on ISG transcriptional induction (>50% reductions in tested concentrations at 10 μM, 1.0 μM or 0.1 μM) like other TKIs that were previously tested (e.g., dasatinib, postamatinib, ruxolitinib, baricitinib, and tofacitinib).

Finally, IFN reporter assays were conducted in RAW-ISG cells. RAW-ISG cells are generated from the murine macrophage cell line by stable integration of an interferon regulatory factor (IRF)-inducible luciferase reporter construct. Hence, RAW-ISG cells also allow monitoring of IRF activation in this mouse cell line. As shown in FIG. 7, ruxolitinib and fostamatinib demonstrated significant inhibitory effects on the murine ISG system as they did in human cells (THP-1). Another potent TKI compound selected for this assay was BMS-986165 which is a known antagonist of TYK2. As shown in FIG. 7, BMS-986165 exhibited significant inhibitory effects on induction of IRF at all concentration tested (e.g., 10 M, 1.0 μM and 0.1 μM) (FIG. 7). These TKI compounds exerted consistent and significant inhibitory effects on the IFN response (ISG) to ceDNA in a dose dependent manner, confirming the previously observed immunosuppressant effects by these TKIs to the presence of nucleic acid therapeutics like ceDNA. Among more than 30 compounds tested, ruxolitinib, fostamatinib and BMS-986165 were showed most significant inhibitory effects in vivo.

Fostamatinib or fostamatinib disodium hexahydrate is a tyrosine kinase inhibitor with the demonstrated activity against spleen tyrosine kinase (SYK). Fostamatinib is used for the treatment of thrombocytopenia in adult patients with chronic immune thrombocytopenia (ITP) who have had an insufficient response to a previous treatment. The major metabolite of fostamatinib, R406, is also known to inhibit signal transduction of Fc-activating receptors and B-cell receptor and to reduce antibody-mediated destruction of platelets.

Ruxolitinib inhibits Janus Associated Kinases 1 and 2 (JAK1 and JAK2) with IC₅₀ at 3.3 nM and 2.8 nM respectively. JAKs mediate signaling of a number of cytokines and growth factors that play important roles in hematopoiesis and immune responses. JAK signaling involves recruitment of STATs (signal transducers and activators of transcription) to cytokine receptors, activation and subsequent localization of STATs to the nucleus leading to modulation of gene expression. Ruxolitinib is approved for the treatment of intermediate or high-risk myelofibrosis, including primary myelofibrosis, post-polycythemia vera myelofibrosis, and post-essential thrombocythemia myelofibrosis.

In summary, TKI compounds tested exhibited significant inhibitory effects on IFN response (≥50% inhibition of the ISG promoter activation), suggesting that some of the TKIs tested indeed possess important immunosuppressive effects in mammalian cells exposed to a therapeutic nucleic acid such as ceDNA. The primary targets of these compounds are potentially EGFR, ALK, Syk, and ABL/Src. Also, targeting the IFN production/signaling pathway with Jak inhibitors significantly inhibits ceDNA-mediated ISG promoter activation in THP1 cells. Targeting these molecules for inhibition may not only inhibit the innate immune response, but also delay and mitigate the cellular immune response with benefits in transgene expression and safety. Results showed that Syk inhibitor Fostamatinib and Jak1/2/Tyk inhibitor Ruxolitinib drastically reduce cytokines expression after ceDNA dosing (LNP: Construct A at 0.2 mg/Kg).

### Example 2. In Vivo Effects of Selected Tyrosine Kinase Inhibitors on Immune Response

To evaluate whether selected kinase inhibitors that showed beneficial properties in vitro can exert similar immunosuppressive effects in vivo, particularly in response to ceDNA, a group of TKIs were tested for their ability to suppress production of proinflammatory cytokines and chemokines. The immunosuppressant selected for initial in vivo cytokine/chemokine panel assays were afatinib, brigatinib, dacomitinib, dasatinib, saracatinib, fostamatinib, cerdulatinib, ruxolitinib, and cobimetinib. In experimental groups, rats were dosed with two different concentrations (high dose (H) and low (L) doses) of these compounds along with a ceDNA vector. ceDNA employed (“Construct A”) contains 5′ and 3′ ITRs and CpG minimized sequences for hAAT promoter operatively linked to a luciferase reporter gene. The high and low dose concentrations immunosuppressants are as follows: afatinib (H: 200 mg/kg/day; L: 25 mg/kg/day), brigatinib (H: 50 mg/kg/day; L: 10 mg/kg/day), dacomitinib (H: 30 mg/kg/day; L: 10 mg/kg/day), dasatinib (H: 30 mg/kg/day; L: 10 mg/kg/day), saracatinib (H: 50 mg/kg/day; L: 10 mg/kg/day), fostamatinib (H: 500 mg/kg/day; L: 100 mg/kg/day), cerdulatinib (H: 30 mg/kg/day; L: 5 mg/kg/day), ruxolitinib (H: 300 mg/kg/day; L: 60 mg/kg/day) and cobimetinib (H: 10 mg/kg/day; L: 3 mg/kg/day).

Formulations were mixed by pipetting prior to administration to distribute particulates of oral gavage suspension. TKI or vehicle for Groups 3-24 were dosed on Days −2, −1, 0 and 1 by oral gavage at 10 mL/kg. TKI for Group 25 were dosed on Days −2, −1, 0 and 1 by oral gavage at 20 mL/kg. TKI for Group 26 was dosed on Days −2, −1, 0 and 1 by intraperitoneal (IP) administration at 20 mL/kg. On day 0, TKIs were dosed 1.5 hours (10 minutes) prior to the ceDNA administration. Otherwise, TKIs were administered at approximately the same time each day (f 1 hour). ceDNA was dosed at 0.2 mg/kg on Day 0 for all experimental groups by intravenous administration via lateral tail vein.

For in-life imaging (IVIS), animals in Groups 1-26 were dosed with luciferin at 150 m/kg (60 mg/mL) via intraperitoneal (IP) injection at 2.5 mL/kg less than 15 minutes post each luciferin administration; all animals had an IVIS imaging session on days 6, 14, 21, 28, and 35. The results of the IVIS imaging were described below.

Animals were monitored continuously while under anesthesia, during recovery and until mobile. All animals in Groups 1-26 had interim blood collected on Day 0; 6 hours post Test material dose (±5%).

Expression levels of ceDNA as detected by luciferase (IVIS for luciferase) was measured at day 6, 14, 21, 28, and 35 in all groups (FIG. 8). Polycytidylic acid potassium salt (PolyC) and naked ceDNA (Construct A) were used as controls. Both polyC and Construct A were formulated in LNP and used as controls for in groups in which no ceDNA was dosed to evaluate the effect of LNPs. Body weights for all animal were recorded.

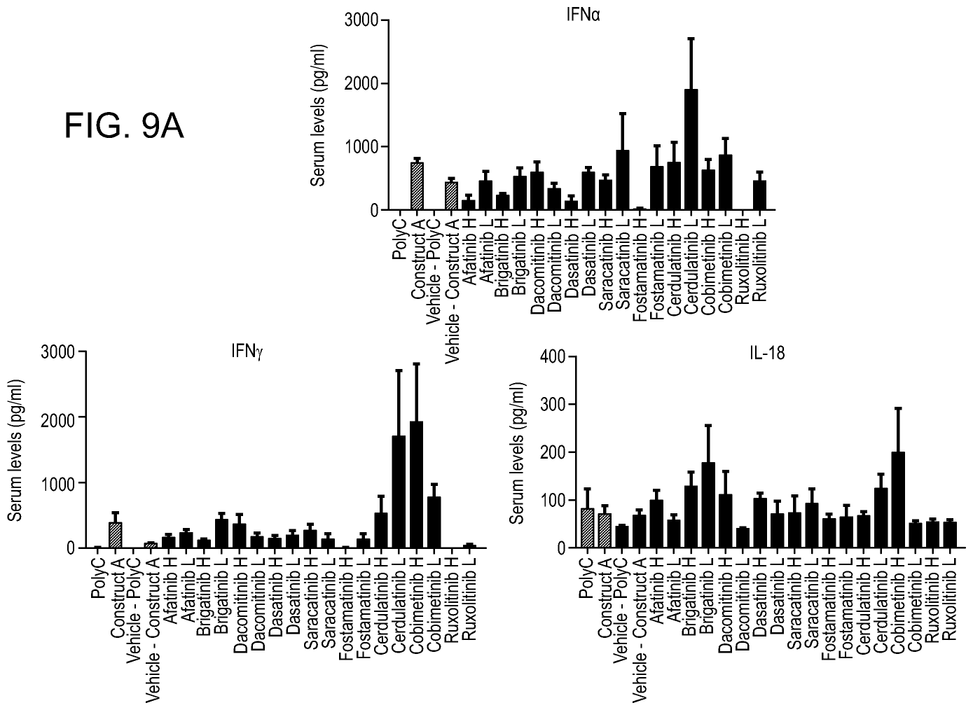

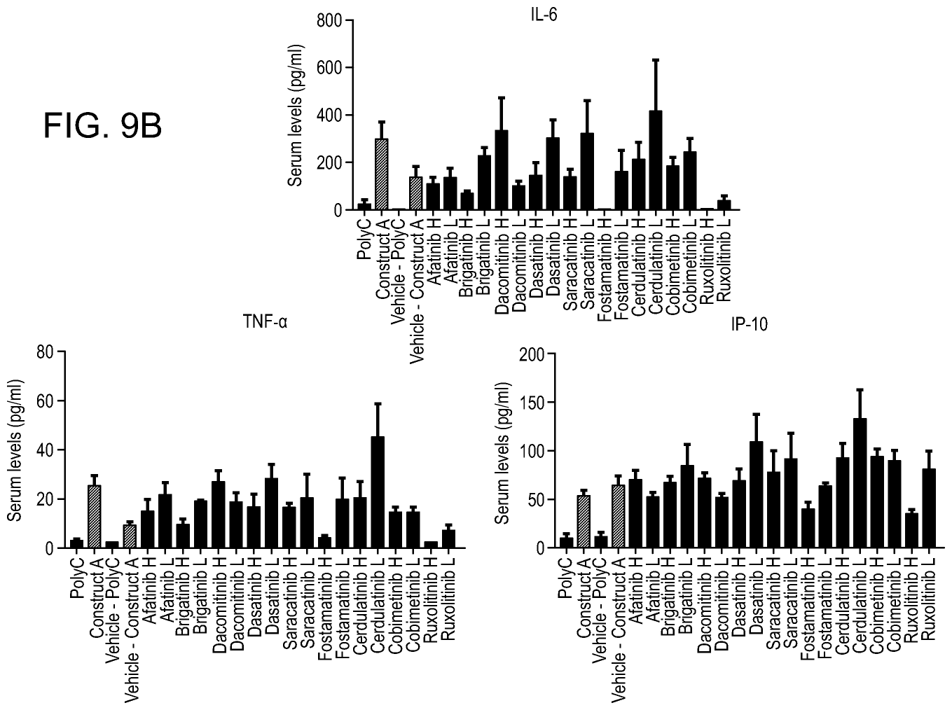

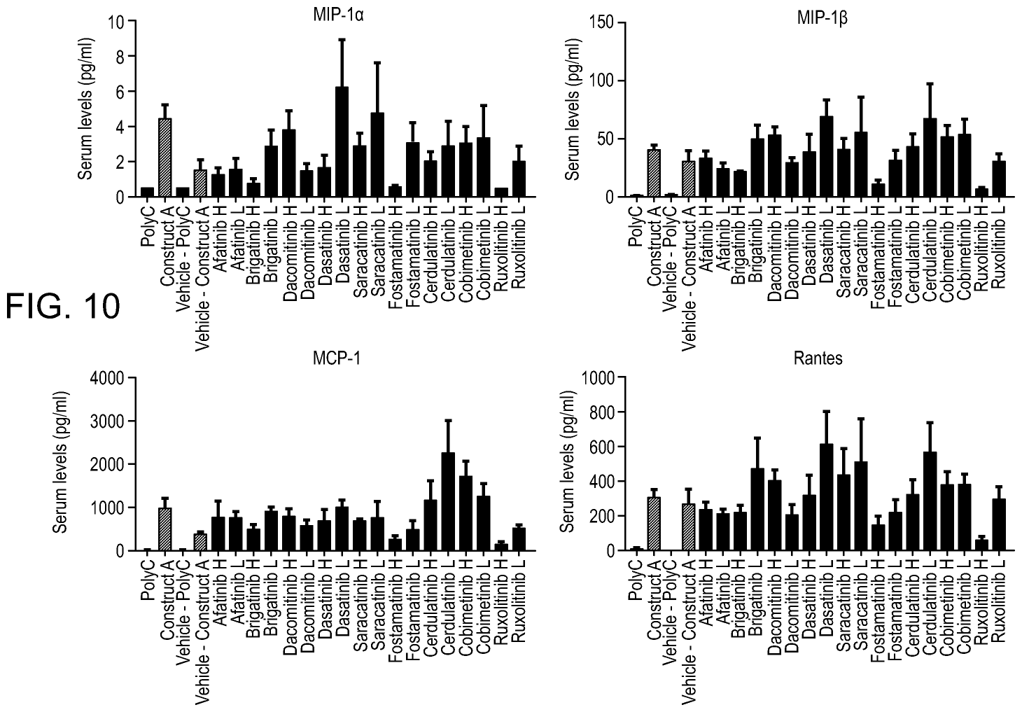

All compounds were well tolerated without triggering any adverse reaction in response to 0.2 mg/kg of ceDNA. To note, while the group treated with ruxolitinib demonstrated a certain level of weight gains, suggesting significant tolerability of this TKI in conjunction with ceDNA, approximately 7-8% body loss was observed in the group treated with afatinib and ceDNA, particularly at a high dose. Furthermore, ceDNA expression as observed by high luciferase expression at the given dose (0.2 mg/kg) was also seen in all study group carrying the ceDNA vector. In particular, the group dosed with ruxolitinib exhibited the highest level of ceDNA transgene expression levels at day 6 (FIG. 8). Fostamatinib and ruxolitinib stood out for their effects on suppressing cytokines and chemokine levels (FIGS. 9A and 9B). In particular, animals treated with ruxolitinib exhibited significant reductions in their serum cytokine levels including interferon-α (IFN-α), interferon-γ (IFN-γ), tumor necrosis factor-α (TNF-α), interleukin-10 (IL-1p), interleukin-6 (IL-6), interleukin-18 (IL-18) (see FIGS. 9A and 9B). Similarly, these animals treated with ruxolitinib also exhibited significant reduction in their serum chemokine levels including MCP-1, MIP-1α (CCL3), MIP-1β (CCL4), and Rantes (CCL5) (see FIG. 10). Further, it appeared that the LNP delivery seemed to attenuate (30-40% reduction) proinflammatory reaction against ceDNA in vivo as shown in IFN-α levels of Construct A compared to that of Construct A:LNP vehicle. Surprisingly, the group treated with a high dose of ruxolitinib with Construct A:LNP vehicle showed no detectable IFN-α, IFN-γ, and IL-6 levels in their serum (see FIGS. 9A and 9B). Both compounds effectively attenuated type I and type II IFNs responses to baseline.

Further, the in vivo proinflammatory experiments were repeated using ruxolitinib and fostamatinib to confirm the observation described above. As shown in FIGS. 11A and 11B, these two TKIs successfully exerted immunosuppressive effects in vivo in response to 0.2 mg/kg ceDNA as compared to untreated animal groups (LNP vehicle with polyC or ceDNA alone) in all cytokines measured (IFN-α, IFN-γ, TNF-α, IL-6, IL-18 and IP-10).

Dose Escalation Study

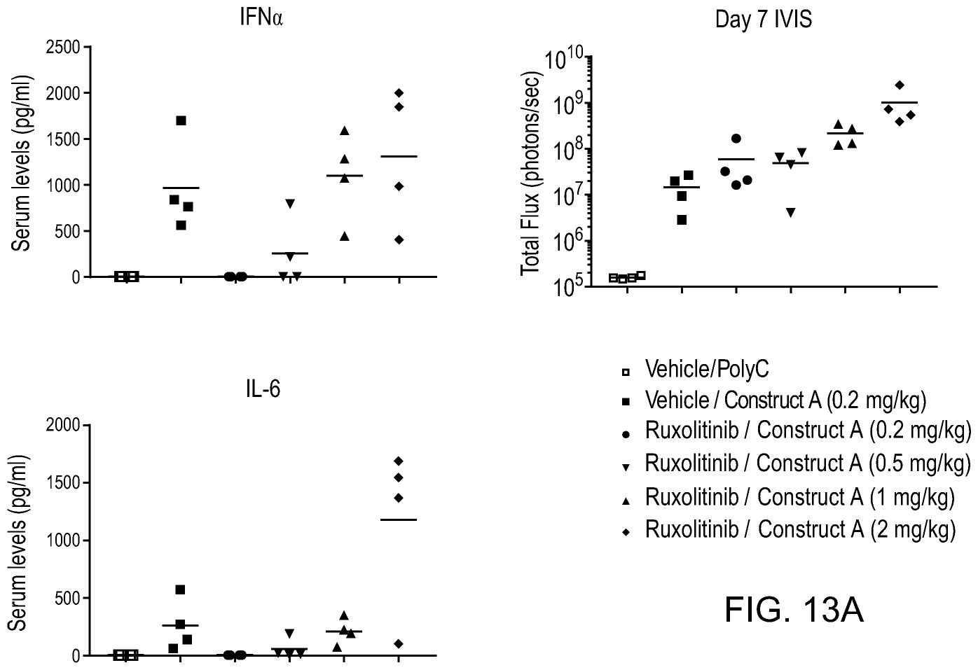

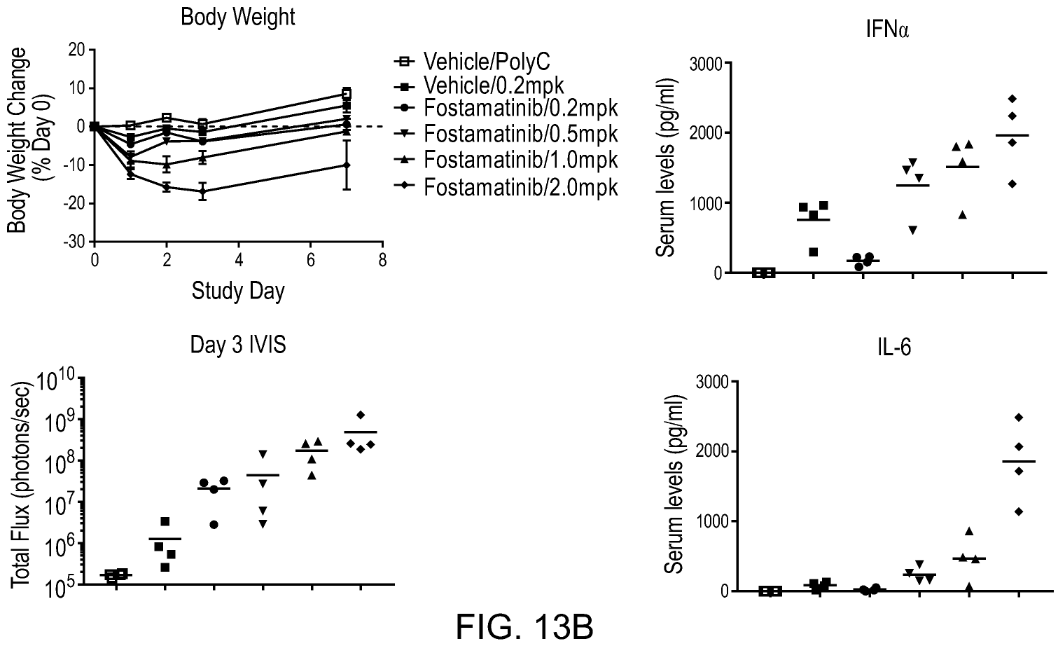

An in vivo study with dose escalation of ceDNA was conducted to evaluate the effect of ceDNA at a high dose in the presence of the TKI (ruxolitinib at a high dose of 300 mg/kg and fostamatinib at a high dose of 500 mg/kg). In this study, four experimental groups were dosed with increasing doses of 0.2 mg/kg, 0.5 mg/kg, 1 mg/kg, or 2 mg/kg of ceDNA (Construct A: hAAT-Luciferase) formulated in LNPs. As shown in FIGS. 13A and 13B, expression of ceDNA increased in a dose dependent manner as seen in day 7 IVIS. Surprisingly, the animal group dosed with a high amount of ceDNA (2 mg/kg) formulated in LNP well tolerated the treatment and exhibited serum cytokine levels that were similar to the ones seen in 10-fold less ceDNA (0.2 mg/kg) without the treatment with ruxolitinib (FIG. 13A), suggesting that ruxolitinib could potentially provide at least 10-fold greater therapeutic benefit in gene or nucleic acid therapy.

TKI Dosing Timelines

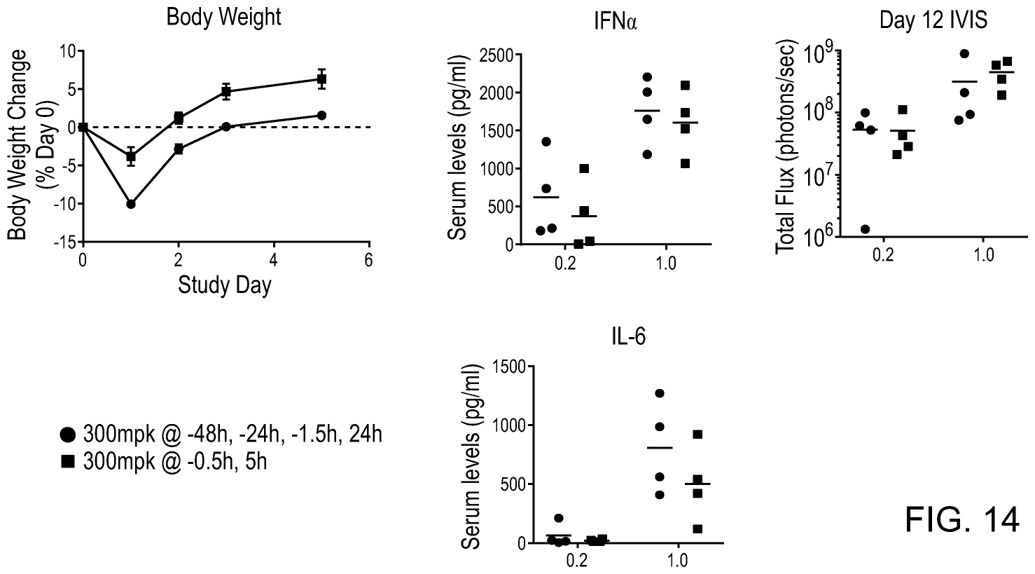

FIG. 14 depicts two different dosing time arrangements of TKI treatment. The first group of mice (n=4) was dosed with 300 mg/kg ruxolitinib at −48 h, −24 h, −1.5 h, and 24 hr in relation to ceDNA administration at 0 hr with at either 0.2 mg/kg or 1.0 mg/kg. The second group of mice (n=4) was dosed with 300 mg/kg ruxolitinib at −0.5 h and 5 h in relation to ceDNA administration at 0 hr at either 0.2 mg/kg or 1.0 mg/kg. All animals well tolerated the treatment as they flourished with increasing body weight. As expected, day 12 IVIS showed dose dependent expression of ceDNA (FIG. 14). Dosing timeline of ruxolitinib with the −0.5 h and 5 h arrangement apparently had an improved immunosuppression as compared to a more spread out and more frequent dosage regimen of −48 hr, −24 h, −1.5 h, and 24 h, suggesting that TKIs like ruxolitinib can be highly effective when they are administered more close in time with ceDNA administration.

Factor IX ceDNA Dosing In Vivo-Preliminary Study

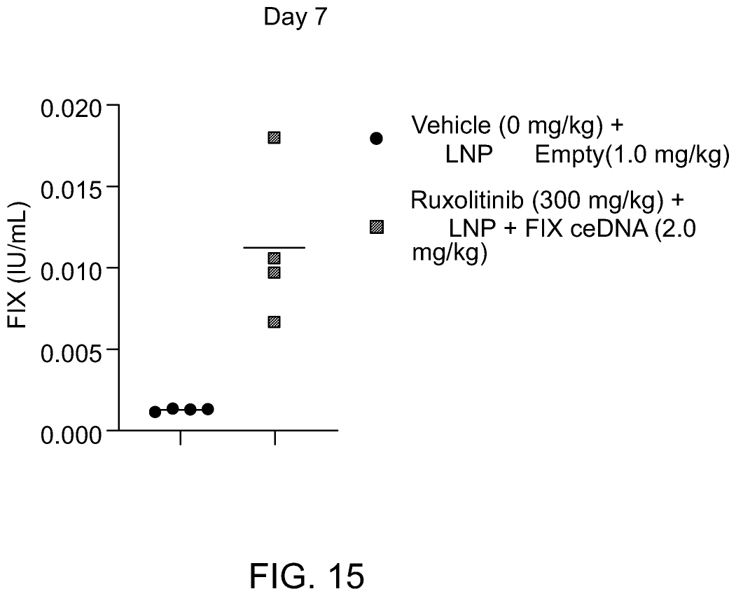

In a first study, a therapeutic ceDNA carrying human Factor IX (FIX) was dosed in mice (n=4) to evaluate the effect of ruxolitinib and in vivo expression of FIX. Experimental conditions were similar to those implemented in in vivo analyses described above. As shown in FIG. 15, FIX protein was successfully expressed and detected in vivo at day 7 in animals treated with 300 mg/kg of ruxolitinib at −48 h, −24 h, −1.5 h, and 24 hr and a high concentration of 2.0 mg/kg FIX ceDNA at day 0, suggesting that the combination approach of the present invention can be successfully applied in a therapeutic model.

In Vivo Re-Dosing

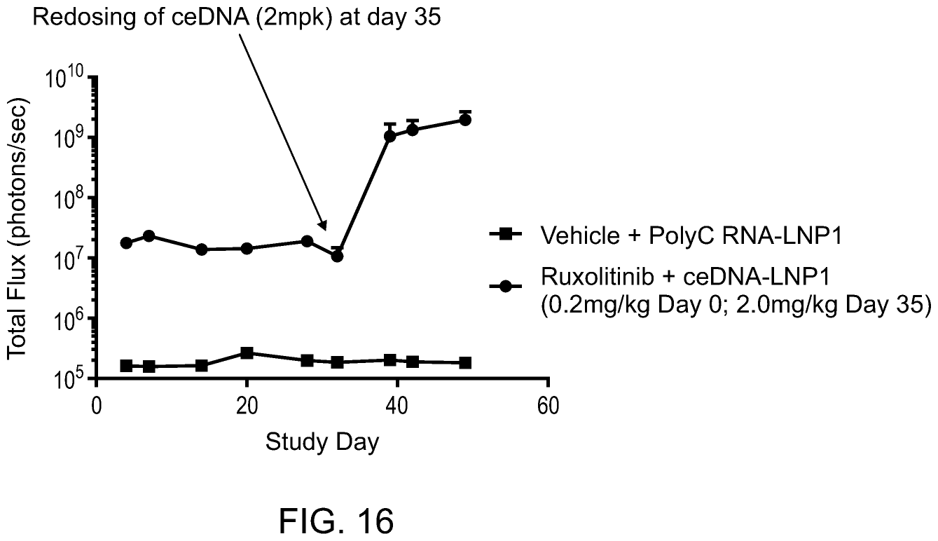

To further evaluate whether the combination approaches of the present invention allow for re-dosing of a therapeutic nucleic acid such as ceDNA, ceDNA carrying a report construct (Construct A: hAAT-Luc) was dosed at 0.2 mg/kg at day 0 and re-dosed at a 10-fold higher concentration at day 35 in mice treated with 300 mg/kg ruxolitinib at −48 h, −24 h, −1.5 h, and 24 hr in relation to each ceDNA dose (day 0 and day 35). As shown in FIG. 16, the initial dose of ceDNA resulting in continued expression up to 35 days at a therapeutically meaningful level. Surprisingly, re-dosing of ceDNA at day 35 drastically increased the level of expression beyond day 35, suggesting that immunosuppressant TKIs can be implemented in a repeated dose regimen of nucleic acid therapy like ceDNA to intensify expression of a therapeutic nucleic acid.

Factor IX Therapeutic ceDNA Dosing and Re-Dosing In Vivo Extended Study

In an extended study, a therapeutic ceDNA carrying human Factor IX (FIX) was dosed in mice (n=4) to evaluate the effect of the combination of an immunosuppressant TKI (e.g., ruxolitinib) and a therapeutic FIX nucleic acid on in vivo expression of FIX over a period of 56 days. The study design and details were carried out as set forth below.

Study Design

Table 2 sets forth the design of the kinase inhibitor administration component of the study. As shown in Table 2, two groups of male CD-1 mice (Group 1, n=4; Group 2, n=4) were orally administered either vehicle or ruxolitinib (300 mg/kg) at a dose volume of 10 mL/kg. For both Groups 1 and 2 dosing was carried out at days −2, −1, 1, 0 and 36.

TABLE 2

| | Animals | | Dose | Dose | |
|---|---|---|---|---|---|
| Group | per | | Level | Volume | Treatment Regimen, |
| No. | Group | Inhibitor ^{a} | (mg/kg) | (mL/kg) | via PO |
| 1 | 4 | Vehicle | NA | 10 | Days −2, −1 & 1 |
| | | | | | Day 0: 90 min. pre-dose |
| | | | | | Day 36: 30 min. pre-dose |
| | | | | | & 5 hours post dose |
| 2 | 4 | Ruxolitinib | 300 | | |

No. = Number;
PO = oral gavage;
ROA = route of administration;
min = minutes;
hrs = hours.
^{a} Vehicle for dosing and inhibitor preparation = 0.5% methylcellulose

Table 3 sets forth the design of the test material administration component of the study. As shown in Table 3, one group of male CD-1 mice (Group 1, n=4) was intravenously administered either LNP:Empty on day 0 or LNP:Empty on day 36 at a dose level of 1 mg/kg and a dose volume of 5 mL/kg. The second group of male CD-1 mice (Group 2, n=4) was intravenously administered with ceDNA carrying a human Factor IX expression cassette formulated in LNP (LNP:ceDNA-FIX) on day 0 and re-dosed with LNP:ceDNA-FIX on day 36 at a dose level of 2 mg/kg and a dose volume of 5 mg/kg. Day 56 was the terminal time point of the study.

TABLE 3

| | Animals | | Dose | Dose | Treatment | Terminal |
|---|---|---|---|---|---|---|
| Group | per | | Level | Volume | Regimen, | Time |
| No. | Group | Treatment | (mg/kg) | (mL/kg) | IV | Point |
| 1 | 4 | LNP:Empty | 1.0 | 5 | Once on | Day 56 |
| | | (Day 0) or | | | Day 0 & | |
| | | LNP:Empty | | | 36 | |
| | | (Day 36) | | | | |
| 2 | 4 | LNP:ceDNA- | 2.0 | | | |
| | | FIX | | | | |
| | | LNP:ceDNA- | | | | |
| | | FIX (Day 36) | | | | |

No. = Number; IV = intravenous; ROA = route of administration

Sample Collection

Blood collection or plasma collection (interim) was carried out as follows. For mice in both Groups 1 and 2, a sample of 120 μl of whole blood was collected orbitally on days 7, 14, 21, 28, 35, 42, 49, and 56. A plasma sample was collected from the blood samples collected on days 7, 14, 21, 28, 35, 42, 49 and 56. To process and store the blood samples, 120 μL of whole blood was added to a tube pre-coated with 13.33 μL of 3.2% sodium citrate and kept ambient until processed. To process and store the plasma samples, one aliquot of plasma was frozen at nominally −70° C.

Study Details

Body Weights: Body weights for all animals were be recorded on Days −2, −1, 0, 1, 2, 3, 7, 14, 21, 28, 35, 36, 37, 38, 39, 42, 49, and 56 (prior to euthanasia). Additional body weights were recorded as needed.

Interim Blood Collection: All animals in Groups 1-2 had interim blood collected on Day 0 at 6 hours post Test Material dose (±5%); then on Day 7, 14, 21, 28, 35, 42, 49, and 56 as indicated above.

Inhibitor Administration: Inhibitor or vehicle was dosed on Days −2, −1, 0 & 1 and again on Day 36 by PO administration (oral gavage) at 10 mL/kg. On Day 0, inhibitor or vehicle was dosed 1.5 hours (f 10 minutes) prior to the Day 0 ceDNA administration. On Day 36, inhibitor or vehicle was be dosed 0.5 hours (f 10 minutes) prior to the Day 0 ceDNA administration and 5 hours (f 20 minutes) post administration. Inhibitors were administered at approximately the same time each day (±1 hour).

Dose Administration: Test articles were be dosed at 5 mL/kg on Day 0 and Day 36 for Groups 1-2 by intravenous (IV) administration via lateral tail vein.

Euthanasia & Terminal Collection: On Day 56, after bleed for plasma, animals were euthanized by CO₂ asphyxiation followed by thoracotomy or cervical dislocation. No tissues will be collected.

As shown in FIG. 19, mice treated with ruxolitinib (300 mg/kg) at days −2, −1, 1, 0 and 36 and LNP:ceDNA-FIX (2.0 mg/kg) at day 0 and day 36 expressed factor IX (FIX) protein (IU/mL) that was detected in vivo beginning at day 7 through the end of the study (day 56). Notably, re-dosing with ceDNA-FIX at day 36 resulted in a dramatic increase in FIX expression beyond day 42 to the end of the study. In contrast, mice treated with vehicle control at −48 h, −24 h, −1.5 h, and 24 hr and LNP:empty (1.0 mg/kg) at day 0 or day 36 did not express FIX protein.

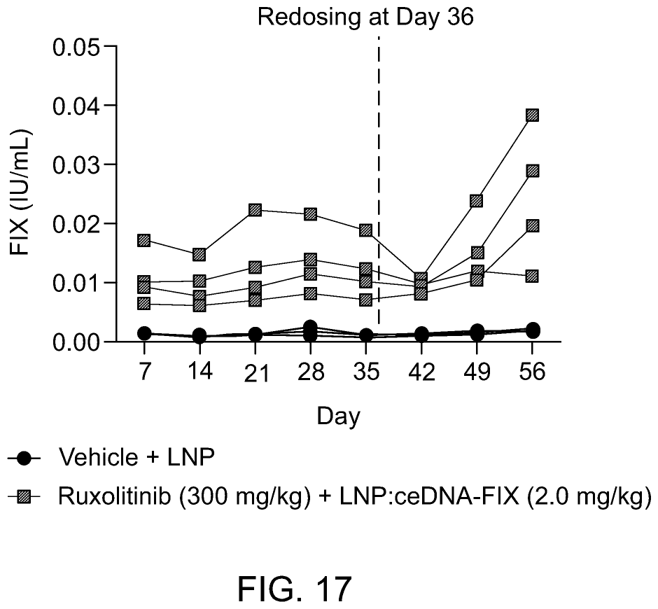

These results also demonstrate that a therapeutic nucleic acid (e.g., ceDNA-FIX) can be administered and re-dosed multiple times in conjunction with one or more immunosuppressant TKIs (e.g. the JAK inhibitor ruxolitinib) in a therapeutic model. As shown in FIG. 17, the combination approach allowed for a re-dosing of ceDNA-FIX, which led to a considerable increase in FIX expression.

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

What is claimed is:

1. A pharmaceutical composition comprising a therapeutic nucleic acid (TNA) and a tyrosine kinase inhibitor (TKI).

2. The pharmaceutical composition of claim 1, wherein the therapeutic nucleic acid is an RNA molecule, or a derivative thereof.

3. The pharmaceutical composition of claim 2, wherein the RNA molecule is an antisense oligonucleotide.

4. The pharmaceutical composition of claim 3, wherein the antisense oligonucleotide is an antisense RNA.

5. The pharmaceutical composition of claim 3, wherein the RNA is RNA interference (RNAi).

6. The pharmaceutical composition of claim 1, wherein the therapeutic nucleic acid is an mRNA molecule.

7. The pharmaceutical composition of claim 1, wherein the therapeutic nucleic acid is a DNA molecule, or a derivative thereof.

8. The pharmaceutical composition of claim 1, wherein the therapeutic nucleic acid is a DNA antisense oligonucleotide.

9. The pharmaceutical composition of claim 8, wherein the DNA antisense oligonucleotide is morpholino based nucleic acid.

10. The pharmaceutical composition of claim 9, wherein the morpholino based nucleic acid is a phosphorodiamidate morpholino oligomer (PMO).

11. The pharmaceutical composition of claim 1, wherein the therapeutic nucleic acid is a closed-ended DNA (ceDNA).

12. The pharmaceutical composition of claim 11, wherein the ceDNA comprises an expression cassette comprising a promoter sequence and a transgene.

13. The pharmaceutical composition of claim 12, wherein the ceDNA comprises an expression cassette comprising a polyadenylation sequence.

14. The pharmaceutical composition of claim 12, wherein the ceDNA comprises at least one inverted terminal repeat (ITR) flanking either 5′ or 3′end of said expression cassette.

15. The pharmaceutical composition of claim 12, wherein said expression cassette is flanked by two ITRs, wherein the two ITRs comprise one 5′ ITR and one 3′ ITR.

16. The pharmaceutical composition of claim 12, wherein the expression cassette is connected to an ITR at 3′ end (3′ ITR).

17. The pharmaceutical composition of claim 12, wherein the expression cassette is connected to an ITR at 5′ end (5′ ITR).

18. The pharmaceutical composition of claim 11, wherein the ceDNA further comprises a spacer sequence between a 5′ ITR and the expression cassette.

19. The pharmaceutical composition of claim 11, wherein the ceDNA further comprises a spacer sequence between a 3′ ITR and the expression cassette.

20. The pharmaceutical composition of any one of claims 18 and 19, wherein the spacer sequence is at least 5 base pairs long in length.

21. The pharmaceutical composition of claim 20, wherein the spacer sequence is 5 to 200 base pairs long in length.

22. The pharmaceutical composition of claim 20, wherein the spacer sequence is 5 to 500 base pairs long in length.

23. The pharmaceutical composition of claim 11, wherein the ceDNA has a nick or a gap.

24. The pharmaceutical composition of any one of claims 11 to 23, wherein the ceDNA is synthetically produced in a cell-free environment.

25. The pharmaceutical composition of any one of claims 11 to 23, wherein the ceDNA is produced in a cell.

26. The pharmaceutical composition of any one of claims 11 to 23 or 25, wherein the ceDNA is produced in insect cells.

27. The pharmaceutical composition of claim 26, wherein the insect cell is Sf9.

28. The pharmaceutical composition of claim 25, wherein the ceDNA is produced in a mammalian cell.

29. The pharmaceutical composition of claim 28, wherein the mammalian cell is a human cell line.

30. The pharmaceutical composition of claim 14, wherein the ITR is an ITR derived from an AAV serotype.

31. The pharmaceutical composition of any one of claims 14 to 19, wherein said AAV is selected from the group consisting of AAV1, AAV2, AAV3, AAV4, AAV5, AAV6, AAV7, AAV8, AAV9, AAV10, AAV11 and AAV12.

32. The pharmaceutical composition of any one of claims 14 to 19, wherein the ITR is derived from an ITR of goose virus.

33. The pharmaceutical composition of any one of claims 14 to 19, wherein the ITR is derived from a B19 virus ITR.

34. The pharmaceutical composition of any one of claims 14 to 19, wherein the ITR is a wild-type ITR from a parvovirus.

35. The pharmaceutical composition of any one of claims 14 to 19, wherein the ITR is a mutant ITR, and the ceDNA optionally comprises an additional ITR which differs from the first ITR.

36. The pharmaceutical composition of claim 35, wherein the ceDNA comprises two mutant ITRs in both 5′ and 3′ends of the expression cassette, optionally wherein the two mutant ITRs are symmetric mutants.

37. The pharmaceutical composition of claim 1, wherein the therapeutic nucleic acid is a DNA-based minicircle or a MIDGE.

38. The pharmaceutical composition of claim 1, wherein the therapeutic nucleic acid is a linear covalently closed-ended DNA vector.

39. The pharmaceutical composition of claim 38, wherein the linear covalently closed-ended DNA vector is a ministring DNA.

40. The pharmaceutical composition of claim 1, wherein the therapeutic nucleic acid is a closed-ended DNA comprising at least one protelomerase target sequence in the 5′ and 3′ ends of the expression cassette.

41. The pharmaceutical composition of claim 1, wherein the therapeutic nucleic acid is a dumbbell shaped linear duplex closed-ended DNA comprising two hairpin structures of ITRs in the 5′ and 3′ ends of an expression cassette.

42. The pharmaceutical composition of claim 1, wherein the therapeutic nucleic acid is a doggybone (dbDNA™) DNA.

43. The pharmaceutical composition of claim 1, wherein the therapeutic nucleic acid is a minigene.

44. The pharmaceutical composition of claim 1, wherein the therapeutic nucleic acid is a plasmid.

45. The pharmaceutical composition of any one of claims 1 to 44, wherein the tyrosine kinase inhibitor is a pharmaceutically acceptable salt of the tyrosine kinase inhibitor.

46. The pharmaceutical composition of any one of claims 1 to 44, wherein the composition further comprises an excipient or carrier.

47. The pharmaceutical composition of any one of claims 1 to 44, wherein the pharmaceutical composition comprises a lipid nanoparticle (LNP).

48. The pharmaceutical composition of any one of claims 1 to 47, wherein the TKI is selected from the group consisting of acalabrutinib, alectinib, baricitinib, afatinib, brigatinib, crizotinib, dacomitinib, dasatinib, lorlatinib, osimertinib, fostamatinib, saracatinib, AG-1478, cobimetinib, ceritinib, lapatinib, gefitinib, erlotinib, ruxolitinib, cerdulatinib, tofacitinib, BMS-986165, vandetinib, and bosutinib.

49. The pharmaceutical composition of any one of claims 1 to 47, wherein the TKI is selected from the group consisting of baricitinib, afatinib, brigatinib, dacomitinib, dasatinib, osimertinib, fostamatinib, saracatinib, cobimetinib, ceritinib, ruxolitinib, cerdulatinib, BMS-986165, and tofacitinib.

50. The pharmaceutical composition of any one of claims 1 to 47, wherein the TKI is selected from the group consisting of sunitinib, imatinib, sorafenib, dasatinib, entoplestinib, fostamatinib, TAK-659, ruxolitinib, baricitinib, BMS-986165, and tofacitinib.

51. The pharmaceutical composition of claim 1, wherein the TKI is an inhibitor of STAT1.

52. The pharmaceutical composition of claim 1, wherein the TKI is an inhibitor of STAT2.

53. The pharmaceutical composition of claim 1, wherein the TKI is an inhibitor of spleen tyrosine kinase (Syk).

54. The pharmaceutical composition of claim 53, wherein the Syk inhibitor is fostamatinib.

55. The pharmaceutical composition of claim 53, wherein the Syk inhibitor is cerdulatinib.

56. The pharmaceutical composition of claim 1, wherein the TKI is an inhibitor of epidermal growth factor receptor (EGFR, aka ErbB-1 or HER-1).

57. The pharmaceutical composition of claim 56, wherein the EGFR inhibitor is afatinib.

58. The pharmaceutical composition of claim 56, wherein the EGFR inhibitor is dacomitinib.

59. The pharmaceutical composition of claim 1, wherein the TKI is an inhibitor of anaplastic lymphoma kinase (ALK).

60. The pharmaceutical composition of claim 59, wherein the ALK inhibitor is brigatinib.

61. The pharmaceutical composition of claim 59, wherein the ALK inhibitor is alectinib.

62. The pharmaceutical composition of claim 59, wherein the ALK inhibitor is ceritinib.

63. The pharmaceutical composition of claim 59, wherein the ALK inhibitor is lorlatinib.

64. The pharmaceutical composition of claim 59, wherein the aid ALK inhibitor is crizotinib.

65. The pharmaceutical composition of claim 1, wherein the TKI is an antagonist of IFN production pathway.

66. The pharmaceutical composition of claim 1, wherein the TKI is an antagonist of IFN signaling pathway.

67. The pharmaceutical composition of claim 1, wherein the TKI is an inhibitor of tyrosine kinase 2 (Tyk2).

68. The pharmaceutical composition of claim 1, wherein the TKI is an inhibitor of Janus kinase 1 (Jak1).

69. The pharmaceutical composition of claim 1, wherein the TKI is an inhibitor of Janus kinase 2 (Jak2).

70. The pharmaceutical composition of claim 67, wherein the Tyk2 inhibitor is ruxolitinib.

71. The pharmaceutical composition of claim 67, wherein the Tyk2 inhibitor is tofacitinib.

72. The pharmaceutical composition of claim 67, wherein the Tyk2 inhibitor is BMS-986165.

73. The pharmaceutical composition of claim 68, wherein the Jak1 inhibitor is ruxolitinib.

74. The pharmaceutical composition of claim 69, wherein the Jak2 inhibitor is ruxolitinib.

75. The pharmaceutical composition of claim 68, wherein the Jak1 inhibitor is baricitinib.

76. The pharmaceutical composition of claim 69, wherein the Jak2 inhibitor is baricitinib.

77. The pharmaceutical composition of claim 1, wherein the TKI is a Jak1/2 inhibitor selected from the group consisting of ruxolitinib, baricitinib, and tofacitinib.

78. The pharmaceutical composition of claim 1, wherein the TKI is Abl/Src inhibitor.

79. The pharmaceutical composition of claim 78, wherein the Abl/Src inhibitor is bosutinib.

80. A method of treating a genetic disorder in a subject, the method comprising administering to the subject an effective amount of the pharmaceutical composition according to any of claims 1-79.

81. The method of claim 80, wherein the subject is a human.

82. The method of claim 80, wherein the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson's disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), omithine transcarbamylase (OTC) deficiency, Usher syndrome, alpha-1 antitrypsin deficiency, progressive familial intrahepatic cholestasis (PFIC), and Cathepsin A deficiency.

83. The method of claim 82, wherein the genetic disorder is Leber congenital amaurosis (LCA).

84. The method of claim 83, wherein the LCA is LCA10.

85. The method of claim 82, wherein the genetic disorder is Niemann-Pick disease.

86. The method of claim 82, wherein the genetic disorder is Stargardt macular dystrophy.

87. The method of claim 82, wherein the genetic disorder is glucose-6-phosphatase (G6Pase) deficiency (glycogen storage disease type I) or Pompe disease (glycogen storage disease type II).

88. The method of claim 82, wherein the genetic disorder is hemophilia A (Factor VIII deficiency).

89. The method of claim 82, wherein the genetic disorder is hemophilia B (Factor IX deficiency).

90. The method of claim 82, wherein the genetic disorder is hunter syndrome (Mucopolysaccharidosis II).

91. The method of claim 82, wherein the genetic disorder is cystic fibrosis (CFTR).

92. The method of claim 82, wherein the genetic disorder is dystrophic epidermolysis bullosa (DEB).

93. The method of claim 82, wherein the genetic disorder is phenylketonuria (PKU).

94. The method of claim 82, wherein the genetic disorder is hyaluronidase deficiency.

95. A method of treating a genetic disorder in a subject, the method comprising administering to the subject an effective amount of a protein kinase inhibitor (PKI) and an effective amount of a therapeutic nucleic acid (TNA).

96. The method of claim 95, wherein the TNA is an RNA molecule, or a derivative thereof.

97. The method of claim 96, wherein the RNA molecule is an antisense oligonucleotide.

98. The method of claim 97, wherein the antisense oligonucleotide is an antisense RNA.

99. The method of claim 95, wherein the TNA is an RNA interference molecule.

100. The method of claim 95, wherein the TNA is an mRNA molecule.

101. The method of claim 95, wherein the TNA is a DNA molecule or a derivative thereof.

102. The method of claim 101, wherein the TNA is peptide nucleic acid (PNA), locked nucleic acid (LNA), or morpholino based antisense oligomer.

103. The method of claim 102, wherein the TNA is a DNA antisense oligonucleotide.

104. The method of claim 103, wherein the DNA antisense oligonucleotide is a morpholino based nucleic acid.

105. The method of claim 104, wherein the morpholino based nucleic acid is a phosphorodiamidate morpholino oligomer (PMO).

106. The method of claim 101, wherein the DNA is a closed-ended DNA (ceDNA).

107. The method of claim 106, wherein the ceDNA comprises an expression cassette comprising a promoter sequence operatively linked to a transgene.

108. The method of claim 107, wherein the ceDNA comprises expression cassette comprising a polyadenylation sequence.

109. The method of claim 107, wherein the ceDNA comprises at least one inverted terminal repeat (ITR) flanking either 5′ or 3′ end of the expression cassette.

110. The method of claim 109, wherein one ITR is connected to 5′ end (5′ ITR) and another ITR is connected to 3′ ends of the expression cassette.

111. The method of claim 109, wherein the ceDNA further comprises a spacer sequence between the 5′ ITR and the expression cassette.

112. The method of claim 109, wherein the ceDNA further comprises a spacer sequence between 3′ ITR and the expression cassette.

113. The method of any one of claims 111 and 112, wherein the spacer sequence is at least 5 base pairs long in length.

114. The method of claim 113, wherein the spacer sequence is 5 to 200 base pairs long in length.

115. The method of any one of claims 111 and 112, wherein the spacer sequence is 5 to 500 base pairs long in length.

116. The method of claim 109, wherein the ceDNA has a gap in one strand.

117. The method of any one of claims 106 to 116, wherein the ceDNA is synthetically produced in a cell-free environment.

118. The method of claim any one of claims 106 to 116, wherein the ceDNA is produced in a cell.

119. The method of claim 118, wherein the cell is an insect cell.

120. The method of claim 119, wherein the insect cell is Sf9.

121. The method of any one of claims 109 to 120, wherein the ITR is an ITR derived from an AAV serotype.

122. The method of claim 121, wherein the AAV serotype is selected from the group consisting of AAV1, AAV2, AAV3, AAV4, AAV5, AAV6, AAV7, AAV8, AAV9, AAV10, AAV11, and AAV12.

123. The method of claim 109, wherein the ITR is derived from an ITR of goose virus.

124. The method of claim 109, wherein the ITR is derived from a B19 virus ITR.

125. The method of claim 109, wherein the ITR is a wild-type ITR derived from a parvovirus.

126. The method of claim 109, wherein the ITR is a mutant ITR.

127. The method of claim 109, wherein the ceDNA comprises two mutant ITRs flanking both 5′ and 3′ends of the expression cassette.

128. The method of claim 95, wherein the TNA is a DNA-based minicircle or a MIDGE.

129. The method of claim 95, wherein the TNA is closed-ended DNA having at least one protelomerase target sequence.

130. The method of claim 95, wherein the TNA is a dumbbell shaped DNA.

131. The method of claim 95, wherein the TNA is a doggybone (dbDNA™) DNA.

132. The method of claim 95, wherein the TNA is a minigene.

133. The method of claim 95, wherein the TNA is a linear covalently closed DNA ministring.

134. The method of claim 95, wherein the TNA is a plasmid or bacmid.

135. The method of any one of claims 95-134, wherein the protein kinase inhibitor is a tyrosine kinase inhibitor (TKI), or a pharmaceutically acceptable salt thereof.

136. The method of any one of claims 95-134, wherein the TNA is formulated in a pharmaceutically acceptable excipient or carrier.

137. The method of claim 136, wherein the carrier comprises a lipid nanoparticle (LNP).

138. The method of claim 135, wherein the TNA is formulated in a pharmaceutically acceptable excipient.

139. The method of claim 135, wherein the TKI is a small molecule.

140. The method of claim 135, wherein the TKI is a biologic agent.

141. The method of claim 135, wherein the LNP comprises the protein kinase inhibitor.

142. The method of any one of claims 95-134, wherein the protein kinase inhibitor is a tyrosine kinase inhibitor (TKI).

143. The method of claim 142, wherein the TKI is selected from the group consisting of acalabrutinib, alectinib, baricitinib, afatinib, brigatinib, crizotinib, dacomitinib, dasatinib, lorlatinib, osimertinib, fostamatinib, saracatinib, AG-1478, cobimetinib, ceritinib, lapatinib, gefitinib, erlotinib, TAK-659, ruxolitinib, cerdulatinib, tofacitinib, vandetinib, and bosutinib.

144. The method of claim 142, wherein the TKI is selected from the group consisting of baricitinib, afatinib, brigatinib, dacomitinib, dasatinib, osimertinib, fostamatinib, saracatinib, cobimetinib, ceritinib, ruxolitinib, cerdulatinib, and tofacitinib.

145. The method of claim 142, wherein the TKI is selected from the group consisting of sunitinib, imatinib, sorafenib, dasatinib, entoplestinib, fostamatinib, TAK-659, ruxolitinib, baricitinib, and tofacitinib.

146. The method of claim 142, wherein the TKI is an STAT1 inhibitor.

147. The method of claim 142, wherein the TKI is an inhibitor of spleen tyrosine kinase (Syk).

148. The method of claim 142, wherein the Syk inhibitor is fostamatinib.

149. The method of claim 142, wherein the Syk inhibitor is cerdulatinib.

150. The method of claim 142, wherein the TKI is an inhibitor of epidermal growth factor receptor (EGFR, aka ErbB-1 or HER-1).

151. The method of claim 150, wherein the EGFR inhibitor is afatinib.

152. The method of claim 150, wherein the EGFR inhibitor is dacomitinib.

153. The method of claim 142, wherein the TKI is an anaplastic lymphoma kinase (ALK) inhibitor.

154. The method of claim 153, wherein the ALK inhibitor is brigatinib.

155. The method of claim 153, wherein the ALK inhibitor is alectinib.

156. The method of claim 153, wherein the ALK inhibitor is ceritinib.

157. The method of claim 153, wherein the ALK inhibitor is lorlatinib.

158. The method of claim 153, wherein the ALK inhibitor is crizotinib.

159. The method of claim 142, wherein the TKI is an antagonist of IFN production pathway.

160. The method of claim 142, wherein the TKI is an antagonist of IFN signaling pathway.

161. The method of claim 142, wherein the TKI is an inhibitor of tyrosine kinase 2 (Tyk2).

162. The method of claim 142, wherein the TKI is an inhibitor of Janus kinase 1 (Jak1).

163. The method of claim 142, wherein the TKI is an inhibitor of Janus kinase 2 (Jak2).

164. The method of claim 161, wherein the Tyk2 inhibitor is ruxolitinib.

165. The method of claim 162, wherein the Jak1 inhibitor is ruxolitinib.

166. The method of claim 163, wherein the Jak2 inhibitor is ruxolitinib.

167. The method of claim 162, wherein the Jak1 inhibitor is baricitinib.

168. The method of claim 163, wherein the Jak2 inhibitor is baricitinib.

169. The method of claim 161, wherein the Tyk2 inhibitor is tofacitinib.

170. The method of claim 142, wherein the TKI is an inhibitor of STAT1 or STAT2.

171. The method of claim 142, wherein the TKI is ABL/Src inhibitor.

172. The method of claim 95, wherein the subject is a human suffering from the genetic disorder.

173. The method of claim 172, wherein the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, phenylketonuria (PKU), Wilson's disease, congenital hepatic porphyria, inherited disorders of hepatic metabolism, Lesch Nyhan syndrome, sickle cell anemia, thalassemia's, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis (LCA), Stargardt macular dystrophy (ABCA4 deficiency), omithine transcarbamylase (OTC) deficiency, Usher syndrome, alpha-1 antitrypsin deficiency, progressive familial intrahepatic cholestasis (PFIC), and Cathepsin A deficiency.

174. The method of claim 173, wherein the genetic disorder is Parkinson's disease.

175. The method of claim 173, wherein the genetic disorder is Alzheimer's disease.

176. The method of claim 173, wherein the genetic disorder is thalassemia.

177. The method of claim 173, wherein the genetic disorder is Leber congenital amaurosis (LCA).

178. The method of claim 177, wherein the LCA is LCA10.

179. The method of claim 173, wherein the genetic disorder is Niemann-Pick disease.

180. The method of claim 173, wherein the genetic disorder is Stargardt macular dystrophy.

181. The method of claim 173, wherein the genetic disorder is Pompe disease (glycogen storage disease type II).

182. The method of claim 173, wherein the genetic disorder is hemophilia A (Factor VIII deficiency).

183. The method of claim 173, wherein the genetic disorder is hemophilia B (Factor IX deficiency).

184. The method of claim 173, wherein the genetic disorder is hunter syndrome (Mucopolysaccharidosis II).

185. The method of claim 173, wherein the genetic disorder is cystic fibrosis (CFTR).

186. The method of claim 173, wherein the genetic disorder is dystrophic epidermolysis bullosa (DEB).

187. The method of claim 173, wherein the genetic disorder is phenylketonuria (PKU).

188. The method of claim 173, wherein the genetic disorder is hyaluronidase deficiency.

189. The method of any one of claims 142 to 188, wherein the TKI is administered prior to the administration of the TNA.

190. The method of any one of claim 142 to 189, wherein the TKI is administered at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least about 2 days, at least about 3 days, at least about 4 days, at least about 5 days, at least about 6 days, at least about 1 week, at least about 2 weeks, at least about 3 weeks, or at least about 4 weeks prior to the administration of the TNA.

191. The method of any one of claims 142 to 188, wherein the TKI is administered simultaneously to the administration of the TNA.

192. The method of any one of claims 142 to 188 and 191, wherein the TKI and said TNA are in a liquid solution.

193. The method of claim 191, wherein the TKI and TNA are any of the pharmaceutical compositions according to claims 1 to 79.

194. The method of any one of claims 142 to 188, wherein the TKI is administered after the administration of the TNA.

195. The method of claim 194, wherein the TKI is administered 30 minutes after the administration of the TNA.

196. The method of claim 194, wherein the TKI is administered about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or 24 hours after the administration of the therapeutic nucleic acid.

197. The method of claim 194, wherein the TKI is administered about 1 day, about 2 days, about 3 days, about 4 days, about 6 days, or about 7 days after the administration of the TNA.

198. The method of claim 194, wherein the TKI is administered about 5 hours after the administration of the TNA.

199. The method of claim 194, wherein the TKI is administered about 12 hours after the administration of the TNA.

200. The method of claim 194, wherein the TKI is administered about 24 hours after the administration of TNA.

201. The method of claim 142, wherein the TKI is administered multiple times, before, concurrently with, and/or after the administration of the TNA.

202. The method of claim 142, wherein the TKI is administered multiple times, before and/or after the administration of the TNA, at least within about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or 24 hours.

203. The method of claim 142, wherein the TKI is administered multiple times, before, at the same time, and/or after the administration of the TNA.

204. The method of any of claims 95-203, wherein the protein kinase inhibitor and TNA are administered by oral, topical, intradermal, intrathecal, intravenous, subcutaneous, intramuscular, intratumoral, intra-articular, intraspinal, spinal, nasal, epidural, rectal, vaginal, transdermal, or transmucosal route.

205. The method of any one of claims 95-103, wherein the protein kinase inhibitor is administered at a dosage of about 0.5 mg/kg to about 700 mg/kg.

206. The method of claim 205, wherein the protein kinase inhibitor is administered at a dosage of about 100 mg/kg, about 150 mg/kg, about 200 mg/kg, about 250 mg/kg, about 300 mg/kg, about 350 mg/kg, or about 400 mg/kg.
